(19) United States
(12) Patent Application Publication
(10) Pub. No.: US 2009/0202635 A1
(43) Pub. Date: Aug. 13, 2009

(54) Delivery System, Application, and Method

(76) Inventor: Stephen Michael Scott, Tampa, FL (US)

Correspondence Address: STEPHEN M. SCOTT, 515 BOSPHORUS AVE, TAMPA, FL, 33606, US

(21) Appl. No.: 12/361,863

(22) Filed: Jan. 29, 2009

Related U.S. Application Data
(60) Provisional application No. 61/027,265, filed on Feb. 8, 2008.

(51) Int. Cl. A61K 9/12; A61K 9/20

(52) U.S. Cl. 424/468; 424/464; 424/45

(57) ABSTRACT

A sequenced biologically compatible delivery system, application, and method is provided, which increases therapeutic care of animals by conveniently and effectively supplying meticulous administration plans requiring sequential administration of varying biologically active agents.

### BACKGROUND OF THE PRESENT INVENTION

Administration plans for medications or other agents for those with special therapeutic requirements require an improved delivery system, application, and method for meticulous administration plans to assist users in proper therapeutic care, and to provide for monitoring of therapeutic care and track compliance with the meticulous administration plans. It is well documented that various groups of users do not follow administration plans, including the elderly, the infirm, children, and those who are otherwise alert, but are pre-occupied. The meticulous administration plan must be followed precisely because user errors can prevent efficacy and create health risks.

Many thin edible films may be used to deliver medicaments or other agents to a target bodily cavity or area as it has been known to administer biologically active agents in an edible film vehicle.

U.S. Pat. Nos. 7,132,113, 7,067,116, 7,025,983, 5,948,430, 5,700,478, 5,411,945, 5,047,244, 4,900,552, 4,876,092 describe films allowing the release of a biologically active agent.

Similarly, WO 99/17753, WO 98/26780, WO 98/20862, WO 98/26763, EP 0 200 508 B1, EP 0 381 194 B1, CA 1 263 312, DE 24 49 865.5, DE 36 30 603, EP 0 219 762, EP 0 452 466 B1 disclose films that release biologically active agents that are incorporated in the product.

These disclosures are deficient in several respects when considered in meticulous administration plans. First, they are limited in the quantity of actives that can be delivered by the relatively thin cross-section practical in production and consumption of thin films. Second, they provide only a single composition of any active agent. Finally, they provide no effective monitoring means for users or monitors to track compliance.

These preparations and methods also do not allow for sequential administration of different formulations of biologically active agents from a common biologically compatible carrier. They propose mono-product, homogenous delivery carriers that fail to provide a wide variety of biologically active agents from a common biologically compatible carrier. Further their innate geometries allow only for relatively low administration amounts of a single composition of active agent.

Therefor, these prior administrative forms known in the art fail to provide for sequential administration of different formulations of biologically active agents from a common biologically compatible carrier, and cannot provide adequate amounts of active agent in all cases.

Another problem is solid administration forms. Oral administration by swallowing solid forms is a prevalent method of administration. Oral administration is often preferred because it is convenient, comfortable, painless, and simple to accomplish for many users.

However, administration by swallowing suffers from several disadvantages. One disadvantage is that pediatric and geriatric users frequently have difficulty swallowing pills and other solid forms. Such users find swallowing liquid administrations inconvenient. Swallowing often requires fluids and increases gastric volume, increasing the incidence of nausea and vomiting.

In addition, there is a delay between the time of oral administration and the time that the therapeutic effect begins, and this delay is often substantial. Where rapid turnover of users is essential substantial delays are unacceptable.

Another problem with administration by swallowing is variation of the active agents in the bloodstream from user to user. This variation depends on the action of the stomach, the small and the large intestines, the effects of the secretions of these organs, the pH within these organs, and finally the rate of absorption into the bloodstream.

In addition, administrations are often for use with an ‘average’ user. The result may be underdosing or overdosing a particular user.

In addition, administration by swallowing routes active agents into the bloodstream via a first pass through the liver. More than sixty percent of most biologically active agents (and essentially one hundred percent of certain biologically active agents) are removed by the liver during this first pass.

The result is that swallowing is impractical for many biologically active agents as the above described factors can prevent or reduce efficacy.

As an alternative, injection is frequently used, allowing the biologically active agent to become rapidly distributed to various portions of the user's body before exposure to the liver. Injections are comparatively inconvenient, painful, and costly.

Alternatively, administration through the buccal mucosa of the cheek pouch or by sublingual administration is suggested, for example U.S. Pat. No. 4,671,953. Administration of therapeutic agents through the mucosal tissues of the mouth, pharynx, and esophagus offers advantages. The biologically active agent is not exposed to the gastric and intestinal digestive juices. In addition, the biologically active agents bypass the liver and are not immediately initially metabolized and inactivated.

Another problem is user errors. Improper administration of prescriptions occurs in about 30% to 50% of all users. (See Libow et al.) In addition, 50% of acute care users do not comply with their administration plan. (See Parkin et al.) Further, administration errors often produce illness. (See Seidl et al.) Finally, elderly users revealed 2.3 serious errors per user among 25%, while about 59% overall error in their administrations. (See Schwartz et al.)

Packages which are used to improve user compliance with administration plans have been previously disclosed.

U.S. Pat. No. 6,951,353 proposes an administration management system that includes a card or overlay with raised tabs that are pressed down after a pill is taken. U.S. Pat. No. 5,261,702 proposes a system that includes a chart. U.S. Pat. No. 4,815,767 is illustrative of a chart system for use in assisting a user in tracking an administrations plan.

Examples of blister packages for organizing administrations are set forth in U.S. Pat. Nos. 7,000,769, 6,375,956, 3,905,479.; 3,912,082; 3,924,747; 3,835,995; 3,912,081; 3,924,746; 3,809,220; 3,809,221; 3,811,564; 3,872,970; 3,899,080; 3,921,805; and 3,941,248.

Similarly, WO 01/07012, WO 98/22072, Great Britain Patent No. 2 228 922 disclose examples of blister packages.

U.S. Pat. Nos. 5,788,974, 5,695,063, 5,624,036, 5,358,118, 5,325,968, 5,310,060, 4,958,736, 4,889,236, 4,295,567 disclose other packages for organizing administrations.

These proposals include combining various biologically active agents, various dosage forms, and various administrations in various packaging. These approaches require the biologically active agents to be separately handled. They also require custom packaging.

The aforementioned holders, dispensers and packages merely repackage various compositions and forms into proposed packaging. They are deficient in several respects. First, they require additional handling as each administration must be loaded.

Second, they require additional packaging to organize the administrations. Finally the feedback for tracking compliance relies on the user.

Significantly, none presents a convenient, cost-friendly, simple and effective way of facilitating sequential administration of varying isolated formulations of biologically active agents from a common biologically compatible carrier. Thus, they fail to provide for sequential administration of different formulations of biologically active agents from a common biologically compatible carrier. Further, they fail to provide adequate feedback mechanisms in the event of user misuse or user error. In addition, they fail to provide a positive means to prevent user misuse or user error. Finally the thin film solutions do not provide adequate amounts of active agents. These shortcomings are particularly critical when present during a meticulous administration plan. They also add significant cost from added handling and packaging.

There are a broad range of factors which play a role in poor user compliance, including poor user memory, user ignorance, user apathy, user cognitive difficulties, and complexity of administration plan. These same causes challenge the aforementioned proposals in pursuit of improved meticulous administration plans.

Thus, there is demand for a system and method that is a simple, effective, cost-friendly, and convenient means for providing improved therapeutic care, and in particular for providing improved care when there are special therapeutic requirements.

The present invention discloses compositions for systems, and applications, and methods that are capable of providing precise sequential administration of varying biologically active agents from a common biologically compatible carrier with a simple, effective, cost-friendly, and convenient means for providing improved therapeutic care, and in particular for providing improved care when there are special therapeutic requirements, enabling improved compliance with a meticulous administration plan, consisting of varying administrations of biologically active agents over a period of time.

The inventor is not aware of any suggestion in the published art that compliance with a meticulous administration plan, consisting of varying administrations of biologically active agents over a period of time, can be achieved using a sequenced biologically compatible delivery system, application, and method that uses a common biologically compatible carrier to organize varying isolated formulations of biologically active agents.

Despite the existence of homogeneous films, mono-product, and multi-product blister packs in the prior art, there is need for new compositions, applications, and methods for making them.

Thus, there is a need for delivery a system for biologically active agents, and applications therefor, and methods therefor, that do not suffer from the foregoing disadvantages, enabling compliance with a meticulous administration plan, consisting of varying administrations of biologically active agents over a period of time.

All references, including any patents or patent applications cited in this specification are hereby incorporated by reference in their entireties. No conclusion is reached that any reference constitutes prior art. The discussion of the references states what their authors assert, and the applicant reserves the right to challenge the accuracy and pertinence of the cited documents. It will be clearly understood that, although a number of prior art publications are referred to herein, these references do not conclude that any of these documents form part of the common general knowledge whether in the art, or in any country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning, for example an inclusion of not only the listed components it directly references, but also other non-specified, or otherwise components or elements. This rationale will also be used whenever any of the terms ‘wherein’ or ‘comprised’ or ‘comprising,’ or combinations thereof, are used in relation to one or more steps in a composition, system, application, or method.

### BRIEF SUMMARY OF THE PRESENT INVENTION

It has been discovered that a simple, effective, cost-friendly, and convenient means for providing improved therapeutic care, and in particular for providing improved care when there are special therapeutic requirements, can be realized from the use of a sequenced biologically compatible delivery system, application, and method using a common biologically compatible carrier, enabling compliance with a meticulous administration plan, consisting of varying administrations of biologically active agents over a period of time. The system, application, and method provides improvements in therapeutic care while overcoming the deficiencies of currently available solutions in a simple, effective, convenient and cost-friendly manner.

The present invention relates to a sequenced biologically compatible delivery system, application, and method for sequentially administering biologically active agents to animals, where the biologically active agents are organized on a common biologically compatible carrier. Specifically, the present invention relates to a biologically compatible array consisting of a plurality of sites of varying isolated formulations of biologically active agents organized on a common biologically compatible carrier.

The present invention sequentially provides drugs, nutritionals, cosmeceuticals, hormones, antivirals, and diagnostic agents, without limitation, sequenced as a plurality of sites of varying isolated formulations of biologically active agents organized on a common biologically compatible carrier.

The system, application, and method of the present invention are designed for improved levels of user error, user monitoring, cost reduction, user convenience, production variation, and distribution in conjunction with a meticulous administration plan, consisting of varying administrations of biologically active agents over a period of time.

It is an object of the present invention to provide a sequenced biologically compatible delivery system comprising a plurality of sites of varying isolated formulations of biologically active agents organized on a common biologically compatible carrier.

It is another object of the present invention to provide a system wherein the biologically active agents are sequenced as a plurality of sites of varying isolated formulations of biologically active agents organized on a common biologically compatible carrier.

It is another object of the present invention to provide a system wherein the biologically active agents are independently isolated in close proximity unit to prevent interaction between the biologically active agents while facilitating sequential administration of the biologically active agents.

It is another object of the present invention to provide a system that delivers a wide variety of biologically active agents organized on a common biologically compatible carrier.

It is another object of the present invention to provide a system that delivers a prescription agent and a nonprescription agent in concurrent sequential administrations, organized on a common biologically compatible carrier.

It is another object of the present invention to provide a system comprising only one layer of reagent formulations emplaced on the biologically compatible carrier.

It is another object of the present invention to provide a system that delivers large quantities of biologically active agents per administration of a single site, using one or more layers of reagent formulations emplaced on the biologically compatible carrier.

It is another object of the present invention to provide a system comprising a multiplicity of layers of reagent formulations emplaced on the biologically compatible carrier.

It is another object of the present invention to provide a system comprising varying layers of reagent formulations emplaced on the biologically compatible carrier.

It is another object of the present invention to provide a system that delivers uneven or varying administrations of a biologically active agent, organized on a common biologically compatible carrier.

It is another object of the present invention to provide a system that sequentially delivers greater or lesser amounts by weight of the biologically active agents, organized on a common biologically compatible carrier.

It is another object of the present invention to provide a system that sequentially delivers administrations that vary by volume or shape, organized on a common biologically compatible carrier.

It is another object of the present invention to provide a system that delivers user specific types and quantities of biologically active agents, organized on a common biologically compatible carrier.

It is another object of the present invention to provide a system that does not require the user to swallow any form of solid or semi-solid object, or drink any liquid.

It is another object of the present invention to provide a system comprising biologically compatible control codes and biologically compatible user instructions for each formulation of biologically active agents on the biologically compatible carrier.

It is another object of the present invention to provide a system wherein the biologically compatible control codes determine which formulations of biologically active agents are emplaced on the biologically compatible carrier.

It is another object of the present invention to provide a system wherein the biologically compatible control codes determine where formulations of biologically active agents are emplaced on the biologically compatible carrier.

It is another object of the present invention to provide a system wherein the biologically compatible control codes can verify whether the user has administered the correct formulation of biologically active agents from the biologically compatible carrier.

It is another object of the present invention to provide a system wherein the biologically compatible control codes can verify whether the user has administered the formulation of biologically active agents from the biologically compatible carrier at the correct time.

It is another object of the present invention to provide a system comprising biologically active agents for a variety of bodily cavities, sequenced as a plurality of sites of varying isolated formulations of biologically active agents organized on a common biologically compatible carrier.

It is another object of the present invention to provide a system, wherein the sites administered to a user rapidly dissolve or disintegrate.

It is another object of the present invention to provide a system, wherein the sites administered to a user undergo controlled or sustained disintegration.

It is another object of the present invention to provide a system wherein unpalatable biologically active agents are taste masked to improve palatability.

It is another object of the present invention to provide a system wherein the chemical stability of the biologically active agents is increased.

It is another object of the present invention to provide a system wherein the absorption and/or bioavailability of biologically active agents is improved.

It is another object of the present invention to provide a system wherein each of the isolated formulations of biologically active agents has a seal that is independently accessible, removable or breakable.

It is another object of the present invention to provide a system whereas the sites are optionally produced by the processing of solid powders or liquids at room temperature, preventing the degradation of biologically active agents.

It is another object of the present invention to provide a system wherein the use of biologically active agents having relatively low melting points, or wherein the use of those biologically active agents which can experience decomposition below their melting points, is facilitated.

It is another object of the present invention to provide a system whereas the system is optionally produced so as to reduce evaporation of volatile components.

It is another object of the present invention to provide a system whereas the system is optionally produced so as to allow components which may be chemically inappropriate when in a heated solution or suspension.

It is another object of the present invention to provide a system whereas the system is optionally produced so as to allow biologically active agents, flavorings, and other components which are insoluble when placed in the same liquid environment to be processed as part of a compressible reagent formulations.

In accordance with these and other objects, the present invention provides a sequenced biologically compatible delivery system, application, and method for delivery of a wide variety of biologically active agents where the biologically active agents are sequenced as a plurality of sites of varying isolated formulations of biologically active agents organized on a common biologically compatible carrier.

The system, application, and method of the present invention are designed for improved levels of user error, user monitoring, cost reduction, user convenience, production variation, and distribution when delivering varying isolated formulations of biologically active agents, organized on a common biologically compatible carrier, over a period of time.

In one embodiment, the sequenced biologically compatible delivery system comprises a (I) biologically compatible array, a (II) tamper resistant pack, and (III) outer packaging, wherein the biologically compatible array further comprises (i) a biologically compatible carrier, (ii) biologically compatible control codes, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area, and (V) the methods for producing the system of the present invention.

In another embodiment, the sequenced biologically compatible delivery system includes a (I) biologically compatible array, a (II) tamper resistant pack, and (III) outer packaging, wherein the biologically compatible array further comprises (i) a biologically compatible carrier including one or more biologically active agents, (ii) biologically compatible control codes, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents irito, onto, or near the target bodily cavity or area , and (V) the methods for producing the system of the present invention.

In another embodiment, the sequenced biologically compatible delivery system comprises a (I) biologically compatible array, a (II) tamper resistant pack, and (III) outer packaging, wherein the biologically compatible array further comprises (i) a biologically compatible carrier, (ii) biologically compatible control codes that determine which biologically active agents are emplaced on the biologically compatible carrier, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area , and (V) the methods for producing the system of the present invention.

In another embodiment, the sequenced biologically compatible delivery system comprises a (I) biologically compatible array, a (II) tamper resistant pack, and (III) outer packaging, wherein the biologically compatible array further comprises (i) a biologically compatible carrier, (ii) biologically compatible control codes that determine where the biologically active agents are emplaced on the biologically compatible carrier, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area , and (V) the methods for producing the system of the present invention.

In another embodiment, the sequenced biologically compatible delivery system comprises a (I) biologically compatible array, a (II) tamper resistant pack, and (III) outer packaging, wherein the biologically compatible array further comprises (i) a biologically compatible carrier, (ii) biologically compatible control codes that determine whether the biologically active agents are administered in the correct sequence, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area , and (V) the methods for producing the system of the present invention.

In another embodiment, the sequenced biologically compatible delivery system comprises a (I) biologically compatible array, a (II) tamper resistant pack, and (III) outer packaging, wherein the biologically compatible array further comprises (i) a biologically compatible carrier, (ii) biologically compatible control codes that determine whether the biologically active agents are administered at the correct time, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area , and (V) the methods for producing the system of the present invention.

In other aspects, the present invention also provides for a delivery system for any of the biologically active agents, applications to administer any of the biologically active agents, and methods of producing the delivery systems.

These and other features, advantages and objects of the present invention will become more fully apparent, understood and appreciated by those skilled in the art by reference from the following specification and appended claiims, or may be learned by the practice of the present invention as set forth hereinafter, wherein a novel sequenced biologically compatible delivery system, application, and method is disclosed.

### BRIEF DESCRIPTION OF THE SEVERAL VIEWS OF THE DRAWINGS

The accompanying drawings illustrate various embodiments of the system, application, and method of the present invention and are a part of the specification. Throughout the drawings, identical reference numbers designate similar, but not necessarily identical, elements. Further aspects of the present invention will become apparent from the illustrated embodiments, which are merely examples of the present invention and do not limit the scope thereof, and with reference to the accompanying drawings in which:

FIG. 1 is a plan view illustrating one embodiment of the present invention, showing a daily system for the administration of biologically active agents from a biologically compatible array, showing alignment of a single site and three additional sites, all varying, for sequential administration, where one row of sites is used.

FIG. 2A is a plan view illustrating another embodiment of the present invention, showing a once per day system for the administration of biologically active agents from a biologically compatible array, showing alignment of twenty-eight varying sites for sequential administration, where four rows of sites are used.

FIG. 2B is a cross-sectional side-view illustrating a portion of the biologically compatible array shown in FIG. 2A.

FIG. 2C is a plan view illustrating a portion of the biologically compatible carrier shown in FIG. 2A.

FIG. 2D is a plan view illustrating a portion of the biologically compatible user instructions shown in FIG. 2A.

FIG. 2E is a plan view illustrating a portion of the biologically compatible control codes shown in FIG. 2A.

FIG. 2F is a plan view illustrating four of the sites shown in FIG. 2A.

FIG. 2G is a plan view illustrating one site separated from the portion of the biologically compatible array shown in FIG. 2F.

FIG. 3C is a cross-sectional side-view illustrating a portion of the biologically compatible carrier shown in FIG. 2C, looking through view IIC.

FIG. 3D is a cross-sectional side-view illustrating a portion of the biologically compatible user instructions shown in FIG. 2D, looking through view IID.

FIG. 3E is a cross-sectional side-view illustrating a portion of the biologically compatible control codes shown in FIG. 2E, looking through view IIE.

FIG. 3F is a cross-sectional side-view illustrating a portion of the sites shown in shown in FIG. 2F, looking through view IIF.

FIG. 3G is a cross-sectional side-view illustrating one site separated from a portion of the biologically compatible array shown in FIG. 2G.

FIG. 4A is a plan view illustrating another embodiment of the present invention, showing a daily system for the administration of biologically active agents from a biologically compatible array, showing alignment of a single site and three additional sites, all varying, for sequential administration, where seven rows of sites is used.

FIG. 4B is a plan view illustrating the embodiment of FIG. 4A, showing two sites in the biologically compatible array shown in FIG. 4A.

FIG. 4C is a plan view illustrating the embodiment of FIG. 4B, showing two sites after separation from the biologically compatible array shown in FIG. 4B.

FIG. 5A is a plan view illustrating another embodiment of the present invention, showing alignment of a site and additional varying sites for sequential administration, suitably compatible user instructions and pull-tab safety seals, showing the system in a tamper resistant pack.

FIG. 5B is a plan view illustrating another embodiment of the present invention, showing alignment of a site and additional varying sites for sequential administration, showing suitably compatible user instructions and pull-tab safety seals, showing the system in a tamper resistant pack with a safety seal being partially removed, and showing one site after separation from the biologically compatible array shown in FIG. 5A.

FIG. 6A is a plan view illustrating another embodiment of the present invention, showing alignment of a site and additional varying sites for sequential administration, suitably compatible user instructions and blister safety seals, showing the system in a tamper resistant pack.

FIG. 6B is a plan view illustrating another embodiment of the present invention, showing alignment of a site and additional varying sites for sequential administration, showing suitably compatible user instructions and blister safety seals, showing the system in a tamper resistant pack with a safety seal being partially removed, and showing one site after separation from the biologically compatible array shown in FIG. 6A.

FIG. 7A is a perspective view illustrating another embodiment of the present invention, showing layers within the interior of a site.

FIG. 7B is a perspective view illustrating another embodiment of the present invention, showing segments within the interior of a site.

FIG. 8A is a perspective view illustrating another embodiment of the present invention, showing the top plate suitable for molding the embodiment of FIG. 1.

FIG. 8B is a perspective view illustrating another embodiment of the present invention, showing the cavity mold plate for molding the embodiment of FIG. 1.

FIG. 8C is a perspective view illustrating another embodiment of the present invention, showing the die suitable for molding the embodiment of FIG. 1.

FIG. 9A is a perspective view illustrating another embodiment of the present invention, showing the assembled mold of FIG. 8A, FIG. 8B, and FIG. 8C, with the embodiment of FIG. 1 within the mold.

FIG. 9B is a perspective view illustrating the partially assembled mold of FIG. 8A, FIG. 8B, and FIG. 8C, with the embodiment of FIG. 1 molded, showing the top plate of FIG. 8A after molding and removed from the mold set.

FIG. 9C is a top view illustrating the embodiment of FIG. 1 after removal from the mold cavity.

FIG. 9D is a cross-sectional side-view illustrating a portion of the biologically compatible array shown in FIG. 9C.

FIG. 10 is a perspective view illustrating another embodiment of the present invention, showing an outer packaging for storing a plurality of biologically compatible delivery systems.

### DETAILED DESCRIPTION OF THE PRESENT INVENTION

As used herein:

‘Administration’ refers to each individual release of biologically active agent(s) into, onto, or near body tissue or target bodily cavities, without limitation.

‘Administrations’ refers to two or more administrations, which may be concurrent, spread over any period of time, or planned for over a period of time, and various combinations thereof, without limitation.

‘Agent’ refers to any biologically active agent, as defined, without limitation.

‘Animal’ refers to a human, mammal or any other animal, without limitation.

‘Biologically compatible’ refers to being safe for animal or human contact, consumption, exposure, or insertion, as defined, without limitation.

‘Biologically compatible array’ refers to the portion of the present composition of the system and intermediate step in the present method comprising the configuration of sites further comprising the biologically compatible carriers, biologically compatible control codes, biologically compatible user instructions, biologically active agents, and biologically compatible reagent formulations, without limitation.

‘Biologically compatible carrier’ refers to the portion of the present composition of the system and intermediate step in the present method comprising the present delivery system further comprising the supporting and organizing structure for the sites, without limitation.

‘Biologically compatible control code’, or ‘control code’, or ‘biologically compatible indicia’, or ‘indicia’ refer to the portion of the present composition of the system and intermediate step in the present method comprising machine feedback or instruction, or any combination of the above that is biologically compatible or suitably compatible, as defined, without limitation.

‘Biologically compatible user instruction’, or ‘user instruction’ or ‘biologically compatible indicia’, or ‘indicia’ refer to the portion of the present composition of the system and intermediate step in the present method comprising human feedback or instruction, or any combination of the above that is biologically compatible or suitably compatible, as defined, without limitation.

‘Biologically active agent’ refers to the portion of the present composition of the system and intermediate step in the present method comprising any agent or agents including a drug, active ingredient, metabolite, medicament, hormone, steroid, vitamin, fatty acid, amino acid, sugar, carbohydrate, polypeptide or mineral, any agent used for treatment, prevention, diagnosis, cure or mitigation of disease or illness, any agent which affects anatomical structure or physiological function, or any agent which alters the impact of external influences on an animal, or metabolite thereof, and as used herein, encompasses the terms ‘active agent’, ‘biologically active agent’, ‘agent’, ‘active agent’, ‘active ingredient’, ‘drug’, ‘medicine’, ‘medicament’, ‘nutritional’, ‘cosmeceutical’, ‘hormone’, ‘antiviral’, ‘diagnostic agent’ other like material, derivatives thereof, and various combinations thereof, without limitation.

‘Biologically compatible reagent formulations’ , or ‘reagent formulations’ refer to the portion of the present composition of the system and intermediate step in the present method comprising biologically compatible materials supporting effective delivery of and admixed with the biologically active agents, as defined, without limitation.

‘Essential fatty acids’ refers to any fatty acid that may be utilized by the body, and includes chemical chains of carbon, hydrogen, and oxygen atoms that are part of a fat (lipid), are a major component of triglycerides, which may be classified as either saturated, polyunsaturated, or monounsaturated, and may be found in nature or produced synthetically, other like material, derivatives thereof, and various combinations thereof, without limitation.

‘Incompatible agents’ refers to biologically active agents that may not be formulated together in a single administration or stored together in direct contact because the biologically active agents will be adversely effected and also biologically active agents that cannot be formulated together in a single administration because the resulting total of the administration amounts of the biologically active agents would result in a single administration which is too large for administration. The term also refers to biologically active agents that may be stored in direct contact, however one of the biologically active agents is preferably formulated in an administration which is either not preferred or incompatible with the other biologically active agents. Incompatible agents also refers to two or more biologically active agents wherein at least one biologically active agent is a prescription agent and at least one biologically active agent is a non-prescription agent.

‘Incompatibility’ refers to the state that exists between incompatible agents, as defined above.

‘Isolated’ or ‘isolation’ refers to the state that exists between sites in the biologically compatible array wherein the biologically active agents comprising one or more sites are physically, geometrically, or chemically organized to prevent undesirable interactions between incompatible agents, as defined above.

‘Meticulous administration plan’ refers to systematic administration of multiple administrations of a biologically active agent or biologically active agents at designated times during a period of time including, without limitation, multiple administrations and uneven administrations which are potentially confusing or impractical to a user, or to administer to a user.

‘Monitors’ or ‘Monitorees’ refer to any entity that makes use of the methods, systems, and applications described herein to monitor, and includes associations, companies, organizations, practitioners, patients, and various combinations thereof, without limitation, provided a user and monitoree as defined, may not be the same entity.

‘Nutritional status’ refers to the presence or absence of any nutrient deficiency, or the extent to which physiological nutrient demands are being satisfied such that deficiency is avoided.

‘Sequenced biologically compatible delivery system’ refers to a system, application, and method, comprising a biologically compatible array, tamper resistant pack, and outer packaging, the biologically compatible array further comprising a biologically compatible carrier with two or more sites organized on a common biologically compatible carrier, inside each of which site is contained one or more biologically active agents, which are isolated on the biologically compatible carrier and within the sites by reagent formulations, enabling compliance with a meticulous administration plan, consisting of varying administrations of biologically active agents over a period of time. The application of the present invention is sequentially administering biologically active agents to animals, where the biologically active agents are organized on a common biologically compatible carrier. The method of producing the present invention is such that the sites are independently accessible, separable, or breakable, and contain varying biologically active agents.

‘Shelf stability’ or ‘storage stability’ refers to the ability to resist degradation or alteration in chemical, physical or biological properties while awaiting use during a period of at least six months.

‘Site’ refers to any shape of isolated mixture of biologically compatible agent(s) and encompasses the terms ‘portion’, ‘spot’, ‘dot’, and the like, without limitation.

‘Sites’ refers to any plurality of sequenced sites of varying isolated formulations of biologically active agents organized on a common biologically compatible carrier, comprising any formulation of biologically active agents admixed with any combination of reagent formulations, as defined, without limitation.

‘Sequenced sites’ refers to any plurality of sequenced sites of varying isolated formulations of biologically active agents organized on a common biologically compatible carrier according to a meticulous administration plan.

‘Specific therapeutic requirements’ refers to the unique requirements for certain levels of certain biologically active agents by one or more classes of persons, such as children, women, seniors, or persons with specific conditions, such as the chronically ill, lactating women, pregnant women, etc., as distinguished from other classes.

‘Suitably compatible’ refers to any state of compatibility, including biologically compatible, or otherwise compatible with the various systems, applications, and methods of the present invention, without limitation.

‘The system of the present invention’, ‘the system’ or ‘system’ refer to the composition of the disclosed sequenced biologically compatible delivery system, without limitation.

‘The application of the present invention’, ‘the application’ or ‘application’ refer to the use of the system by a user to perform administrations, without limitation.

‘The method for producing the system of the present invention’, ‘the method’ or ‘method’ refer to production processes used to make the system, without limitation.

‘Time’ refers to any time of day, any day of week, month or year, or any month of year, and combinations thereof, without limitation.

‘Uneven’ or ‘unevenly’ refers to administration of biologically active agent(s) wherein at least one administration following the initial administration contains different amount(s) or type(s) of biologically active agent(s) than the previous administration.

‘User’ refers to any entity that makes use of the system, and applications, and methods described herein, and includes associations, companies, organizations, practitioners, patients, and various combinations thereof, without limitation.

‘Varying’ refers to the difference in biologically active agent(s) as defined in ‘Uneven’ or ‘unevenly’, herein.

The present invention provides a sequenced biologically compatible delivery system, application, and method that deliver biologically active agents to into, onto, or near the target bodily cavity or area of a user. The present invention comprises a (I) biologically compatible array, a (II) tamper resistant pack, and (III) outer packaging. The biologically compatible array further comprises (i) a biologically compatible carrier, (ii) biologically compatible control codes, (iii) biologically compatible user instructions, (iv) a plurality of sequenced sites of varying isolated formulations of biologically active agents, and (v) reagent formulations supporting effective delivery of and admixed with the biologically active agents, along with (IV) applications to perform administrations of the present invention, characterized by consisting essentially of: (a) the user administering varying biologically active agents(s) from the biologically compatible array to the target bodily cavity or area, (b) allowing the biologically active agents(s) to dissolve within, on, or near the target bodily cavity or area; and (c) releasing the biologically active agents into, onto, or near the target bodily cavity or area, and (V) the methods for producing the system of the present invention.

The present invention is a sequenced biologically compatible delivery system which provides improved therapeutic.and/or nutritional care to an animal by enabling compliance with a meticulous administration plan and facilitating administration of incompatible agents, and in particular, sequential administration of incompatible agents.

The present system comprises various types of biologically compatible arrays. The present biologically compatible arrays are characterized by a plurality of varying isolated formulations of biologically active agents referred to herein as ‘sites’. Each site comprises an administration and isolates that administration from the other administrations.

Thus, the biologically active agents within each administration are isolated from the biologically active agents of the other administrations, although they are in close proximity with other administrations on the biologically compatible array. This organization, particularly when including biologically compatible control code and user instructions corresponding to the sites, eases effective sequential administration of incompatible agents as required by a meticulous administration plan, providing improved therapeutic care.

(I) Biologically Compatible Array

Preferably, the system of the present invention further comprises a biologically compatible array.

The biologically compatible array of the present invention may be organized in a wide variety of configurations, without limitation. Generally, the biologically compatible array comprises at least one row of a plurality of isolated formulations of biologically active agents, each isolated mixture defining one site. Any suitable biologically compatible array organization may be used that provides for sequential administration of components, which may not be in direct contact, for extended storage. Thus, each site is designed to hold one administration having either multiple compatible biologically active agents or one incompatible biologically active agent with one or more compatible biologically active agents.

The sites are organized such that the first administration and additional administrations may be easily separated together and administered together. The sequential administration may be accomplished in a wide variety of ways.

The sequential administration may be accomplished by close proximity of the first and additional administrations on the biologically compatible array, by user instructions which indicate both are to be sequentially administered, by a safety device which makes the administrations sequentially available, by techniques for sequentially separating or separating one or more administrations from the biologically compatible array, or any combination of the above, without limitation.

Each site is visibly marked by at least one line defining the adjacent site. This line can be perforated. Two or more sites may contain agents which are incompatible, provided at least one site on the biologically compatible array contains incompatible agents in separate sites.

The biologically compatible arrays of the present invention can be configured in a sheet formation, rolled formation, multidimensional formation, or any other formation, or combination of formations, without limitation.

In one embodiment, the biologically compatible array is configured in a planar formation such that the sites form a series of rows and columns.

In an alternative embodiment, the biologically compatible array is configured in a rolled formation such that as a strip of sites is pulled, the administration to be taken next is revealed from the container or outer packaging.

In other alternative embodiments, the biologically compatible arrays of the present invention can be configured in a sheet formation, rolled formation, multidimensional formation, or any other formation, or combination of formations, without limitation.

Thus, the system of the present invention is not strictly limited to geometries that are planar. Any efficacious geometry is suitable, provided the biologically compatible array may be produced using biologically compatible components. Non-limiting exemplary geometries include bottles, canisters, packets, tubes, vials, and the like.

Unless specified otherwise, the term ‘%’ as used herein with reference to the final product (i.e., the system, as opposed to the formulation used to create it), denotes the percentage of the total dry weight contributed by the subject component. This theoretical value can differ from the experimental value, because in practice, the present composition and system typically retains some of the volatiles used in preparation.

The biologically compatible array further comprises (i) a biologically compatible carrier, (ii) biologically compatible control codes, (iii) biologically compatible user instructions, (iv) a plurality of sequenced sites comprising varying isolated formulations of biologically active agents, and (v) reagent formulations supporting effective delivery of and admixed with the biologically active agents.

(i) Biologically Compatible Carrier

Preferably, the system of the present invention further comprises a biologically compatible carrier.

Preferred biologically compatible carriers, according to the present invention comprise various types of biologically compatible carriers, without limitation.

Any biologically compatible carrier can be used that is biologically compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

Preferred biologically compatible carriers, according to the present invention optionally comprise at least one component from each of the groups further comprising binding agents, coloring agents, cooling agents, emulsifying agents, fillers, flavoring agents, glycols, humectants, plasticizers, preservatives, release agents, saliva stimulating agents, stabilizers, starches, surfactants, sweeteners, water soluble polymers, water, other like material, derivatives thereof, and various combinations thereof, without limitation.

Preferred water soluble polymers used in the biologically compatible carriers according to the present invention optionally comprise one or more selected from the groups consisting of acrylic acid polymers, alginates, biosynthetic gums, biosynthetic process starches, cellulose ethers, cellulosic compounds, cellulosic materials, edible polymers, gelatins, glycols, hydrocolloid flours, land plant extracts, modified starches, natural fiber extracts, natural gums, natural plant exudates, natural seaweeds, natural seed gums, polyacrylates, polyethylene oxides, seaweed extracts, vinyl polymers, other like material, derivatives thereof, and various combinations thereof, without limitation.

The water soluble polymers used in the biologically compatible carriers according to the present invention comprise one or more selected from the group consisting of acacia gum, agar gum, algin gum, amylose, arabic gum, calcium alginate, carboxymethylcellulose, carboxyvinyl polymer, carrageenan, casein, cellulose acetate butyrate, cellulose acetate phthalate, chitin, chitosan, collagen, dextran, dextrin, elsinan, ethylcellulose, gelatin, ghatti gum, gluten, guar gum, high amylose starch, hydroxyethylcellulose, hydroxyethylmethylcellulose, hydroxypropylated high amylose starch, hydroxypropylcellulose, hydroxypropylmethylcellulose, levan, locust gum, methylcellulose, methylmethacrylate copolymer, natural fiber extracts, natural plant exudates, natural seaweeds, natural seed gum, pectin, polyacrylic acid, polyethylene glycol, polyethylene, polyvinyl alcohol, polyvinylpyrrolidone, pullulan, sodium alginate, sodium carboxymethylcellulose, soy protein isolate, tara gum, tragacanth gum, whey protein isolate, xanthan gum, zein, other like material, derivatives thereof, and various combinations thereof, without limitation.

Suitable water soluble polymers include, but are not limited to, Carbopol®, which may be obtained from B.F. Goodrich, Sentry Polyox®, available from Union Carbide, Gantrez®, which may be obtained from GAF., Methocel® and Klucel®, both may be obtained from the Dow Chemical Company, hydroxyethyl cellulose, carboxymethyl cellulose, sodium carboxymethyl cellulose, methyl cellulose, ethyl cellulose, cellulose acetate phthalate, cellulose acetate butyrate, and the like.

The biologically compatible carrier may also comprise two or more suitable polymers in combination, for example, a carbomer combined with a polyethylene oxide, or a cellulosic compound combined with a gelatin, in an approximately 1:5 to 5:1 ratio.

A preferred water soluble polymer is hydroxypropylmethylcellulose, in amounts ranging from about 0.01 to about 99%, preferably about 30 to about 80%, more preferably from about 45 to about 70% of the biologically compatible carrier and even more preferably from about 60 to about 65% of the biologically compatible carrier.

The binding agents used in the biologically compatible carriers according to the present invention optionally comprise one or more selected from the group consisting of dry binders, film binders, and chemical binders, without limitation. Examples of dry binders are dry starch, dry sugars, starch, gelatin and sugars such as sucrose, dextrose, molasses, and lactose. Examples of film binders are celluloses, hydroxypropylmethylcellulose, ethylcellulose, or other suitable cellulose derivatives. Examples of chemical binders are sugar syrups, corn syrup, gums, water soluble polysaccharides such as acacia, tragacanth, guar and alginates, gelatin, gelatin hydrolysate, agar, sucrose, dextrose, and non-cellulosic binders, such as vinyl pyrrolidone copolymers, PVP, PEG, starch paste, pregelatinized starch, sorbitol, and glucose. Other exemplary binders include povidone, acrylic and methacrylic acid co-polymers, pharmaceutical glaze, milk derivatives, whey, conventional binders, and those for compressed administrations which enhance adhesion and include starch, gelatin and sugars such as sucrose, dextrose, molasses, and lactose.

Suitable binding agents include, but are not limited to, starch, gelatin, maltodextrin, sucralose, and sugars such as sucrose, dextrose, molasses, lactose, and the like.

Preferred binding agents are maltodextrin, sucralose, and dextrose, in amounts ranging from about 0.01 to about 99%, preferably about 0.1 to about 70%, more preferably from about 1 to about 50% of the biologically compatible carrier and even more preferably from about 1 to about 25% of the biologically compatible carrier.

The coloring agents used in the biologically compatible carriers according to the present invention optionally comprise one or more selected from the group consisting of pigments such as titanium dioxide, natural food colors and dyes suitable for food, drug and cosmetic applications, including FD&C Blue No. 2, and Green No. 3, other like. material, derivatives thereof, and various combinations thereof, without limitation. A description of FD&C dyes may be found in the Kirk-Othmer Encyclopedia of Chemical Technology. (See Kirk-Othmer)

Suitable coloring agents include, but are not limited to, titanium dioxide, FD&C Blue No. 2, Green No. 3, Blue No. 1, Red No. 40, Yellow No. 6, and the like.

Preferred coloring agents are Blue No. 1, Red No. 40, and Yellow No. 6, in amounts ranging from about 0.001 to about 5%, preferably about .0.005 to about 4%, more preferably from about 0.01 to about 2% of the biologically compatible carrier and even more preferably from about 0.1 to about 1% of the biologically compatible carrier.

The cooling agents used in the biologically compatible carriers according to the present invention optionally comprise one or more selected from the group consisting of monomenthyl succinate, WS3, WS23, Ultracool II, other like material, derivatives thereof, and various combinations thereof, without limitation.

Suitable cooling agents include, but are not limited to, monomenthyl succinate, Ultracool 11, and the like.

A preferred cooling agent is monomenthyl succinate, in amounts ranging from about 0.001 to about 5%, preferably about 0.0.005 to about 4%, more preferably from about 0.01 to about 2% of the biologically compatible carrier and even more preferably from about 0.1 to about 1% of the biologically compatible carrier.

The emulsifying agents used in the biologically compatible carriers according to the present invention optionally comprise one or more selected from the group consisting of triethanolamine stearate, quaternary ammonium compounds, acacia, gelatin, lecithin, bentonite, veegum, other like material, derivatives thereof, and various combinations thereof, without limitation.

Suitable emulsifying agents include, but are not limited to, triethanolamine stearate, quaternary ammonium compounds, acacia, gelatin, lecithin, and the like.

A preferred emulsifying agent is gelatin, in amounts ranging from about 0.01 to about 99%, preferably about 1 to about 70%, more preferably from about 2 to about 50% of the biologically compatible carrier and even more preferably from about 5 to about 25% of the biologically compatible carrier.

The fillers used in the biologically compatible carriers according to the present invention optionally comprise one or more selected from the group consisting of aluminum silicate, calcium carbonate, calcium phosphate, calcium sulfate, clay, di-calcium phosphate, gelatin, ground limestone, gum, magnesium carbonate, magnesium silicate, mono-calcium phosphate talc, titanium dioxide, tri-calcium phosphate, other like material, derivatives thereof, and various combinations thereof, without limitation.

Suitable fillers include, but are not limited to, gelatin, gum, calcium carbonate, talc, titanium dioxide, and the like.

A preferred filler is calcium carbonate, in amounts ranging from about 0.01 to about 99%, preferably about 1 to about 70%, more preferably from about 2 to about 50% of the biologically compatible carrier and even more preferably from about 5 to about 25% of the biologically compatible carrier.

The flavoring agents used in the biologically compatible carriers according to the present invention optionally comprise one or more selected from the group consisting of natural and artificial flavors, other like material, derivatives thereof, and various combinations thereof, without limitation. Sweeteners and flavors need not be added to biologically compatible carriers intended for non-oral administration.

Preferred flavorings include anise, apricot, banana, blackberry, blueberry, butter rum, butterscotch, caramel, cherry, chocolate, citrus oil, citrus, clove oil, clove oils, coconut, coffee, cranberry, ethyl vanillin, eucalyptol, fruit essence, fruit punch, grape, grapefruit, honey, lemon, licorice, mango, marshmallow, melon, menthol, methyl salicylate, mint, mint oil, mocha, orange, peach, peppermint oil, raspberry, spearmint, spearmint oil, strawberry, thymol, artificial vanilla, vanilla cream, vanilla, watermelon, wintergreen oil, zinc gluconate, other like material, derivatives thereof, and various combinations thereof, without limitation.

Suitable flavoring agents include, but are not limited to, any of the aforementioned flavoring agents, and the like.

Preferred flavoring agents are any of the aforementioned flavoring agents, in amounts ranging from about 0.01 to about 40%, preferably about 0.5 to about 30%, more preferably from about 2 to about 25% of the biologically compatible carrier and even more preferably from about 5 to about 10% of the biologically compatible carrier.

Suitable glycols include, but are not limited to, ethylene glycol, diethylene glycol, propylene glycol, and the like.

A preferred glycol is propylene glycol, in amounts ranging from about 0.1 to about 25%, preferably about 0.5 to about 20%, more preferably from about 1 to about 15% of the biologically compatible carrier and even more preferably from about 2 to about 10% of the biologically compatible carrier.

Suitable humectants include, but are not limited to, glycerol, propanediol, and the like.

A preferred humectant is glycerol, in amounts ranging from about 0.01 to about 50%, preferably about 1 to about 40%, more preferably from about 2 to about 30% of the biologically compatible carrier and even more preferably from about 2 to about 10% of the biologically compatible carrier.

The plasticizers used in the biologically compatible carriers according to the present invention optionally comprise one or more selected from the group consisting of corn syrup, glycerin, hydrogenated starch hydrolysate, maltitol, polyethylene glycol, polyol, propylene glycol, sorbitol, other like material, derivatives thereof, and various combinations thereof, without limitation.

Suitable plasticizers include, but are not limited to, corn syrup, glycerin, maltitol, sorbitol, and the like.

Preferred plasticizers are maltitol and propylene glycol, in amounts ranging from about 0.01 to about 20%, preferably about 0.02 to about 15%, more preferably from about 0.03 to about 12% of the biologically compatible carrier and even more preferably from about 0.5 to about 10% of the biologically compatible carrier.

The antimicrobial agents, or preservatives used in the biologically compatible carriers according to the present invention optionally comprise one or more selected from the group consisting of ascorbic acid, boric acid, benzoic acid, and salts thereof, benzalkonium chloride, benzyl alcohol, chlorocresol, methyl hydroxybenzoate, parabens, phenols, potassium sorbate, propyl hydroxybenzoate, salts of edentate, sodium benzoate, sorbic acid, quaternary ammonium compounds other like material, derivatives thereof, and various combinations thereof, without limitation.

Methods for evaluating the efficacy of preservatives are known to those skilled in the art.

Preferred preservatives are sodium benzoate and potassium sorbate in amounts from about 0.001% to about 5%, preferably from about 0.01% to about 1%.

Suitable preservatives include, but are not limited to, sodium benzoate, potassium sorbate, benzalkonium chloride, chlorocresol, and the like.

Preferred preservatives include sodium benzoate and potassium sorbate, in amounts ranging from about 0.0001 to about 5%, preferably about 0.005 to about 4%, more preferably from about 0.002 to about 3% of the biologically compatible carrier and even more preferably from about 0.01 to about 1% of the biologically compatible carrier.

Suitable release agents include, but are not limited to, magnesium stearate, glycerol monolaurate, compritol 888 (glyceryl behenate), calcium stearate, and the like.

A preferred release agent is glycerol monolaurate, in amounts ranging from about 0.01 to about 25%, preferably about 0.02 to about 20%, more preferably from about 0.04 to about 10% of the biologically compatible carrier and even more preferably from about 0.5 to about 5% of the biologically compatible carrier.

The saliva stimulating agents used in the biologically compatible carriers according to the present invention optionally comprise one or more selected from the group consisting of citric, lactic, malic, succinic, ascorbic, adipic, fumaric, tartaric acids, other like material, derivatives thereof, and various combinations thereof, without limitation.

Suitable saliva stimulating agents include, but are not limited to, citric, lactic, malic, ascorbic, adipic, tartaric acids, and the like.

Preferred saliva stimulating agents are citric, malic, and ascorbic acids, in amounts ranging from about 0.01 to about 30%, preferably about 0.5 to about 20%, more preferably from about 1 to about 10% of the biologically compatible carrier and even more preferably from about 2.5 to about 5% of the biologically compatible carrier.

The stabilizers used in the biologically compatible carriers according to the present invention optionally comprise one or more selected from the group consisting of acacia gum, agar gum, algin gum, arabic gum, carrageenan, ghatti gum, guar gum, tara gum, tragacanth gum, locust gum, xanthan gum, other like material, derivatives thereof, and various combinations thereof, without limitation.

Suitable stabilizers include, but are not limited to, agar gum, carrageenan, guar gum, locust gum, xanthan gum, and the like.

Preferred stabilizers are carrageenan, guar gum, locust gum, and xanthan gum, in amounts ranging from about 0.01 to about 20%, preferably about 0.02 to about 15%, more preferably from about 0.05 to about 10% of the biologically compatible carrier and even more preferably from about 0.1 to about 5% of the biologically compatible carrier.

The starches used in the biologically compatible carriers according to the present invention optionally comprise one or more selected from the group consisting of acid and enzyme hydrolyzed corn and potato starches, any of several water-soluble polymers derived from a starch (e.g., corn starch, potato starch, tapioca starch) such as by acetylation, halogenation, hydrolysis (e.g., such as which an acid), or enzymatic action, any type of water-soluble modified starch, including but not limited to oxidized, ethoxyolated, cationic, lypophilic and pearl starch, maltodextrins including Maltrin® M100, Maltrin® M180, Maltrin® QD M550, and Maltrin® QD M600, all of which may be obtained from Grain Processing Corporation, and Pure-Cote® B792 modified corn starch, also available from Grain Processing Corporation, other like material, derivatives thereof, and various combinations thereof, without limitation.

Suitable starches include, but are not limited to, maltodextrins including Maltrin® M100, Maltrin® M180, Maltrin® QD M550, Maltrin® QD M600, and the modified corn starch Pure-Cote® B792, and the like.

Preferred starches are maltodextrins, in amounts ranging from about 0.01 to about 99%, preferably about 1 to about 70%, more preferably from about 2 to about 50% of the biologically compatible carrier and even more preferably from about 5 to about 25% of the biologically compatible carrier.

The surfactants used in the biologically compatible carriers according to the present invention optionally comprise one or more selected from the group consisting of Atmos 300, Polysorbate 80, pluronic acid, sodium lauryl sulfate, mono and diglycerides of fatty acids, polyoxyethylene sorbitol esters, polyoxyethylene sorbitan fatty acid esters, .alpha.-hydro-.omega.-hydroxypoly(oxyethylene)poly(oxypropylene)poly(oxyethylene) block copolymers, polyoxyethylene alkyl ethers, polyoxyethylene castor oil derivatives, other like material, derivatives thereof, and various combinations thereof, without limitation.

Suitable surfactants include, but are not limited to, mono and diglycerides of fatty acids and polyoxyethylene sorbitol esters, such as, Atmos 300 and Polysorbate 80, and pluronic acid, sodium lauryl sulfate, and the like.

Preferred surfactants include mono and diglycerides of fatty acids and polyoxyethylene sorbitol esters, such as, Atmos 300 and Polysorbate 80 in amounts ranging from about 0.01 to about 20%, preferably about 0.05 to about 12%, more preferably from about 0.1 to about 7% of the biologically compatible carrier and even more preferably from about 0.5 to about 5% of the biologically compatible carrier.

The sweeteners used in the biologically compatible carriers according to the present invention optionally comprise one or more selected from the group consisting of those well known in the art, including both natural and artificial sweeteners, other like material, derivatives thereof, and various combinations thereof, without limitation. Sweeteners and flavors need not be added to biologically compatible carriers intended for non-oral administration.

Suitable sweeteners include, but are not limited to, aspartame, acesulfame K, sucralose, sucrose, dextrose, maltodextrin, maltose, and the like.

Preferred sweeteners include aspartame, acesulfame K, sucralose, dextrose, maltodextrin, and maltose, in amounts ranging from about 0.01 to about 20%, preferably about 0.05 to about 115%, more preferably from about 0.1 to about 12% of the biologically compatible carrier and even more preferably from about 0.5 to about 10% of the biologically compatible carrier.

Any biologically compatible carrier can be used that is biologically compatible, providing it has adequate adhesion to the reagent formulations, exhibits the desired release profile, and has compatibility with the biologically active agents and any other components that may be present in the reagent formulation.

Preferred biologically compatible carriers according to the present invention optionally comprise a base solution that includes at least one component from each of the groups comprising: water soluble polymers, water, and fillers.

Typically, the base solution is prepared by adding an initial mixture of dry or wet components to water that is stirred.

To the base solution, additional components are added, such as antimicrobial agents, binding agents, coloring agents, cooling agents, emulsifying agents, fillers, flavoring agents, glycols, humectants, plasticizers, preservatives, release agents, saliva stimulating agents, stabilizers, starches, surfactants, sweeteners, water soluble polymers, water, other like material, derivatives thereof, and various combinations thereof, without limitation.

It should be appreciated that there is considerable overlap between the aforementioned components in common usage, since a given component is often classified differently by different practitioners in the field, or is commonly used for any of several different functions. Thus, the above listed components should be taken as merely exemplary, and not limiting, of the types of components that can be included in biologically compatible carriers of the present invention. The amounts of such components can be readily determined by one skilled in the art, according to the particular properties desired.

(ii) Biologically Compatible Control Codes

Preferably, the system of the present invention further comprises biologically compatible control codes.

Preferred biologically compatible control codes, according to the present invention optionally comprise various types of biologically compatible control codes, without limitation.

Any biologically compatible control code can be used that is biologically compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

Preferred biologically compatible control codes, according to the present invention optionally comprise at least one component from each of the groups further comprising coloring agents, emulsifying agents, flavoring agents, glycols, plasticizers, preservatives, surfactants, sweeteners, water soluble polymers, water, other like material, derivatives thereof, and various combinations thereof, without limitation.

Preferably, the system of the present invention further comprises biologically compatible control codes applied to the biologically compatible array corresponding to the sites on the biologically compatible array.

Preferably, the biologically compatible array, the reagent formulation, or the tamper resistant pack, or combinations thereof, comprise suitably compatible control codes that comprise at least one graphic image per site.

Preferably, the biologically compatible array, the reagent formulation, or the tamper resistant pack, or combinations thereof, comprise suitably compatible control codes that further comprise at least one graphic image per site that further comprise barcodes.

Preferably, the biologically compatible array, the reagent formulation, or the tamper resistant pack, or combinations thereof, comprise suitably compatible barcodes that are machine readable, by barcode scanners or the equivalent.

Preferably, the biologically compatible array, the reagent formulation, the tamper resistant pack, or combinations thereof, comprise suitably compatible control codes to indicate which biologically active agents are to be emplaced on the biologically compatible carrier.

Preferably, the biologically compatible array, the reagent formulation, the tamper resistant pack, or combinations thereof, comprise suitably compatible control codes to indicate where the biologically active agents are to be emplaced on the biologically compatible carrier.

Additionally, the biologically compatible control codes optionally provide a reliable and effective production control system in that the user can effectively emplace the proper biologically active agents at each site on the biologically compatible array by comparing the biologically compatible control codes at each site with a master record. The user can prevent production errors and thus assure that the correct biologically active agents are emplaced at each site on the biologically compatible array through the use of barcode scanning devices which scan the biologically compatible control codes during the production methodology.

The method of the present invention thus comprises, in an alternate embodiment, devices which scan the biologically compatible control codes during the production methodology. Such devices may include barcode scanners, without limitation. It is well known in the art that improved levels of production errors and improved production monitoring are thereby realized.

Further, the biologically compatible control codes optionally provide a reliable and effective feedback system in that the user can determine if the proper administrations have been taken on the proper days and at the proper times by comparing the biologically compatible control codes with a master record. The user or monitoree can effectively monitor and determine if an administration has been missed, prevent improper administrations, or administrations at improper times.

Preferably, the biologically compatible array, the reagent formulation, the tamper resistant pack, or combinations thereof, comprise biologically compatible control codes to that can effectively monitor and verify whether a user has administered the correct biologically active agents.

Preferably, the biologically compatible array, the reagent formulation, the tamper resistant pack, or combinations thereof, comprise biologically compatible control codes to that can effectively monitor and verify whether a user has administered the correct biologically active agents at the correct time.

The application of the system of the present invention is further specified in the application section below.

It should be appreciated that there is considerable overlap between the aforementioned components in common usage, since a given component is often classified differently by different practitioners in the field, or is commonly used for any of several different functions. Thus, the above listed components should be taken as merely exemplary, and not limiting, of the types of components that can be included in biologically compatible carriers of the present invention. The amounts of such components can be readily determined by one skilled in the art, according to the particular properties desired.

(iii) Biologically Compatible User Instructions

Preferably, the system of the present invention further comprises biologically compatible user instructions.

Preferred biologically compatible user instructions, according to the present invention optionally comprise various types of biologically compatible user instructions, without limitation.

Any biologically compatible user instruction can be used that is biologically compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

Preferred biologically compatible user instructions, according to the present invention optionally comprise at least one component from each of the groups further comprising coloring agents, emulsifying agents, flavoring agents, glycols, plasticizers, preservatives, surfactants, sweeteners, water soluble polymers, water, other like material, derivatives thereof, and various combinations thereof, without limitation.

Preferably, the system of the present invention further comprises biologically compatible user instructions applied to the biologically compatible array corresponding to the sites on the biologically compatible array.

Preferably, biologically compatible user instructions comprise the sites, another portion of the biologically compatible array, and combinations thereof, without limitation.

Additionally, the biologically compatible user instructions provide a feedback system in that the user can determine and confirm proper administration by comparing the biologically compatible user instructions with a calendar or clock. This helps assure administration is performed on the proper days and at the proper times. The user has a means that provides a degree of feedback in determining if an administration has been missed, an improper administration taken, or an administration taken at an improper time.

It is noted that any user dependent feedback system relying solely on user instruction as described herein or otherwise disclosed, is limited to the level of skill of the user and it thus subject to errors and misuse, caused by factors referenced above. (See Libow et al., Parkin et al., Seidl et al., and Schwartz et al.)

Preferably, the biologically compatible array, the reagent formulation, or the tamper resistant pack, or combinations thereof, include suitably compatible user instructions to indicate when the administration is to be taken.

Preferably, at least the top of at least one site is transparent or translucent and the site color is used as the biologically compatible user instruction.

Preferably the site color is used to indicate when to take the administration although it will be appreciated by those skilled in the art that other biologically compatible user instructions may also apply such as different administrations or biologically active agents.

Preferably, the biologically compatible array, the reagent formulation, or the tamper resistant pack, or combinations thereof, as a whole or in part, such as a site or sites, row or rows, column or columns, are colored. Preferably the color coding is part of a logical progression such as a rainbow color spectrum, traffic light range, or other culturally familiar range of colors to show an appropriate order of use or consumption.

Preferably the color coding comprises the biologically compatible array, the reagent formulation, the tamper resistant pack, or combinations thereof, without limitation.

Preferably, the range of colors comprises the biologically compatible array, the reagent formulation, or the tamper resistant pack, or combinations thereof, and further comprises a recognizable sequence.

Preferably, biologically compatible user instructions comprise a key defining or explaining the color coding. For example, morning sites are colored yellow corresponding to the rising sun, and evening sites are colored orange corresponding to the setting sun, or other colors and combinations thereof, without limitation.

Preferably, a color key is provided on the biologically compatible array to indicate which color corresponds with which administration date or time. The color key comprises suitably compatible user instructions and may be provided on the biologically compatible array, directly on the sites, the tamper resistant pack, and combinations thereof, without limitation.

Preferably, time biologically compatible user instructions may be incorporated into the biologically compatible array of the present invention. The time biologically compatible user instructions may be of any type, without limitation. The time biologically compatible user instructions correspond to at least two distinct time periods, but may correspond to any number of distinct time periods without limitation. For example, without limitation, the time biologically compatible user instructions may indicate a general time of the day corresponding to each of the sites or a specific time of the day corresponding to each of the sites. Time suitably compatible user instructions may be indicated directly on the sites, another portion of the biologically compatible array, the tamper resistant pack, and combinations thereof, without limitation.

Preferably, time biologically compatible user instructions include markings selected from the group consisting of: AM, PM, morning, day, daytime, afternoon, evening, night, nighttime, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 37, 38, 39, 40, 41, 42, 43, 44, 45, 45, 46, 47, 48, 49, 50, 51, 52, 53, 54, 55, 56, 57, 58, 59, 60, One, Two, Three, Four, Five, Six, Seven, Eight, Nine, Ten, Eleven, Twelve, Thirteen, Fourteen, Fifteen, Sixteen, Seventeen, Eighteen, Nineteen, Twenty, Twenty One, Twenty Two, Twenty Three, Twenty Four, Twenty Five, Twenty Six, Twenty Seven, Twenty Eight, Twenty Nine, Thirty, Thirty One, Thirty Two, Thirty Three, Thirty Four, Thirty Five, Thirty Six, Thirty Seven, Thirty Eight, Thirty Nine, Forty, Forty One, Forty Two, Forty Three, Forty Four, Forty Five, Forty Six, Forty Seven, Forty Eight, Forty Nine, Fifty, Fifty One, Fifty Two, Fifty Three, Fifty Four, Fifty Five, Fifty Six, Fifty Seven, Fifty Eight, Fifty Nine, Sixty, and the like, and combinations thereof, without limitation.

Preferably, day biologically compatible user instructions are incorporated into the biologically compatible array of the present invention. The day biologically compatible user instructions may be of various types, without limitation. The day biologically compatible user instructions correspond to at least two distinct sites. For example, without limitation, the day biologically compatible user instructions may be a specific day of the week, or an abbreviation of said day, a specific date, or a general succession of days. Day suitably compatible user instructions may be indicated directly on the sites, another portion of the biologically compatible array, the tamper resistant pack, and combinations thereof, without limitation.

Preferably, day biologically compatible user instructions include markings selected from the group consisting of: Monday, Tuesday, Wednesday, Thursday, Friday, Saturday, Sunday, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and the like, and combinations thereof, without limitation.

Preferably, week biologically compatible user instructions are incorporated into the biologically compatible array of the present invention. The week biologically compatible user instructions may be of various types, without limitation. The week biologically compatible user instructions correspond to at least two distinct sites. For example, without limitation, the week biologically compatible user instructions may be a week of the month, or an abbreviation of said week, a specific date, or a general succession of weeks. Week suitably compatible user instructions may be indicated directly on the sites, another portion of the biologically compatible array, the tamper resistant pack, and combinations thereof, without limitation.

Preferably, week biologically compatible user instructions include markings selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and the like, and combinations thereof, without limitation.

Preferably, month biologically compatible user instructions are incorporated into the biologically compatible array of the present invention. The month biologically compatible user instructions may be of various types, without limitation. The month biologically compatible user instructions correspond to at least two distinct sites. For example, without limitation, the month biologically compatible user instructions may be a month of the year, or an abbreviation of said month, a specific date, or a general succession of months. Month suitably compatible user instructions may be indicated directly on the sites, another portion of the biologically compatible array, the tamper resistant pack, and combinations thereof, without limitation.

Preferably, month biologically compatible user instructions include markings selected from the group consisting of: J, F, M, A, M, J, J, A, S, O, N, D, Jan, Feb, Mar, Apr, May, Jun, Jul, Aug, Sep, Oct, Nov Dec, January, February, March, April, May, June, July, August, September, October, November, December, and combinations thereof, without limitation.

Preferably, quarter biologically compatible user instructions are incorporated into the biologically compatible array of the present invention. The quarter biologically compatible user instructions may be of various types, without limitation. The quarter biologically compatible user instructions correspond to at least two distinct sites. For example, without limitation, the quarter biologically compatible user instructions may be a quarter of the year, or an abbreviation of said quarter, a specific date, or a general succession of quarters. Quarter suitably compatible user instructions may be indicated directly on the sites, another portion of the biologically compatible array, the tamper resistant pack, and combinations thereof, without limitation.

Preferably, quarter biologically compatible user instructions include markings selected from the group consisting of: 1,2,3,4, JFM, AMJ, JAS, OND, and combinations thereof, without limitation.

Biologically compatible user instructions can include the time, for example ‘AM’ or ‘PM’, the day, for example ‘Monday’ or ‘Tuesday’, the week number, for example ‘1’ or ‘2’, the month for example ‘January’ or ‘February’, or the quarter, for example ‘JFM’ or ‘AMJ’, or any other identifier, and combinations thereof, without limitation.

Preferably, biologically compatible user instructions comprise color, time, day, week, month, quarter, for each separate row or column, each site, each type of biologically active agent, each time, each day, each week, each month, each quarter, and combinations thereof, without limitation.

It should be appreciated that there is considerable overlap between the aforementioned components in common usage, since a given component is often classified differently by different practitioners in the field, or is commonly used for any of several different functions. Thus, the above listed components should be taken as merely exemplary, and not limiting, of the types of components that can be included in biologically compatible carriers of the present invention. The amounts of such components can be readily determined by one skilled in the art, according to the particular properties desired.

(iv) A plurality of Sequenced Sites of Varying Isolated Formulations of Biologically Active Agents

Preferably, the system of the present invention further comprises a plurality of sequenced sites of varying isolated formulations of biologically active agents.

Preferred sequenced sites, according to the present invention optionally comprise various types of sites, of various formulations, of various shapes, without limitation.

Any sequenced site can be used that is biologically compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

Preferred sites, according to the present invention comprise sites wherein one or more sites comprise varying biologically active agents and reagent formulations.

Preferably, the sites further comprise one or more types, uneven administrations, different administration concentrations, different ratios, different combinations, different volumes of biologically active agents in each site, and combinations thereof, without limitation.

Preferably, the sites further comprise varying biologically active agents that are organized and grouped for use of the biologically active agents in each site.

Preferably, the sites are grouped for sequential use. By way of example, sites are organized in a straight line, circle, arc, any other shaped line, and combinations thereof, without limitation.

Preferably, the sites include a group of sites organized in a plurality of rows. Sites are preferably designed to be organized in discrete and separate locations thus keeping each administration identifiable visually or tactily from all the other administrations.

Preferably, the sites further comprise any shape of site and the shape comprises the terms any portion, any spot, any dot, any random shape, any curvilinear shape, any geometric shape, and combinations thereof, without limitation.

In one embodiment, the total number of sites included on the biologically compatible array is for a seven day time period.

In an alternative embodiment, the total number of sites included on the biologically compatible array is for a fourteen day time period.

In another alternative embodiment, the total number of sites included on the biologically compatible array is for a twenty-eight day time period.

In another alternative embodiment, the total number of sites included on the biologically compatible array is for a thirty day time period.

In another alternative embodiment, the total number of sites included on the biologically compatible array is for a thirty-one day time period.

Preferably, the sites further comprise isolated administrations which are stable inside the sites until removed, preserving biologically active agents that may breakdown in air or in contact with other biologically active agents, so they remain stable until ready for use.

Preferably, the sites further comprise biologically active agents suitable for delivery into, onto, or near the target bodily cavity or area.

Preferably, the sites further comprise reagent formulations suitable for delivery into, onto, or near the target bodily cavity or area.

Preferably, the sites further comprise biologically active agents admixed with the reagent formulation within each site.

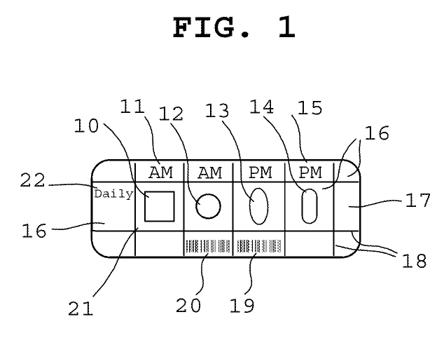

FIG. 1 shows a plan view of a novel sequenced biologically compatible delivery system in accordance with one embodiment of the present invention. The system shown in FIG. 1 generally indicated by arrow 16 contains one row of sites 17. Each site 21 contains a specific concentration and ratio of biologically active agents (‘the administration’). For example, each individual site 10 is an administration 21. In this embodiment, the system 16 contains four sites containing four administrations 10, 12, 13, and 14. The system 16 is divided into an AM section for morning administrations and a PM section for evening administrations. For a complete 1 day course of administrations 10, 12, 13, and 14, one system 17 is used.

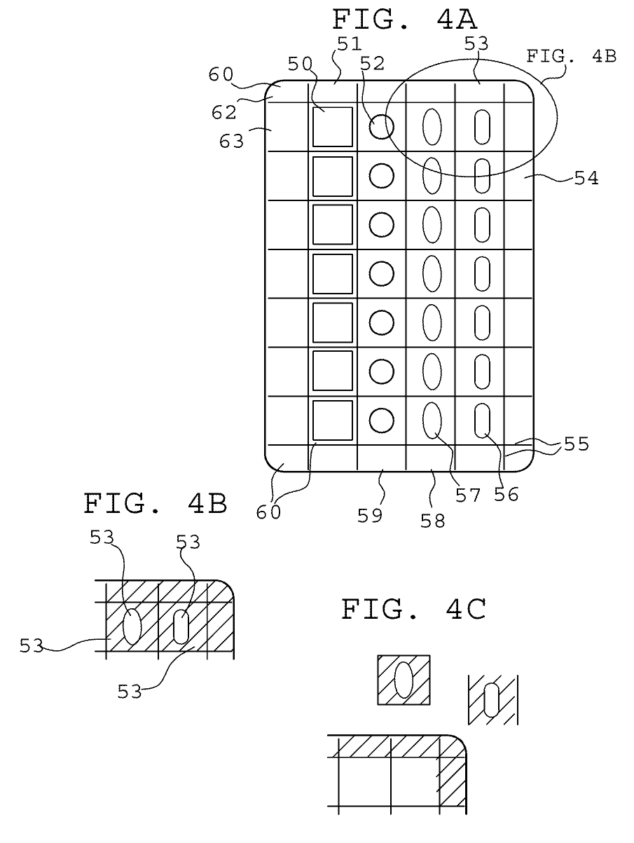

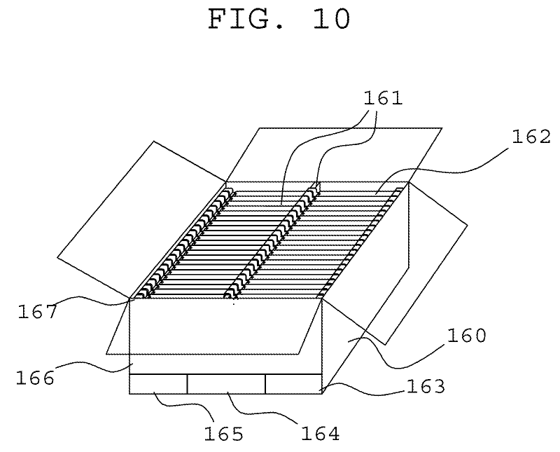

Administrations 10, 12, 13, and 14 contain incompatible biologically active agents. For example, administration 10 contains biologically active agents which are incompatible with biologically active agents in administration 12. Thus, within the row, the administrations in the four sites each contain incompatible biologically active agents. Perforations 18 are included around the sites 21 on the system 16 to allow the user to detach administrations 10, 12, 13, and 14. The system may further be incorporated into a tamper resistant pack shown in FIGS. 5A-5B. In addition, the system 16 may further be incorporated into an outer packing shown in FIG. 10. The biologically compatible array, tamper resistant pack and outer packing may have suitably compatible user instructions indicating to the user as to when to use each of the administrations 10, 12, 13, and 14.

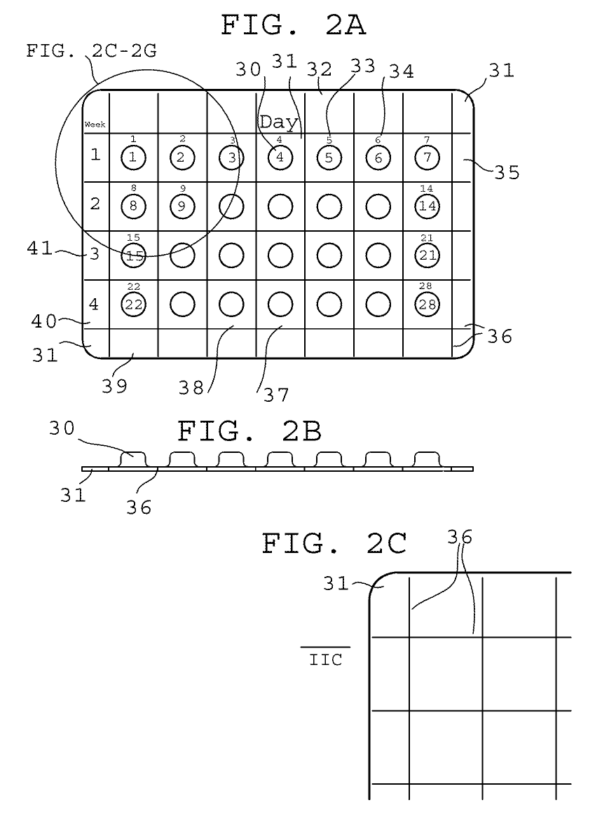

FIGS. 2A-2G show plan views of a novel sequenced biologically compatible delivery system in accordance with another embodiment of the present invention.

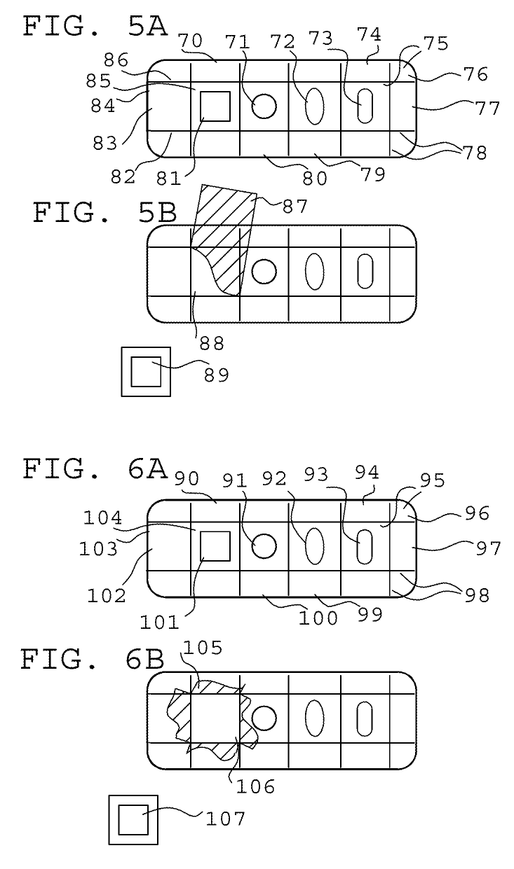

The system shown in FIG. 2A generally indicated by arrow 39 contains four rows 35 of sites 30. Each site 30 contains a specific concentration and ratio of biologically active agents (‘the administration’). Each individual site 30 is an administration 31. In this embodiment, the system 39 contains four rows of sites 35 containing seven administrations 31 corresponding to the number of days in a week. Perforations 36 are included around the sites on the system 39 to allow the user to detach each administration 31. For a complete 28 day course of administrations 31, one system 39 is used. The system 39 may further be incorporated into a tamper resistant pack as shown in FIGS. 5A-5B and FIGS. 6A-6B. In addition, the system 39 may further be incorporated into an outer packing as shown in FIG. 10. The biologically compatible array, tamper resistant pack and outer packing may have suitably compatible user instructions 41 indicating to the user as to when to use each site 30.

The system of FIG. 2A is shown in elevation in FIG. 2B showing sites 30, biologically compatible carrier 31, and perforations 36.

A portion of the system of FIG. 2A is shown in FIG. 2C showing biologically compatible carrier 31, and perforations 36.

A portion of the system of FIG. 2A is shown in FIG. 2D showing biologically compatible carrier 31, and biologically compatible user instructions 33. The administration columns 45 are bordered by a columns and rows of sequentially ordered day biologically compatible user instructions 33. Each day biologically compatible user instruction indicates a day of the week.

A portion of the system of FIG. 2A is shown in FIG. 2E showing biologically compatible carrier 31, and biologically compatible control codes 37, 38.

A portion of the system of FIG. 2A is shown in FIG. 2F showing biologically compatible carrier 31, and sites 30 and 42.

A portion of the system of FIG. 2A is shown in FIG. 2G showing biologically compatible array 31, site 30, an administration 43 separated from the biologically compatible array, and the empty area resulting from separation of administration 44.

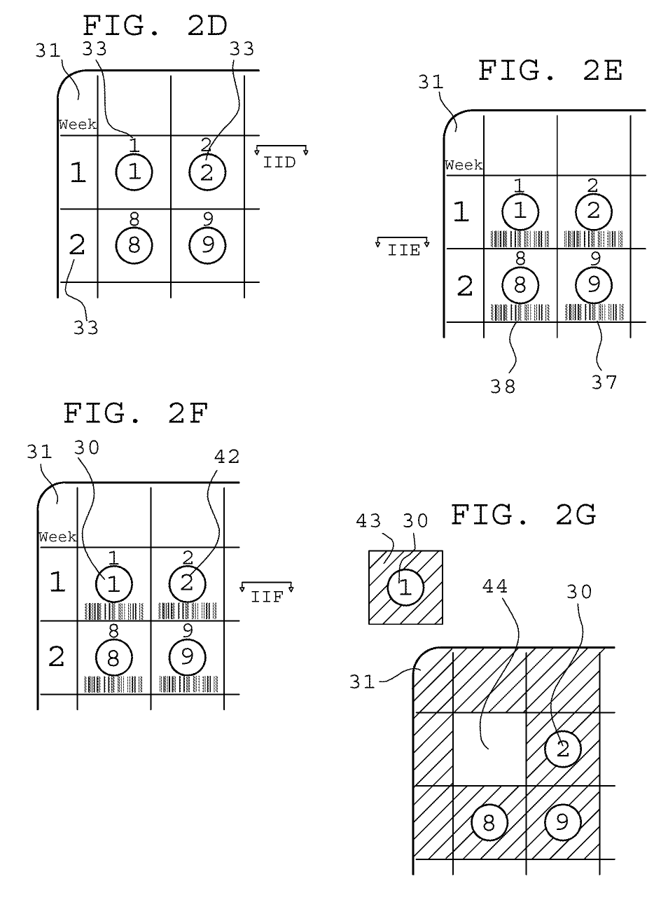

FIG. 3C is a cross-sectional side-view illustrating a portion of the biologically compatible carrier shown in FIG. 2C, looking through view IIC, showing biologically compatible carrier 31, and perforations 36.

FIG. 3D is a cross-sectional side-view illustrating a portion of the biologically compatible user instructions shown in FIG. 2D, looking through view IID, showing biologically compatible carrier 31, and biologically compatible user instructions 33.

FIG. 3E is a cross-sectional side-view illustrating a portion of the biologically compatible control codes shown in FIG. 2E, looking through view IIE, showing biologically compatible carrier 31, and biologically compatible control codes 37, 38.

FIG. 3F is a cross-sectional side-view illustrating a portion of the sites shown in shown in FIG. 2F, looking through view IIF, showing biologically compatible carrier 31, and sites 30 and 42.

FIG. 3G is a cross-sectional side-view illustrating one site separated from a portion of the biologically compatible array shown in FIG. 2G, showing biologically compatible array 31, site 30, an administration 43 separated from the biologically compatible array, and the empty area resulting from separation of administration 44.

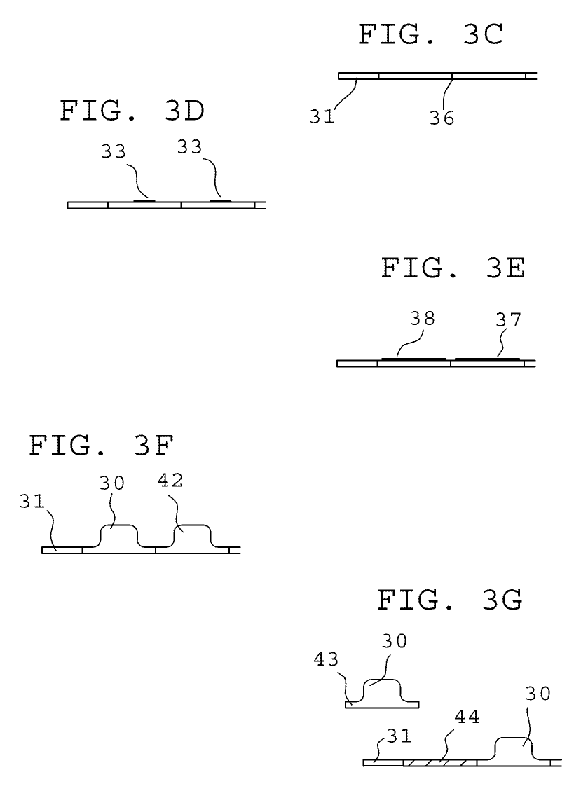

FIG. 4A shows a plan view of an alternate embodiment of the present invention. The system generally indicated by arrow 62 contains seven rows 54 of four sites 50 corresponding to a four times per day administration of biologically active agents. For example, each individual site 50 is an administration 67.

The site columns are bordered by a column of sequentially ordered day biologically compatible user instructions 61. The site rows are bordered by a row of sequentially ordered time biologically compatible user instructions 51, 53. Each day biologically compatible user instruction 61 indicates a day of the week. Time biologically compatible user instructions 51, 53 are provided on each site to show time of administration. The time biologically compatible user instruction 51, 53 indicate morning and evening. First and second columns 51 are used for the two morning administrations and third and fourth columns 53 are used for the two evening administrations.

Within each row, the administration has a site containing biologically active agents which are incompatible with the biologically active agents in the administrations in the other sites. Administrations 50, 52, 63, and 64 contain incompatible biologically active agents. For example, administration 50 contains biologically active agents which are incompatible with biologically active agents in administration 52. Administration 63 contains biologically active agents which are incompatible with biologically active agents in administration 64. Within the row, the administrations in the four sites each contain incompatible biologically active agents.

A portion of the system of FIG. 4A is shown in FIG. 4B showing sites 63, 64, corresponding to administrations 65, 66. The biologically active agents of administration 65 are incompatible with the biologically active agents of administration 66. Therefore, by storing administration 65 and administration 66 in separate sites 63 and 64, respectively, the incompatible agents of administration 65 and administration 66 are prevented from interacting with one another.

FIG. 4C shows the system 62 of FIG. 4A with administrations 65, 66 of FIG. 4B separated from the biologically compatible array, and the empty area resulting from the separation of administrations 65, 66. The administrations are separated by tearing the perforations 55 of FIG. 4A.

In a further embodiment, the morning sites are colored yellow corresponding to the rising sun, and evening sites are colored orange corresponding to the setting sun, or other colors and combinations thereof, without limitation.

In a further embodiment, the system may be in the shape of a roll with administrations in sequential order.

The present system, application, and method may incorporate uneven or unequal dosing of agents throughout an hourly, daily, weekly, monthly, or quarterly period of time. For example, the biologically compatible array may provide differing amounts of biologically active agents in the morning, compared to the evening. Moreover, the types of biologically active agents in the morning and evening need not be the same. Usage of biologically active agents may be alternating. For example, and without limitation, in every other administration, the amount of a particular vitamin may be the same, or in every other administration, the absence or presence of a particular vitamin alternates. Usage of biologically active agents may be progressive. For example, and without limitation, increasing amounts of a certain biologically active agents may be provided for administration as the plan progresses.

In an alternate embodiment, the sites comprise biologically agent agents well suited for the delivery via the mucous membranes of a target bodily cavity or area of a user, particularly the buccal mucosa. Biologically active agents that exhibit absorption problems due to solubility limitations, degradation in the gastrointestinal tract, or extensive metabolism, are particularly well suited.

The sites of the present invention may comprise any type of biologically active agents, without limitation.

Preferably, the biologically active agent comprises at least one biologically active agent selected from the group consisting of:

.alpha.-adrenergic agonists, .alpha.-adrenergnic blockers, .alpha.-glucosidase inhibitors, .beta.-adrenergic antagonists, .beta.-adrenergic blockers, beta.-blockers, 5-HT agonists, 5HT.sub.1 agonists, 5HT.sub.3 antagonists, abortifacients, ACE inhibitors, actives, acyclic nucleosides, adrenocorticotropic hormones, amino acids, amnestics, anabotic steroids, analgesic-antipyretics, analgesics, androgens, anesthetics, anorexics, anthelmintics, antiallergics, antialopecials, anti-anginal agents, antianginals, antianiebics, antiarrhythmics, antiarrhythmics, antiartbritics, antiasthmatics, anti-bacterial agents, anti-benign prostate hypertrophy agents, antibiotics, antibodies, anticholinergics, anti-coagulants, anticonvulsants, antidepressants, anti-depressants, antidiabetics, antidiarrheals, antidiuretics, antidotes, antidyskinetics, antiemetics, antiepileptics, antiestrogens, antifungals, antigens, antiglaucoma agents, antigout agents, antihelminthics, antihistaminics, antihyperglycemics, antihypertensives, anti-inflammatory agents, antimalarials, antimicrobial agents, antimigraine agents, antimigraines, antimuscarinics, antineoplastics, anti-obesity agents, anti-osteoporosis agents, antiparkinsons agents, antipheochromocytoma agents, antiplaque agents, antipneumocystis agents, antiprostatic hyperplasia agents,antiprotozoals, antipruritics, antipsoriatics, antipsychotics, antipyretics, antirickettsials, antispasmodics, antithrombocythemics, antithrombotics, anti-thyroid agents, antitumor agents, antitussive expectorants, antitussives, antiulceratives, anti-urinary incontinence agents, anti-viral agents, anxiolytics, arachidonic acid, aromatase inhibitors, awakening agents, barbiturates, B-endorphins, benzodiazepine antagonists, benzodiazepines, beta blockers, bile salt derivatives, bile salts, bioactive agents, bioflavonoids, biopharmaceuticals, bradycardic agents, bradykinins, bronchodilators, buffers, butyrophenones, calcium channel blockers, carbonic anhydrase inhibitors, cardiac inotropic agents, cardiotonics, cardiovascular-acting agents, central nervous system agents, cessation of smoking agents, chemotherapeutics, choleretics, cholinergics, cholinesterase inhibitors, cholinesterase reactivators, CNS function modifiers, CNS stimulants, cognition enhancers, corticosteroids, Cox-2 inhibitors, cytokine antagonists, cytoprotectants, decongestants, diuretics, dopamine receptor agonists, dopamine receptor antagonists, drugs, ectorarasiticides, emetics, enkephalins, enzymes, erectile dysfunction improvement agents, essential fatty acids, essential oils, estrogens, expectorants, fatty acids, fibrinogen receptor antagonists, fusion inhibitors, GABA stimulators, ganglionic stimulants, gastric secretion inhibitors, gastro-intestinal agents, gastroprokinetics, gene constructs, general nonselective CNS depressants, general nonselective CNS stimulants, glycoprotein 120 antagonists, gonadotropic hormones, growth hormones, H.sub.2-antagonists, H.sub.2-receptor antagonists, hemostatics, herbs, histamine H.sub.2 receptor antagonists, histamine receptor antagonists, hormones, hypnotics, hypotensive-acting agents, hypotensives, immunomodulators, immunosuppresants, immunosuppressants, inhibitors, integrase inhibitors, interferons, keratolyptics, keratolytics, leukotriene inhibitors, lipid regulating agents, local anesthetic agents, local antifungal agents, local antipruritic agents, local antisecretory agents, long-chained polyunsaturated fatty acids, macrolides, macromolecular agents, MAO inhibitors, minerals, monounsaturated fatty acids, mucolytics, muscle relaxants, mydriatics, narcotic analgesics, narcotic antagonists, narcotics, neuroleptics, neuromuscular blocking agents, non-essential fatty acids, non-narcotic analgesics, non-nucleoside reverse transcriptase inhibitors, non-steroidal anti-inflammatory agents, nootropics, nucleic acids, nucleoside reverse transcriptase inhibitors, nucleoside transport inhibitors, nucleosides, nutritional agents, nutritional oils, nutritional supplements, omega 3 fatty acids, omega 6 fatty acids, omega 9 fatty acids, opiod analgesics, opioid analgesics, opioid antagonists, oxytocics, parathyroid hormones, peptides, phencyclidines, polypeptides, polysaccharides, polyunsaturated fatty acids, potassium channel activators, progestins, protease inhibitors, proton pump inhibitors, psychoneurotropic agents, psychopharmacological drugs, renal excretion agents, renal vascular-acting agents, respiratory stimulants, ribonucleotide reductase inhibitors, salts, sedatives, serenics, serotonin receptor agonists, serotonin receptor antagonists, serotonin uptake inhibitors, sex hormones, steroids, stimulants, substituted phenols, thrombolytics, thyroid hormones, tocolytics, vasodilator-acting agents, vasodilators, vasoprotectants, vasopressors, vitamins, breath freshening compounds, flavors used for oral hygiene, fragrances used for oral hygiene, active agents used for oral cleansing, active agents used for dental cleansing, biologically acceptable salts, isomers, esters, solvates, derivatives thereof, genetically engineered derivatives thereof, hydrates thereof, precursors thereof, mixtures thereof, other like material, and various combinations thereof, without limitation.

Preferably, administrations may be prescription or non-prescription biologically agents, without limitation. The prescription biologically active agent may be a chemobiologically active agent, a cholesterol reducing agent, a contraceptive agent, a hormone replacement agent, a steroidal agent, a tobacco antagonistic agent, a weight loss agent, an algesic agent, an anti-infective agent, an appetite suppressing agent, an osteoporotic agent, and combinations thereof, without limitation.

Preferably, administrations may be incompatible biologically active agents comprising acidic agents and basic agents, agents requiring an anhydrous environment and agents requiring a non-anhydrous environment, effervescent agents and high water content reagent formulations or administration forms, gelatin reagent formulations and aldehyde containing reagent formulations, hydrophobic agents and hydrophilic agents, olefinic agents and non-olefinic agents, pH sensitive agents and non-pH sensitive agents, quaternary ammonium agents and anionic agents, and combinations thereof, without limitation.

The sites of the present invention may comprise any biologically active agents, without limitation.

Preferably, the biologically active agent comprises at least one biologically active agent selected from the group consisting of:

(−)-cis-1-(2-hydroxymethyl )-1,3-oxathiolane 5-yl)-cytosine(lamivudine), (−)-cis-4-[2-amino-6-(cyclopropylamino)-9H-purin-9-yl]-2-cyclopentene-1-m-ethanol (abacavir), (1-alpha. 2-beta. 3-alpha)-9-[2,3 bis(hydroxymethyl)cyclobutyl]guanine [(−)BHCG SQ-34514 lobucavir], (3S)-tetrahydrofuran-3-yl (1S,2R)-[[(4-aminophenyl)sulphonyl)](isobutyl)amino]-1-benzyl-2-(phosphon-ooxy)propylcarbamate monocalcium salt(fosamprenavir), (4S)-6-Chloro-4-[1E)-cyclopropylethenyl])-3,4-dihydro-4-(trifluoromethyl)-2(1H)-quinazolinone (DPC-083), (R)-[[2-(6-Amino-9H-purin-9-yl )-1-methylethoxy]methyl]phosphonic acid bis-(isopropoxy carbonyloxymethyl )ester (bis-POC-PMPA), (R)-N-tert-butyl-3-[(2S,3S)-2-hydroxy-3-N-[(R)-2-N-(isoquinolin-5-yloxyac-etyl)amino-3-methylthio-propanoyl]amino-4-phenylbutanoyl]-5,5-dimethyl-1,3-thiazolidine-4-carboxamide (KNI-272), (S)-1-(3-hydroxy-2-phosphonyl-methoxypropyl)cytosine (HPMPC), (S )-6-chloro4-(cyclopropylethynyl)-1.4-dihydro4-(trifluoromethyl)-2H-3,1-benzoxazin-2-one(efavirenz, DMP 266), 1-tetramethyl-4-propyl-11,12-dihydro-2H,6H. 10H-benzo(1.2-b:3.4-b′:5.6-b″)tripyran-2-one ((+)calanolide A), .alpha.-interferon, .alpha.-trichosanthin, [[(1R)-2-(6-amino-9H-purin-9-yl)-1-methylethoxy]methyl]phosphonic acid(tenofovir), [[[2-(6-amino-9H-purin-9-yl)ethoxy]methyl]phosphinylidene]bis(oxymethylen-e)-2,2-dimethylpropanoic acid (bis-POM PMEA adefovir dipivoxil), 1-(ethoxymethyl)-5-(1-methylethyl)-6-(phenylmethyl)-2,4(1H,3H)-pyrimidine-dione (MKC-442),1,1′-azbbis-formamide (ADA), 1,11-(1,4-phenylenebis(methylene))bis-1,4,8,11-tetraazacyclotetrad-ecane octahydrochloride (AMD-3100), 1,25-dihydroxycholecalciferol, 1,4-bis[3-[(2,4-dichlorophenyl)carbonylamino]-2-oxo-5,8-disodiumsulfanyl]-naphthalyl-2,5-dimethoxyphenyl-1,4-dihydrazone (FP-21399),1-[3-(isopropylamino)-2-pyridyl]4-[5-(methanesulfonamido)-1H-indol-2-ylca-rbonyl]piperazine monomethanesulfonate(delavirdine), 2′,3′-didehydrothymidine (d4T stavudine).(−)-beta-D-2, 2′,3′-dideoxyadenosine, 2′,3′-dideoxycytidine (ddC zalcitabine), 2′,3′-dideoxyinosine (ddI didanosine), 2′-deoxy-5-iodo-uridine(idoxuridine), 2-acetylpyridine 5-[(2-chloroanilino)thiocarbonyl)thiocarbonohydrazone hydroxyurea, 2-hydroxypropyl-.beta.-cyclodextrin, 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide(AG-1776), 3-[1-[3-[2-(5-trifluoromethylpyridinyl)-sulfonylamino]phenyl]propyl]4-hy-droxy-6alpha-phenethyl-6beta-propyl-5,6-dihydro-2-pyranone(tipranavir), 3′-azido-2′,3′-dideoxythymidine-5′-H-phosphophonate(phosphonovir), 3′-azido-3′-deoxythymidine (AZT zidovudine), 3′-deoxy-3′-fluorothymidine, 4R-(4alpha.5alpha.6beta)]-1,3-bis[(3-aminophenyl)methyl]hexahydro-5,6-dih-ydroxy-4,7-bis(phenylmethyl)-2H-1,3-diazepin-2-one dimethanesulfonate(mozenavir), 5-(3,5-dichlorophenyl)thio-4-isopropyl-1-(4-pyridyl)methyl-1H-imidazol-2-ylmethyl carbamate(capravirine), 5-chloro-2′,3′-dideoxy-3′-fluorouridine, 6-diaminopurine dioxolane (DAPD), 7-dehyrdocholesterol, 9-[(2R,3R,4S)-3,4-bis(hydroxymethyl)-2-oxetanosyl]adenine(oxetanocin-G), 9-[4-hydroxy-2-(hydroxymethyl )but-1-yl]-guanine(H2G), ABT-606 (2HM-H2G), acarbose, acetaminophen, acetazolamide, acetretin, acetyl cysteine, acetylcholine chloride, acrivastine, activated charcoal, acyclic nucleosides, acyclovir, adefovir dipivoxil, adriamycin, aerobid/nasolide, alatrofloxacin, albendazole, albuterol, alcohol, alendronate, alexidine, alfentanil, alglucerase, alpha-((2-acetyl-5-methylphenyl)amino)-2.6-dichloro-benzeneacetamide (loviride), alpha-linolenic acid, alpha-tocopherol, Aluminium, aluminum hydroxide, amantadine hydrochloride, amantadine, ambenomium, amifostine, amiloride hydrochloride, amino acids, aminocaproic acid, aminoglutethimide, aminophylline, amiodarone, amlodipine, amphetamine, amphotericin B, amprenavir, amyl nitrate, angelica, angiotensin I, antihemophilic factor (human), antihemophilic factor (porcine), antihemophilic factor (recombinant), aprotinin, arachidonic acid, arsenic, ascorbic acid, asparaginase, aspirin, atenolol, atorvastatin, atovaquone, atracurium besylate, atropine, azatadine meleate, azidothymidine, azithromycin, azmacort, AZT, aztreonam, bacitracin, baclofen, baptisia, BCG vaccine, BCNU, becalermin, beclomethasone diproprionate, beclomethasone, beconase, belladona, benezepril, benzodiazepines, benzoic acid, benzonatate, benzquinamide, benzyl alcohol, bepridil hydrochloride, beta-carotene, betamethasone, bicalutaminde, bicalutanide, bicarbonate carbonate, biotin, blenoxane, bleomycin sulfate, bleomycin, boron, bovine growth hormone, bretylium, bromide, bromine, brompheniramine maleate, brompheniramine, budesonide, bupropion, busulfan, butenafine, cadmium, caffeine, cajeput oil, calcifediol, calcipotriene, calcitonin human, calcitonin salmon, calcitonin, calcitriol, calcium apatite, calcium carbonate, calcium citrate-malate, calcium gluconate, calcium hydroxide, calcium lactate, calcium levulinate, calcium oxide, calcium phosphate, calcium sulfate, calcium, calendula, camptothecin, capecitabine, capreomycin sulfate, capsaicin, captopril, caramiphen edisylate, carbamazepine, carbamezepine, carbidopa, carbinoxamine maleate, carbohydrates, carbonyl iron, carboplatin, carfentanil, carotenes, catechu black, cefamole nafate, cefazolin sodium, cefepime hydrochloride, cefixime, cefonicid sodium, cefoperazone, cefotaxime, cefotetan disodium, cefoxitin sodium, ceftizoxime, ceftriaxone, cefuroxime axetil, celecoxib, cephalexin, cephalosporins, cephapirin sodium, cerivastatin, cerubidine, cesartan, cetirizine, cetyl pyridium chloride, chenodeoxycholate, chlophedianol , chloride, chlorides, chlorine, chlorpheniramine maleate, chlorpheniramine, chlorpromazine, cholecalciferol, cholecaliferol, cholera vaccine, cholesterol, choline, chorionic gonadotropin, chromium, cidofovir, cilostazol, cimetidine, cinnarizine, ciprofloxacin, cis-1-(2-(hydroxymethyl)-1,3-oxathiolan-5-yl)-5-fluorocytosine, cisapride, cisplatin, citric acid, cladribine, clarithromycin, clemastine fumarate, clemastine, clidinium bromide, clindamycin, clodronate, clofibrate, clomiphene, clomipramine, clonidine dopamine, clonidine, clopidogrel, clotrimazole, clozapine, cobalt, codeine, coenzyme Q10, colestipol, colistimethate sodium, colistin sulfate, conjugated estrogens, conjugated linolenic acid, copper, corticotropin, cosmegen, cosyntropin, crataegus, cromolyn sodium, cyclobenzaprine, cyclophosphamide, cyclosporin, cytarabine, cytoxan, dactinomycin, dalteparin sodium, danaparoid, danazol, dantrolene, daunorubicin, deanol, decadron, dehydrocholate, dehydroepirosterone, denileukin diftitox, desferrioxamine, desmopressin acetate, desmopressin, desogestrel, dexamehasone, dexbrompheniramine, dexchlorpheniramine maleate, dexchlorpheniramine, dextromethorphan hydrobromide, dextromethorphan, diatrizoate meglumine diatrizoate sodium, diazepam, diclofenac, dicoumarol, dicyclomine, didanosine, diflunisal, digoxin, dihydroergotamine, dihydrotachysterol, diltiazem, dimethyl sulfoxide, diphenhydramine citrate, diphenhydramine hydrochloride, diphenhydramine, diphenylpyraline hydrochloride, diprivan, dipyridamole, dirithromycin, docosahexanoic acid, docosapentaenoic acid, docusate sodium, domiphen bromide, donezepil, dopamine hydrochloride, dornase alpha, doxacurium chloride, doxorubicin, doxylamine succinate, droperidol, dyphylline, echinacea (Coneflower), EDTA, efavirenz, eicosapentaenoic acid, enalapril, enalaprilat, enkephalin, enoxaparin sodium, enoxaparin, ephedrine, epinephrine, epoetin alpha, eprosartan, ergocalciferol, ergoloid mesylates, ergonovine, ergotamine, erythromycin, erythropoetin, esmolol hydrochloride, esmolol, esomeprazole, essential fatty acid sources, essential fatty acids, esterified estrogens, estradiol, estropipate, ethanol, ethinyl estradiol, ethosukimide, etidronate disodium, etodolac, etomidate, etoposide, etylpyridinium chloride, eucalyptus oil, factor IX, famciclovir, famotidine, fennel oil, fenofibrate, fenoprofen calcium, fentanyl, ferrous fumarate, fexofenadine, finasteride, flavin adenine dinucleotide, flavin mononucleoride, flavin mononucleotide, flonase, flovent, fluconazole, fludarabine, flunisolide, fluorides, fluorine, fluoxetine, flurbiprofen, fluticasone proprionate, fluvastatin, folic acid, foscamet sodium, fosphenytoin, frovatriptan, furazolidone, furosemide, gabapentin, gamma-linolenic acid, ganciclovir, gemfibrozil, gentamycin, geranium oil, girofle oil, glibenclamide, glimepiride, glipizide, glucagon, gluteraldehyde, glyburide, glycerrhiza (Glycerrhiza glabra), glycochenocholate, glycopyrolate, GnRH, gonadorelin, gonadotropin releasing hormone synthetic analogs thereof, granulocyte colony stimulating factor, granulocyte macrophage colony stimulating factors, grepafloxacin, griseofulvin, guaifenesin, haemophilus B conjugate vaccine, halofantrine, haloperidol, heparin sodium, heparin, hepatitis A virus vaccine inactivated, hepatitis B virus vaccine inactivated, heroin, hexabarbital, hydrochloride, hydrocodone, hydrodeoxycholate, hydromorphone, Hypericum (Hypericaceae perforatus), ibuprofen, ifosfamide, imipramine, indamycin, indarubicin, indinavir sulfate, indinavir, indomethacin, influenza virus vaccine, inositol, insulin aspart, insulin detemir, insulin glargine, insulin lispro, insulin NPH, insulin procine, insulin, insulin-human, interferon alpha, interferon beta, interleukin II, interleukin-2, interleukin-3, iodine, ipecac, ipratropium bromide, irbesartan, irinotecan, iron, isoetharine, isoproterenol HCL, isosorbide dinitrate, isosorbide, isotretinoin, itraconazole, ivermectin, japanese encephalitis virus vaccine, ketamine, ketoconazole, ketoprofen, ketorolac tromethamine, ketorolac, konicin chloride soap, krameria, labetalol, lamivudine, lamotrigine, lansoprazole, laurocapram, layer oil, lecithin, leflunomide, lemon oil, leucovorin calcium, leuprolide acetate, levodopa, levofloxacin, levonorgestrel, levorphanol, lidocaine, lincomycin, linoleic acid, lisinopril, lithium, lobucavir, lofentanil, lomefloxacin, loperamide famotidine, loperamide, loracarbef, loratadine, lorazepam, lormetazepam, losartan, lovastatin, L-thryroxine, lutein, lycopene, lypressin, magnesium carbonate, magnesium hydroxide, magnesium oxide, magnesium stearate, magnesium sulfate, magnesium, mallow sage, manganese, mannitol, matricaria, measles virus vaccine, meclizine, meclofenamate, medroxyprogesterone, mefloquine, megestrol acetate, menadione, menaquinone, meningococcal vaccine, menotropins, menthol, mepenzolate bromide, meprobamate, mesalamine, metaproterenol, mefformin hydrochloride, methadone, methdone, methenamine, methohexital, methotrexate, methotrimeprazine, methoxsalen, methscopolamine, methsuximide, methyltestosterone, methysergide, metoclopramide, metolazone, metopon, metoprolol, metronidazole, mezocillin sodium, miconazole, midazolam, mifepristone, miglitol, minoxidil, mitomycin, mitoxantrone, mivacurium chloride, molybdenum, mometasone furoate, montelukast, morphine, mumps viral vaccine, mutamycin, myristic acid, myrrh, myrte oil, N-(2(R)-hydroxy-1(S)-indanyl)-2(R)-phenyl-methyl-4(S)-hydroxy-5-(1-(1-(4-benzo[b]furanylmethyl)-2(S)-N′-(tert-butylcarboxamido)piperazinyl)pentanam-ide (MK-944A), N′-[2(S)-Hydroxy-3(S)-[N-(methoxycarbonyl)-1-tert-leucylamino]-4-phenylbu-tyl-N.sup.alpha-(methoxycarbonyl)-N′-[4-(2-pyridyl)benzyl]-L-tert-leucylhy-drazide(BMS-232632), nabumetone, N-acetylcysteine (NAC), nadolol, nalbuphine, nalorphine, naloxone, naltrexone, naphthoquinone, naproxen, naratriptan, nedocromil sodium, nelfinavir, neostigmine bromide, neostigmine methyl sulfate, neurontin, nevirapine (BI-RG-587), niacin, niacinamide, nicotinamide adenine dinucleotide, nicotinamide, nicotine, nicotinic acid, nickel, nifedepine, nifedipine, nilsolidipine, nilutanide, nitrofurantoin, nitroglycerin, nitroprusside, nizatidine, n-methyl pyrrolidone, norethindrone acetate, norethindrone, norfloxacin, norgestimate, norgestrel, nystatin, obtained spearmint oil, octonidine, octreotide acetate, oestradiol, ofloxacin, oil of cinnamon, oleic acid, olpadronate, omeprazole, oprevelkin, oregano oil, oxaliplatin, oxaprozin, oxazepam, oxtriphylline, oxymorphone, oxytocin, paclitaxel, palmitic acid, pamidronate disodium, pancuronium bromide, pantethine, pantothenic acid, paracalcitol, paraplatin, paroxetine, penciclovir, penicillins, pentagastrin, pentamidine isethionate, pentazocine, pentobarbital, pentostatin, pentoxifylline, pentylenetetrazol, perfloxacin, periciclovir, phenacemide, phenacetin, phenelzine, pheneturide, phenobarbital, phensuximide, phentolamine mesylate, phenyhydantoin, phenylalanine, phenylbutazone, phenylepherine, phenylpropanolamine, phosphonoformic acid, phosphorus, phylloquinone, physostigmine salicylate, phytolaca, picrotoxin, pine oil, pioglitazone, piperacillin sodium, piroxicam, pizofetin, plague vaccine, plantago (Plantago major), platelet derived growth factor, platinol, pneumococcal vaccine polyvalent, poleythylene glycol its derivatives, poliovirus vaccine (inactivated), poliovirus vaccine live (OPV), polyethylene oxide, polymyxin B sulfate, polyoxyethylene-9-lauryl ether, polysaccharide iron, polyvinyl alcohol, polyvinyl pyrrolidone, potassium iodide, potassium, pralidoxime chloride, pramlintide, pranlukast, pravastatin, prednisolone, pregabalin, primidone, PRO-2000, PRO-542, probenecid, probucol, prochlorperazine, procysteine, progesterone, promethazine hydrochloride, propafenone, propanidid, propanolol, propantheline bromide, propofol, propolis, propranolol, propylene glycol, prostaglins, proteins, pseudoephedrine hydrochloride, pseudoephedrine sulfate, pseudoephedrine, pulmicort, pyridostigmine bromide, pyridostigmine, pyridoxine (Vitamin B6), pyrilamine maleate, quaternary ammonium salts, quinapril, rabeprazole, rabies vaccine, raloxifene, ranitidine, rapamycin, recombinant human growth hormones, repaglinide, reserpine, residronate, reticulose (Product-R), retinal, retinoic acid, retinol, rhinocort, ribavarin, ribavirin, riboflavin, ribonucleotide reductase inhibitors, rifabutine, rifapentine, rimantadine hydrochloride, rimexolone, ritanovir, ritonavir, rizatriptan, rofecoxib, rosemary oil, rosiglitazone, rosmarinus, rotavirus vaccine, rubex, S-1360, salmeterol xinafoate, salvia, salycilates, sanguinarine, saquinavir, sarriette oit, scopolamine, selenium, sertraline, sibutramine, sildenafil citrate, silicon, simvastatin, sincalide, sirolimus, small pox vaccine, sodium bicarbonate, sodium carbonate, sodium cholate,sodium deoxycholate, sodium dodecyl sulfate, sodium glycocholate, sodium glycodeoxycholate, sodium lauryl sulfate, sodium lithocholoate chenocholate, sodium stearate, sodium taurocholate, sodium, solatol, somatostatin, sotalol, sparfloxacin, spectinomycin, spironolactone, stavudine, stearic acid, streptokinase, streptozocin, strychnine, styrax, sucralfate, sufentanil, sulfur, suloctidil, sulthiame, sumatriptan, suxamethonium chloride, tacrine hydrochloride, tacrine, tacrolimus, tamoxifen, tamsulosin, targretin, taurochenocholate, taurochenodeoxycholate, taurodeoxycholate, taxol, tazarotene, tea-tree oil, telmisartan, teniposide, terazosin, terbenafine, terbinafine, terbutaline sulfate, terbutaline, terpin hydrate, testosterone, tetrahydrocannabinol, theophylline, thiamin pyrophosphate, thiamin, thiamine, thiamylal, thiopental, thiopeta, thioridazine, thiotepa, thyme oil, thymosin, tiagabine, ticarcillin, ticlopidine, tiludronate, timolol, tirofibran, tissue type plasminogen activator, tizanidine, TNFR:Fc, TNK-tPA, tocopherol, tocotrienol, tolmetin sodium, topiramate, topotecan, toremifene, toremitfene, tramadol, tranylcypromine, tretinoin, triamcinolone acetonide, triazolam, triclosan, trimethadione, trimethobenzamide, trimetrexate gluconate, tripelennamine citrate, triprolidine hydrochloride, troglitazone, trolapril, trospectinomycin, trovafloxacin, tryptophan, tsuga, tubocurarine chloride, tumor necrosis factor, typhoid vaccine live, ubidecarenone, unofficial B Vitamins, urea, urokinase, ursocholate, ursodeoxycholate, valaciclovir, valacyclovir, valsartan, vanadium, vancomycin, varicella virus vaccine live, vasopressin, vecuronium bromide, venlafaxine, verteporfin, vigabatrin, vinblastine, vincristine, vinorelbine, vitamin A, vitamin B complex, vitamin B6, vitamin B1, vitamin B12, vitamin B2, vitamin B3, vitamin B6, vitamin C, vitamin D, vitamin E, vitamin K, vitamin P, vumon, warfarin sodium, xycodone, yellow fever vaccine, zafirlukast, zalcitabine, zanamivir, zidovudine, zileuton, zinc compounds, zinc, zolendronate, zolmitriptan, zolpidem, zopiclone, biologically acceptable salts, isomers, esters, solvates, derivatives thereof, genetically engineered derivatives thereof, hydrates thereof, precursors thereof, mixtures thereof, other like material, and various combinations thereof, without limitation.

The amount of agent to be incorporated into the administration, according to the present invention, depends on the kind of agent and is usually between 0.01 and 20%, but it can be higher if necessary to achieve the desired effect.

The amount of agent that can be used in the administration, according to the present invention, is also dependent upon the dose needed to provide an effective amount of the agent. Examples of specific doses for exemplary biologically active agents that can be delivered per one site are reviewed in Table A.

TABLE A
BIOLOGICALLY ACTIVE AGENT PREFERRED DOSE

| | | |
|---|---|---|
| Acrivastine | 8 | mg. |
| Azatadine Maleate | 1 | mg. |
| Brompheniramine Maleate | 4 | mg. |
| Chlorpheniramine Maleate | 4 | mg. |
| Dextromethorphan Hydrobromide | 30 | mg. |
| Dexchlorpheniramine | 2 | mg. |
| Diphenhydramine Hydrochloride | 25 | mg. |
| Famotidine | 10 | mg. |
| Ketoprofen | 25 | mg. |
| Loperamide | 2 | mg. |
| Loratidine | 10 | mg. |
| Nicotine | 2 | mg. |
| Phenylephrine Hydrochloride | 10 | mg. |
| Pseudoephedrine Hydrochloride | 30 | mg. |
| Sumatriptan Succinate | 70 | mg. |
| Triprolidine Hydrochloride | 2.5 | mg. |
| Zolmitriptan | 2.5 | mg. |

Non-limiting exemplary prescription agents include adefovir, adriamycin, aerobid, albuterol, alendronate, amprenavir, atorvastatin, azidothymidine, azmacort, AZT, BCNU, beclomethasone diproprionate, beconase, blenoxane, bleomycin, budesonide, carbidopa, carboplatin, cephalosporins, cerubidine, chlorpromazine, cisplatin, clofibrate, clonidine, colestipol, conjugated estrogens, cosmegen, cyclophosphamide, cytoxan, dactinomycin, daunorubicin, decadron, desogestrel, dexamehasone, diazepam, diltiazem, dipivoxil, doxorubicin, ergoloid mesylates, erythromycin, esterified estrogens, estradiol, estropipate, ethinyl estradiol, flovent, flonase, flunisolide, fluticasone proprionate, indamycin, indarubicin, levodopa, levonorgestrel, losartan, meclizine, medroxyprogesterone, meprobamate, methotrexate, methyltestosterone, mitomycin, mometasone furoate, mutamycin, nasolide nifedepine, norethindrone acetate, norethindrone, norgestimate, norgestrel, oxaliplatin, paclitaxel, paraplatin, penicillins, platinol, propranolol, pulmicort, quinapril, raloxifene, rhinocort, rubex, sotalol, streptozocin, tamoxifen, taxol, teniposide, terbutaline, theophylline, thioridazine, thiotepa, triamcinolone acetonide, vinblastine, vumon, zidovudine, other like material, and various combinations thereof, without limitation.

The non-prescription agent can be a vitamin or derivative thereof, or a mineral compound or derivative thereof, or a nutritional compound or derivative thereof. The vitamin or mineral compound or nutritional compound may be 1.25-dihydroxycholecalciferol, 7-dehydrocholesterol, alpha-tocopherol, ascorbic acid, beta-carotene, biotin, calcium apatite, calcium carbonate, calcium citrate-malate, calcium gluconate, calcium hydroxide, calcium lactate, calcium levulinate, calcium oxide, calcium phosphate, calcium sulfate, calcium, carbonyl iron, chloride, chromium, copper, docusate sodium, ferrous fumarate, flavin adenine dinucleotide, flavin mononucleoride, folic acid, iodine, magnesium carbonate, magnesium hydroxide, magnesium oxide, magnesium stearate, magnesium sulfate, magnesium, menadione, menaquinone, molybdenum, naphthoquinone, niacin, niacinamide, nicotinamide adenine dinucleotide, nicotinamide, nicotinic acid, pantothenic acid, phosphorus, phylloquinone, polysaccharide iron, potassium, retinal, retinoic acid, retinol, riboflavin, sodium, sulfur, thiamin pyrophosphate, thiamin, tocopherol, tocotrienol, tryptophan, zinc, other like material, and various combinations thereof, without limitation. Derivatives of vitamin compounds include salts, alkaline salts, esters and chelates of any vitamin compound, without limitation. The nonprescription agent can also be an herbal compound, herbal extract, other like material, derivative thereof, and various combinations thereof, without limitation.

Incompatible agents may be any biologically active agents that may not be formulated together in a single administration or stored together in direct contact because the biologically active agents will be adversely effected and also any biologically active agents that cannot be formulated together in a single administration because the resulting total of the administration amounts would result in a single administration which is too large. Incompatible agents also include those biologically active agents which would otherwise be stored in direct contact, however, one of the biologically active agents is preferably formed in a manner which is either not preferred or incompatible with the other biologically active agents. Incompatible agents may include two or more biologically active agents wherein at least one biologically active agent is a prescription agent and at least one biologically active agent is a non-prescription agent. The incompatible agent may include any incompatible agent, other like material, derivative thereof, and various combinations thereof, without limitation.

Non-limiting exemplary incompatible agents include, without limitation, activated charcoal and amyl nitrate, ascorbic acid and aluminum hydroxide, ascorbic acid and sodium bicarbonate, citric acid and sodium carbonate, etylpyridinium chloride and sodium stearate, folic acid and calcium carbonate, gelatin capsules and formaldehyde, gelatine capsules and gluteraldehyde, konicin chloride and soap, other like material, derivative thereof, and various combinations thereof, without limitation.

It should be appreciated that there is considerable overlap between the aforementioned components in common usage, since a given component is often classified differently by different practitioners in the field, or is commonly used for any of several different functions. Thus, the above listed components should be taken as merely exemplary, and not limiting, of the types of components that can be included in biologically compatible carriers of the present invention. The amounts of such components can be readily determined by one skilled in the art, according to the particular properties desired.

(v) Reagent Formulations Supporting Effective Delivery of and Admixed with the Biologically Active Agents

Preferred reagent formulations, according to the present invention optionally comprise various types of reagent formulations, of various formulations, of various shapes, without limitation.

Any reagent formulation can be used that is biologically compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

Preferably, the system of the present invention further comprises reagent formulations supporting effective delivery of and admixed with the biologically active agents.

Any reagent formulation can be used that is biologically compatible, providing it has adequate adhesion to biologically compatible carrier, exhibits the desired disintegration profile, and has compatibility with the biologically active agents and any other components that may be present in the biologically compatible array.

Preferred reagent formulations, according to the present invention optionally comprise various types of reagent formulations, without limitation.

Preferred reagent formulations, according to the present invention optionally comprise at least one component which is a liquid, semi-liquid, semi-solid, solid, releasable liquid, slowly releasable liquid, releasable semi-liquid, slowly releasable semi-liquid, releasable semi-solids, slowly releasable semi-solids, releasable solids, slowly releasable solids, other like material, derivatives thereof, and various combinations thereof, without limitation.

Preferred reagent formulations, according to the present invention optionally further comprise components each provided in a form which facilitates mixing, such as a dry powder. This provides for convenient combination of the components, even if they happen to be insoluble or otherwise inappropriate for aqueous combination.

Preferred reagent formulations, according to the present invention optionally further comprise components, incipients or inactive components on the GRAS list (‘generally regarded as safe’).

Preferred reagent formulations, according to the present invention optionally further comprise at least one physical state which is a liquid, semi-liquid, semi-solid, solid, releasable liquid, slowly releasable liquid, releasable semi-liquid, slowly releasable semi-liquid, releasable semi-solids, slowly releasable semi-solids, releasable solids, slowly releasable solids, cream, gel, suspension, mixture, other like material, derivatives thereof, and various combinations thereof, without limitation. For example, reagent formulations according to the present invention include liquids, solids, pastes, gums, taffies, creams, other like material, derivatives thereof, and various combinations thereof, without limitation.

For example, creams may be made using a cream base, for example DMS base cream and oils such as castor oil. In addition, the reagent formulation often will be a liquid, semi-liquid, semi-solid, or solid, or with an encapsulation coat on the reagent formulation which acts as a shell surrounding and encapsulating a liquid, semi-liquid, cream, semi-solid, or solid reagent formulation.

Preferred reagent formulations, according to the present invention optionally further comprise a base reagent formulation. Biologically active agents may then be added to the base as required to form the administration.

The reagent formulations can be formed of any of the biologically active agents, disintegrants, and other optional additives described herein.

Preferred reagent formulations, according to the present invention comprise biologically active agents, disintegrants and optionally at least one additive.

Preferred disintegrants used in the reagent formulations according to the present invention optionally comprise one or more selected from the groups consisting of acrylic acid polymers, alginates, biosynthetic gums, biosynthetic process starches, cellulose ethers, cellulosic compounds, cellulosic materials, edible polymers, gelatins, glycols, hydrocolloid flours, land plant extracts, modified starches, natural fiber extracts, natural gums, natural plant exudates, natural seaweeds, natural seed gums, polyacrylates, polyethylene oxides, seaweed extracts, starches, starch derivatives, vinyl polymers, other like material, derivatives thereof, and various combinations thereof, without limitation.

The disintegrants used in the reagent formulations according to the present invention comprise one or more selected from the group consisting of acacia gum, agar gum, algin gum, alginic acid, amylose, arabic gum, calcium alginate, carboxymethylcellulose, carboxymethylethyl cellulose, carboxyvinyl polymer, carrageenan, casein, cellulose acetate, cellulose nitrate, cellulose acetate butyrate, cellulose acetate trimellitate, cellulose acetate phthalate, chitin, chitosan, collagen, croscarmellose sodium, crosslinked gelatin, crosslinked polyvinylpyrrolidone, cross-linked carboxylicmethylcellulose, dextran, dextrin, elsinan, ethylcellulose, gelatin, ghatti gum, gluten, guar gum, high amylose starch, hydroxyethylcellulose, hydroxyethylmethylcellulose, hydroxypropylated high amylose starch, hydroxypropylcellulose, hydroxypropylmethylcellulose, hydroxypropylmethyl cellulose phthalate, levan, locust gum, methylcellulose, methylmethacrylate copolymer, natural fiber extracts, natural plant exudates, natural seaweeds, natural seed gum, pectin, polyacrylic acid, polyethylene glycol, poly(lactide coglycolide), polyethylene, polyvinyl alcohol, polyvinylpyrrolidone, pullulan, sodium alginate, sodium carboxymethylcellulose, sodium starch glycolate, soy protein isolate, tara gum, tragacanth gum, whey protein isolate, xanthan gum, zein, other like material, derivatives thereof, and various combinations thereof, without limitation.

Suitable disintegrants include, but are not limited to, Carbopol®, which may be obtained from B.F. Goodrich, Crospovidone or Polyplasdone® XL, which may be obtained from GAF, Croscarmelose or Ac-di-sol®, which may be obtained from FMC, Gantrez®, which may be obtained from GAF., Sentry Polyox®, available from Union Carbide, Methocel® and Klucel®, both available from the Dow Chemical Company, sodium carboxymethyl starches or Explotab®, which may be obtained from Edward Medell Co., Inc., hydroxyethyl cellulose, carboxymethyl cellulose, sodium carboxymethyl cellulose, methyl cellulose, ethyl cellulose, cellulose acetate phthalate, cellulose acetate butyrate, and the like.

The reagent formulation may comprise two or more suitable disintegrants in combination, for example, a carbomer combined with a polyethylene oxide, or a cellulosic compound combined with a starch, in an approximately 1:5 to 5:1 ratio.

Any disintegrant can be used that is biologically compatible, providing it has the required adhesion, disintegration profile, and compatibility characteristics.

The disintegrants comprise amounts ranging from about 0.01 to about 99%, preferably about 20 to about 80%, more preferably from about 35 to about 70% of the reagent formulations and even more preferably from about 40 to about 65% of the reagent formulations.

It is more preferred that the present reagent formulations contain only biologically active agent and disintegrant. It may be desirable in to include one or more additives in the reagent formulations, however, it must be emphasized that such additives are optional depending on processing, storage, and administration requirements.

Preferred reagent formulations, according to the present invention optionally comprise at least one additional additive from each of the groups further comprising adhesives, adsorbents, anti-adherents, anticoagulants, antifoaming agents, antioxidants, binders, buffers, chelating agents, coagulants, colorants, cooling agents, cryoprotectants, emulsifying agents, fillers, flavoring agents, glycols, humectants, hydrogen bonding agents, ion-exchange resins, lubricants, natural or synthetic waxes, permeation enhancers, plasticizers, polyols, preservatives, releasing agents, saliva stimulating agents, shellacs, solubilizers, solvents, stabilizers, starches, surfactants, sweeteners, thickeners, other like material, derivatives thereof, and various combinations thereof, without limitation.

The additives can be contained in an encapsulation coat in reagent formulations, or can be part of the reagent formulations. Suitable additives are commonly utilized to facilitate the processes involving the preparation of the reagent formulation, the encapsulation coating, or the administration. The additive can be pre-coated or encapsulated. Appropriate additives are well known in the art, and based on the functionality, examples are as follows.

Suitable additives include anticoagulants, such as acetylated monoglycerides and the like, without limitation.

Further, suitable additives include antifoaming agents, such as long-chain alcohols, silicone derivatives, and the like, without limitation.

Further, suitable additives include antioxidants, such as BHT, BHA, gallic acid, propyl gallate, ascorbic acid, ascorbyl palmitate, 4-hydroxymethyl-2,6-di-tert-butyl phenol, tocopheryl, and the like, without limitation.

The binding agents used in the reagent formulations according to the present invention optionally comprise one or more selected from the group consisting of dry binders, film binders, and chemical binders, without limitation. Examples of dry binders are dry starch, dry sugars, starch, gelatin and sugars such as sucrose, dextrose, molasses, and lactose. Examples of film binders are celluloses, hydroxypropylmethylcellulose, ethylcellulose, or other suitable cellulose derivatives. Examples of chemical binders are sugar syrups, corn syrup, gums, water soluble polysaccharides such as acacia, tragacanth, guar and alginates, gelatin, gelatin hydrolysate, agar, sucrose, dextrose, and non-cellulosic binders, such as vinyl pyrrolidone copolymers, PVP, PEG, starch paste, pregelatinized starch, sorbitol, and glucose. Other exemplary binders include povidone, acrylic and methacrylic acid co-polymers, pharmaceutical glaze, milk derivatives, whey, conventional binders, and those for compressed administrations which enhance adhesion and include starch, gelatin and sugars such as sucrose, dextrose, molasses, and lactose.

Suitable binding agents include, but are not limited to, starch, gelatin, maltodextrin, sucralose, and sugars such as sucrose, dextrose, molasses, lactose, and the like.

Preferred binding agents are maltodextrin, sucralose, and dextrose, in amounts ranging from about 0.01 to about 99%, preferably about 0.1 to about 70%, more preferably from about 1 to about 50% of the reagent formulations and even more preferably from about 1 to about 25% of the reagent formulations.

A buffering agent may also be desirable to place the administration in a favorable pH environment in the target bodily cavity or area. For example, passage across the mucosal tissues of the mouth, pharynx, and esophagus, without limitation. In addition, the reagent formulations of the present invention optionally further comprise the free acid form or the free base form of certain biologically active agents to buffer those biologically active agents such that extremes in pH, and resulting poor taste, are prevented. Buffering agents incorporated within the reagent formulations can be used to effect a pH change in the salival environment of the mouth in order to favor the existence of a unionized form of the active component or biologically active agent which more readily moves through the mucosal tissues administration wherein such is desirable.

In addition, pH adjustment can aid in producing a more palatable product with biologically active agents which are either severely acidic (and thus sour) or severely basic (and thus bitter). As a result, a buffer system such as citric acid and sodium citrate has been found to be desirable in the reagent formulation. A phosphate buffer system may alternatively be used.

The buffers used in the reagent formulations according to the present invention optionally comprise one or more selected from the group consisting of those for the biologically acceptable acid, such as acetic acid, acrylic acid, adipic acid, alginic acid, alkanesulfonic acid, amino acids, ascorbic acid, benzoic acid, butyric acid, carbonic acid, citric acid, boric acid, hydrochloric acid, hydrobromic acid, hydriodic acid, sulfuric acid, nitric acid, phosphoric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and for the biologically acceptable base, such as an aluminum hydroxide, ethylenediamine, ethanolamine, amino acid, an amino acid ester, ammonium hydroxide, potassium hydroxide, sodium hydroxide, sodium hydrogen carbonate, calcium carbonate, magnesium hydroxide, magnesium aluminum silicate, synthetic aluminum silicate, synthetic hydrotalcite, magnesium aluminum hydroxide, diisopropylethylamine, triethanolamine, triethylamine, triisopropanolamine, or for the salt of a biologically acceptable cation such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, other like material, derivatives thereof, and various combinations thereof, without limitation.

Further, suitable additives include chelating agents, such as EDTA and EDTA salts, and the like, without limitation.

Further, suitable additives include coagulants, such as alginates and the like, without limitation.

Further, suitable additives include coolants, such as monomenthyl succinate, Ultracool II, and the like, without limitation.

The components and additives described herein may comprise a white powder. Therefore, additional coloring is necessary if a colored end product is desired. Colorants can be added to the reagent formulations to facilitate administration and produce a desirable color. Coloring may also be important as a code, key, or legend to indicate the type, concentration of biologically active agent contained within a particular reagent formulation, or the order of administration of the sites.

The coloring agents used in the reagent formulations according to the present invention optionally comprise one or more selected from the group consisting of pigments such as titanium dioxide, iron oxides, silicates, sulfates, magnesium hydroxide and aluminum hydroxide, natural food colors and dyes suitable for food, drug and cosmetic applications, including FD&C Blue No. 2, Green No. 3, Blue No. 1, Red No. 40, Yellow No. 6, natural vegetable colorants, other like material, derivatives thereof, and various combinations thereof, without limitation. A description of FD&C dyes may be found in the Kirk-Othmer Encyclopedia of Chemical Technology. (See Kirk-Othmer) Any type of color known to be ‘FD&C’ certified or ‘GRAS’ may be used to provide coloring to the product.

Suitable coloring agents include, but are not limited to, titanium dioxide, FD&C Blue No. 2, Green No. 3, Blue No. 1, Red No. 40, Yellow No. 6, and the like.

Preferred coloring agents are Blue No. 1, Red No. 40, and Yellow No. 6, in amounts ranging from about 0.001 to about 5%, preferably about 0.0.005 to about 4%, more preferably from about 0.01 to about 2% of the reagent formulations and even more preferably from about 0.1 to about 1% of the reagent formulations.

Further, suitable additives include cryoprotectants, such as trehelose, phosphates, citric acid, tartaric acid, gelatin, dextran and mannitol, and the like, without limitation.

The emulsifying agents used in the reagent formulations according to the present invention optionally comprise one or more selected from the group consisting of triethanolamine stearate, quaternary ammonium compounds, acacia, gelatin, lecithin, bentonite, veegum, other like material, derivatives thereof, and various combinations thereof, without limitation.

Suitable emulsifying agents include, but are not limited to, triethanolamine stearate, quaternary ammonium compounds, acacia, gelatin, lecithin, and the like.

A preferred emulsifying agent is gelatin, in amounts ranging from about 0.01 to about 99%, preferably about 1 to about 70%, more preferably from about 2 to about 50% of the reagent formulations and even more preferably from about 5 to about 25% of the reagent formulations.

An enzyme inhibitor may also be desirable to reduce enzymatic degradation, when the active components or addtives are subject to enzymatic degradation. (See Bernskop-Schnurch, A.)

Preferred reagent formulations, according to the present invention optionally comprise at least one enzyme inhibitor from each of the groups further comprising inhibitors that are based on amino acids and modified amino acids, inhibitors that are not based on amino acids, other inhibitors such as peptides and modified peptides, polypeptide protease inhibitors, mucoadhesive polymers, polymer-inhibitor conjugates, other like material, derivatives thereof, and various combinations thereof, without limitation. Several examples of suitable enzyme inhibitors are set forth below.

Suitable enzyme inhibitors include inhibitors that are not based on amino acids, such as P-aminobenzamidine, FK-448, camostat mesylate, sodium glycocholate.

Further, suitable enzyme inhibitors include amino acids and modified amino acids, such as aminoboronic acid derivatives and n-acetylcysteine.

Further, suitable enzyme inhibitors include peptides and modified peptides, such as bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, and amastatin.

Further, suitable enzyme inhibitors include polypeptide protease inhibitors, such as aprotinin (bovine pancreatic trypsin inhibitor), Bowman-Birk inhibitor and soybean trypsin inhibitor, chicken egg white trypsin inhibitor, chicken ovo-inhibitor, and human pancreatic trypsin inhibitor and complexing agents, such as EDTA, EGTA, 1,10-phenanthroline and hydroxychinoline.

In addition, suitable enzyme inhibitors include mucoadhesive polymers and polymer-inhibitor conjugates, such as polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bowman-Birk inhibitor.

The choice and levels of the enzyme inhibitor are based on toxicity, specificity, and efficacy. The inhibitor can be suspended or dissolved in the base reagent formulation, added to the reagent formulation filler, or co-administered.

The following concepts describe the action of an inhibitor, acting solely or with other inhibitors and additives. Inhibitors can act as a complexing agent due to loss in enzymatic activity caused by deprivation of essential metal ions out of the enzyme structure. Inhibitors can also act as a competitive inhibitor, binding at the binding site of the enzyme and preventing the access. An inhibitor can also act as a non-competitive inhibitor, which can be sequentially bound to the enzyme site. As these are exemplary theories, this specification is not bound by any theory.

Suitable fillers for compressed administrations, without limitation, are dicalcium phosphate dehydrate, or Di-Tab®, which may be obtained from Stauffer (A-Tab), sugars that have been processed by cocrystallization with dextrin, or co-crystallized sucrose and dextrin such as Di-Pak®, which may be obtained from Amstar, spray-dried lactose, hydrolyzed starches, directly compressible starch, sucrose, sucrose-based materials, powdered sucrose, sorbitol, mannitol, dextrose, lactone, dry starch, and inorganics, such as dicalcium phosphate, hydroxyapitite, tricalcium phosphate, kaolin, calcium phosphate, talc, or titania, and cellulose, and sodium chloride.

The fillers used in the reagent formulations according to the present invention also optionally comprise one or more selected from the group consisting of silicon dioxide, magnesium stearate, potassium chloride, citric acid, dibasic calcium phosphate, hydroxyapitite, aluminum silicate, calcium carbonate, calcium sulfate, clay, dicalcium phosphate, gelatin, ground limestone, gum, magnesium carbonate, magnesium silicate, monocalcium phosphate, talc, titanium dioxide, tricalcium phosphate, other like material, derivatives thereof, and various combinations thereof, without limitation.

A preferred filler is calcium carbonate, in amounts ranging from about 0.01 to about 99%, preferably about 1 to about 70%, more preferably from about 2 to about 50% of the reagent formulations and even more preferably from about 5 to about 25% of the reagent formulations.

Preferably, the reagent formulation provides a palatable administration. A wide range of flavors are available for preparing palatable and desirable administrations of the present invention. These mask the taste of the biologically active agent, if such is unpalatable. Flavorings may be combined, as desired, to produce a particular flavor mix, as preferred for a particular administration.

The flavoring agents used in the reagent formulations according to the present invention optionally comprise one or more selected from the group consisting of natural and artificial flavors, spray-dried flavors, other like material, derivatives thereof, and various combinations thereof, without limitation. Sweeteners and flavors need not be added to reagent formulations intended for non-oral administration.

The flavorings used in the reagent formulations according to the present invention optionally comprise one or more selected from the group consisting of anise, apricot, banana, blackberry, blueberry, butter rum, butterscotch, caramel, cherry, chocolate, citrus oil, citrus, clove oil, clove oils, coconut, coffee, cranberry, ethyl vanillin, eucalyptol, fruit essence, fruit punch, grape, grapefruit, honey, lemon, licorice, mango, marshmallow, melon, menthol, methyl salicylate, mint, mint oil, mocha, orange, peach, peppermint oil, raspberry, spearmint, spearmint oil, strawberry, thymol, artificial vanilla, vanilla cream, vanilla, watermelon, wintergreen oil, zinc gluconate, other like material, derivatives thereof, and various combinations thereof, without limitation.

Each of these exemplary flavorings is obtainable in a concentrated liquid or concentrated powder form. Any number of flavorings may be combined in any desired ratio in order to produce the specific desired taste characteristics required for any particular application. For example, flavor combinations may be varied in order to be compatible with the flavor characteristics of any specific biologically active agent.

Appropriate flavoring components can be admixed to mask or optimize flavor perception in order to achieve acceptance of the administration.

Suitable flavoring agents include, but are not limited to, any of the aforementioned flavoring agents, and the like.

Preferred flavoring agents are any of the aforementioned flavoring agents, in amounts ranging from about 0.01 to about 40%, preferably about 0.5 to about 30%, more preferably from about 2 to about 25% of the reagent formulations and even more preferably from about 5 to about 10% of the reagent formulations.

For some applications, it is preferable to add a flavor enhancer to the reagent formulations in order to achieve a palatable product. Flavor enhancers provide a more pleasant taster sensation during the administration. Flavor enhancers, according to the present invention include materials such as ribotide (a nucleotide) and monosodium glutamate (‘msg’).

Suitable glycols include, but are not limited to, ethylene glycol, diethylene glycol, propylene glycol, and the like.

A preferred glycol is propylene glycol, in amounts ranging from about 0.1 to about 25%, preferably about 0.5 to about 20%, more preferably from about 1 to about 15% of the reagent formulations and even more preferably from about 2 to about 10% of the reagent formulations.

Suitable humectants include, but are not limited to, glycerol, propanediol, and the like.

A preferred humectant is glycerol, in amounts ranging from about 0.01 to about 50%, preferably about 1 to about 40%, more preferably from about 2 to about 30% of the reagent formulations and even more preferably from about 2 to about 10% of the reagent formulations.

Further, suitable additives include hydrogen bonding agents, such as magnesium oxide.

Further, suitable additives include ion-exchange resins, such as styrene/divinyl benzene copolymers, and quaternary ammonium compounds.

For some applications, it is preferable to add a permeation enhancer to the reagent formulations in order to improving the biologically active agent permeability across the mucosal membrane. Permeation enhancers are particularly important when nonlipophilic biologically active agents are used, but may be valuable for lipophilic biologically active agents as well.

Suitable permeation enhancers improve the mucosal membrane permeability to lipophilic and nonlipophilic drugs. Thus, the system, application, and method, according to the present invention optionally further comprise the use of lipophilic as well as nonlipophilic biologically active agents.

Suitable permeation enhancers include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate,sodium dodecyl sulfate, dimethyl sulfoxide, sodium lauryl sulfate, salts and other derivatives of saturated and unsaturated fatty acids, surfactants, bile salt analogs, derivatives of bile salts, polyethylene glycol monolaurate, glycerol monolaurate, lecithin, cholic acid, taurocholic acid, bile salt type enhancers, Tergitol®, Nonoxynol-9® and TWEEN-80®, or synthetic permeation enhancers, other like material, derivatives thereof, and various combinations thereof, without limitation.

Thus, the system, application, and method, according to the present invention optionally further comprise the use of lipophilic as well as nonlipophilic biologically active agents.

For some applications, it is preferable to add a plasticizer to the reagent formulations in order to control the solubility of the formulation. Plasticizers may be hydrophobic as well as hydrophilic in nature. Water-insoluble hydrophobic substances, such as diethyl phthalate, diethyl sebacate and caster oil are used to delay the release of water-soluble vitamins, such as vitamin B6 and vitamin C. In contrast, hydrophilic plasticizers are used when water-insoluble vitamins are employed which aid in dissolving the reagent formulations, making channels in the surface, which aid in nutritional release.

The plasticizers used in the reagent formulations according to the present invention optionally comprise one or more selected from the group consisting of citrate esters such as triethyl citrate, acetyl triethyl citrate, acetyltributyl citrate and phthalate esters such as diethyl phthalate, dibutyl phthalate and polyols, such as mannitol, xylitol, sorbitol or cyclodextrin, and polyethylene glycol esters, acetylated monoglycerides, and corn syrup, diethyl sebacate, dibutyl seccate, dibutyl sebacate, cronotic acid, propylene glycol, butyl phthalate, castor oil, glycerin, triacetin, glycerin, hydrogenated starch hydrolysate, maltitol, polyethylene glycol, propylene glycol, other like material, derivatives thereof, and various combinations thereof, without limitation.

Suitable plasticizers include, but are not limited to, corn syrup, glycerin, maltitol, sorbitol, and the like.

Preferred plasticizers are maltitol and corn syrup, in amounts ranging from about 0.01 to about 20%, preferably about 0.02 to about 15%, more preferably from about 0.03 to about 12% of the reagent formulations and even more preferably from about 0.5 to about 10% of the reagent formulations.

The preservatives used in the reagent formulations according to the present invention optionally comprise one or more selected from the group consisting of ascorbic acid, boric acid, benzoic acid, and salts thereof, benzalkonium chloride, benzyl alcohol, chlorocresol, methyl hydroxybenzoate, parabens, phenols, potassium sorbate, propyl hydroxybenzoate, salts of edentate, sodium benzoate, sorbic acid, quaternary ammonium compounds other like material, derivatives thereof, and various combinations thereof, without limitation.

Methods for evaluating the efficacy of preservatives are known to those skilled in the art.

Preferred preservatives are sodium benzoate and potassium sorbate in amounts from about 0.001% to about 5%, preferably from about 0.01% to about 1%.

Suitable preservatives include, but are not limited to, sodium benzoate, potassium sorbate, benzalkonium chloride, chlorocresol, and the like.

Preferred preservatives include sodium benzoate and potassium sorbate, in amounts ranging from about 0.0001 to about 5%, preferably about 0.005 to about 4%, more preferably from about 0.002 to about 3% of the reagent formulations and even more preferably from about 0.01 to about 1% of the reagent formulations.

In certain administrations, it may also be desirable to add a releasing agent in order to release the site. Releasing agents may also provide a degree of water resistance and may include substances such as compritol 888 (glyceryl behenate), calcium stearate, and sodium stearate. Releasing agents may enhance dissolution or they may inhibit dissolution as necessary, although as will be appreciated by those skilled in the art, modulating the particle size of the components in the reagent formulation and/or the density can provide a similar effect, providing improved manufacturability, optimization of erosion and dissolution rates, without any releasing agent.

Other releasing agents include anti-adherents (anti-sticking agents, glidants, flow promoters, lubricants) such as talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000, magnesium lauryl sulfate, magnesium stearate, calcium stearate, sodium stearylfumarate, talc, hydrogenated vegetable oils, polyethylene glycol, other like material, derivatives thereof, and various combinations thereof, without limitation.

Releasing agents will represent on the order of 0.01 wt. % to about 2 wt. %, preferably about 0.01 wt. % to 0.5 wt. %, of the administration.

Suitable release agents include, but are not limited to, magnesium stearate, glycerol monolaurate, compritol 888 (glyceryl behenate), calcium stearate, and the like.

A preferred release agent is glycerol monolaurate, in amounts ranging from about 0.001 to about 15%, preferably about 0.004 to about 10%, more preferably from about 0.08 to about 5% of the reagent formulations and even more preferably from about 0.01 to about 2% of the reagent formulations.

The saliva stimulating agents used in the reagent formulations according to the present invention optionally comprise one or more selected from the group consisting of citric, lactic, malic, succinic, ascorbic, adipic, fumaric, tartaric acids, other like material, derivatives thereof, and various combinations thereof, without limitation.

Suitable saliva stimulating agents include, but are not limited to, citric, lactic, malic, ascorbic, adipic, tartaric acids, and the like.

Preferred saliva stimulating agents are citric, malic, and ascorbic acids, in amounts ranging from about 0.01 to about 30%, preferably about 0.5 to about 20%, more preferably from about 1 to about 10% of the reagent formulations and even more preferably from about 2.5 to about 5% of the reagent formulations.

For some applications, it is preferable to add a solubilizer to the reagent formulations in order to increase the solubility of the biologically active agent or other components in the reagent formulations. The solubilizers used in the reagent formulations according to the present invention optionally comprise one or more selected from the group consisting of alcohols and polyols, such as ethanol, isopropanol, butanol, benzyl alcohol, ethylene glycol, propylene glycol, butanediols, glycerol, pentaerythritol, sorbitol, mannitol, transcutol, dimethyl isosorbide, polyethylene glycol, polypropylene glycol, polyvinylalcohol, cyclodextrins, cyclodextrin derivatives, and esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone, .delta.-valerolactone.beta.-butyrolactone, dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), triacetin, triethylcitrate, ethyl oleate, ethyl caprylate, dimethylacetamide, N-methylpyrrolidone, N-hydroxyethylpyrrolidone, polyvinylpyrrolidone, hydroxypropyl cyclodextrins, ethanol, glycofurol, transcutol, dimethyl isosorbide, and amides, such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide, and polyvinylpyrrolidone, and ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide), other like material, derivatives thereof, and various combinations thereof, without limitation.

Mixtures of solubilizers are also according to the present invention. Except as indicated, these compounds are readily available from standard commercial sources.

Preferred solubilizers are sorbitol, glycerol, triacetin, ethyl alcohol, PEG-400, glycofurol, propylene glycol, in amounts ranging from about 0.01 to about 50%, preferably about 0.05 to about 25%, more preferably from about 0.1 to about 20% of the reagent formulations and even more preferably from about 1 to about 15% of the reagent formulations.

Further, suitable additives include solvents, such as alcohols, ketones, esters, chlorinated hydrocarbons, water, ethanol, isopropyl alcohol, methylene chloride

The stabilizers used in the reagent formulations according to the present invention optionally comprise one or more selected from the group consisting of acacia gum, agar gum, algin gum, arabic gum, carrageenan, chitosan , ghatti gum, guar gum, pectin, tara gum, tragacanth gum, locust gum, xanthan gum, other like material, derivatives thereof, and various combinations thereof, without limitation.

Suitable stabilizers include, but are not limited to, agar gum, carrageenan, guar gum, xanthan gum, locust gum, and the like.

Preferred stabilizers are carrageenan, guar gum, locust gum, and xanthan gum, in amounts ranging from about 0.01 to about 20%, preferably about 0.02 to about 15%, more preferably from about 0.05 to about 10% of the reagent formulations and even more preferably from about 0.1 to about 5% of the reagent formulations.

The starches used in the reagent formulations according to the present invention optionally comprise one or more selected from the group consisting of acid and enzyme hydrolyzed corn and potato starches; any of several water-soluble polymers derived from a starch (e.g., corn starch, potato starch, tapioca starch) such as by acetylation, halogenation, hydrolysis (e.g:, such as which an acid), or enzymatic action; any type of water-soluble modified starch, including but not limited to oxidized, ethoxyolated, cationic, lypophilic and pearl starch; starches; polysaccharide-based derivatives, such as maltodextrin and maltodextrin derivatives, dextrates, cyclodextrin and cyclodextrin derivatives, maltodextrins including Maltrin® M100, Maltrin®M180, Maltrin® QD M550, and Maltrin® QD M600, all of which may be obtained from Grain Processing Corporation; and Pure-Cote® B792 modified corn starch, also available from Grain Processing Corporation, other like material, derivatives thereof, and various combinations thereof, without limitation.

Suitable starches include, but are not limited to, maltodextrins including Maltrin® M100, Maltrin® M180, Maltrin® QD M550, Maltrin® QD M600, and the modified corn starch Pure-Cote® B792, and the like.

Preferred starches are maltodextrins, in amounts ranging from about 0.01 to about 99%, preferably about 1 to about 70%, more preferably from about 2 to about 50% of the reagent formulations and even more preferably from about 5 to about 25% of the reagent formulations.

The surfactants used in the reagent formulations according to the present invention optionally comprise one or more selected from the group consisting of Atmos 300, Polysorbate 80, pluronic acid, sodium lauryl sulfate, mono and diglycerides of fatty acids, polyoxyethylene sorbitol esters, polyoxyethylene sorbitan fatty acid esters, .alpha.-hydro-.omega.-hydroxypoly(oxyethylene)poly(oxypropylene)poly(oxyethylene) block copolymers, polyoxyethylene alkyl ethers, polyoxyethylene castor oil derivatives, other like material, derivatives thereof, and various combinations thereof, without limitation.

Suitable surfactants include, but are not limited to, mono and diglycerides of fatty acids and polyoxyethylene sorbitol esters, such as, Atmos 300 and Polysorbate 80, and pluronic acid, sodium lauryl sulfate, and the like.

Preferred surfactants include mono and diglycerides of fatty acids and polyoxyethylene sorbitol esters, such as, Atmos 300 and Polysorbate 80 in amounts ranging from about 0.01 to about 20%, preferably about 0.05 to about 12%, more preferably from about 0.1 to about 7% of the reagent formulations and even more preferably from about 0.5 to about 5% of the reagent formulations.

In order to provide a palatable administration, it mar be desirable to add sweeteners to the reagent formulations. The sweeteners used in the reagent formulations according to the present invention can be selected from the group consisting of those well known in the art, including both natural and artificial sweeteners. A sweetener or combination of sweeteners may be included which is compatible with the biologically active agent and the other components such that a palatable system is produced. Sweeteners and flavors need not be added to reagent formulations intended for non-oral administration.

The sweeteners used in the reagent formulations according to the present invention optionally comprise one or more selected from the group consisting of aspartame (NutraSweet®), saccharine, saccharine salts, compressible sugars, fructose, sorbitol, mannitol, xylitol, L-sugars, maltose, sucrose, glucose, glycerin, dextrins, cyclamates, acesulfame K, thaumatin, sucralose, alitame, PS99/PS100, glycyrrhizin, monellin, stevioside, miraculin, other like material, derivatives thereof, and various combinations thereof, without limitation.

Maltodextrin and cyclodextran may also be added to provide palatable reagent formulations. Maltodextrin and cyclodextran are generally employed in order to dissipate unpleasant flavors within the reagent formulations, including for example and without limitation, the bitter taste of many biologically active agents. In addition, maltodextrin is a highly compressible powder which facilitates the formation of compressible sites of the present invention.

Suitable sweeteners include, but are not limited to, aspartame, acesulfame K, sucralose, sucrose, dextrose, maltodextrin, maltose, and the like.

Preferred sweeteners include aspartame, acesulfame K, sucralose, dextrose, maltodextrin, and maltose, in amounts ranging from about 0.01 to about 20%, preferably about 0.05 to about 115%, more preferably from about 0.1 to about 12% of the reagent formulations and even more preferably from about 0.5 to about 10% of the reagent formulations.

Further, suitable additives include thickeners, such as viscosity modifiers and thickening agents, for example sugars, polyvinylpyrrolidone, cellulosics, polymers, alginates, other like material, derivatives thereof, and various combinations thereof, without limitation.

Preferred reagent formulations, according to the present invention optionally comprise one or more triglycerides. Examples of triglycerides include vegetable oils such as corn oil, sunflower oil, peanut oil, olive oil, canola oil, soybean oil, other like material, derivatives thereof, and various combinations thereof, without limitation.

Added to the reagent formulation described above will be the appropriate biologically active agent or biologically active agents. As discussed, various types of biologically active agents are easily incorporated into the reagent formulations of the present invention.

It should be appreciated that there is considerable overlap between the aforementioned components in common usage, since a given component is often classified differently by different practitioners in the field, or is commonly used for any of several different functions. Thus, the aforementioned components should be taken as merely exemplary, and not limiting, of the types of components that can be included in the reagent formulations of the present invention. The amounts of such components can be readily determined by one skilled in the art, according to the particular properties desired.

(II) Tamper Resistant Pack

Preferably, the system of the present invention further comprises a tamper resistant pack.

Preferred tamper resistant packs, according to the present invention optionally comprise various types of tamper resistant packs, without limitation, provided they comprise tamper resistant packs that suitably secure the biologically compatible arrays of the present invention.

Any tamper resistant pack can be used, providing it is suitably compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

For example, one type of tamper resistant pack that may be used is the push to separate pack. Push to separate packs include lid materials of aluminum foil or an aluminum foil laminate. The base materials used in tamper resistant packs according to the present invention comprise one or more selected from the group consisting of single layer materials, laminates, and multi-layered materials, further comprising one or more layers selected from plastics such as PVC, polyamides, polyolefins, polyesters, and aluminum foil, other like material, variations thereof, and various combinations thereof, without limitation.

In an alternate embodiment, tamper resistant packs of the present invention feature a base which is covered by a lid foil. The lid may cover the entire base and is usually weakened in the region of each site. Each site may alternatively be covered with an individual breakaway lid element. A tab designed to align with the weakened region or lid, and for gripping enables separation of an individual site by pulling back the breakaway lid element, releasing the safety seal. The base, lid, and safety seal optionally comprise any of the above materials, without limitation.

The plastic lid, tabs, base, and safety seal of the present invention may be a plastic film or a plastic material made of the above mentioned materials. The plastic lid, tabs, base, and safety seal material for the push to separate pack are optionally an aluminum foil, laminates containing aluminum foil, or preferably a plastic with low elasticity and poor tensile strength, other like material, variations thereof, and various combinations thereof, without limitation.

Preferably, each site in the tamper resistant pack has a seal that is independently accessible, removable or breakable. Most preferably, the seal securely retains the biologically active agent(s) in a stable and sterile site until ready for use, at which time the site may be separated from the biologically compatible array.

Each site may have an independently removable or breakable seal. Once sealed, each site is moisture resistant, especially important in protecting agents manufactured by freeze drying or lyophilization. The independent seals allow the sealed surfaces of the sites to be individually bent and weakened so that the administration of one site may be easily separated and obtained without disturbing the sealed surfaces of adjacent sites. At the same time, the tamper resistant pack is sturdy enough to protect the administrations from physical stresses, especially those due to transportation and handling.

FIGS. 5A and 5B show the system of FIG. 1 secured in a tamper proof pack with a pull tab safety seal 84, 85 in accordance with one embodiment of the present invention.

Shown in FIG. 5A is a plan view of system 81 in which pull tab safety seal 87 is joined to the biologically compatible carrier at seams 84, 85. The pull tab 87 covers a plurality of adjacent sites 80, 71, 72, 73, contained in proximity. In the area of seams 84, 85, pull tab 87 is joined to biologically compatible carrier 82, for example, by sealing or adhesive bonding.

The system 81 is divided into an AM section 70 for morning administrations and a PM section 74 for evening administrations.

The AM section 70 of system 81 has tab 87 and two sites 80 and 71. Site 80 is an administration 83 made up of biologically active agents and site 71 is an administration 79 also made up of biologically active agents. The biologically active agents of administration 83 are incompatible with the biologically active agents of administration 79. Therefore, by securing administrations 83 and 79 in separate sites 80 and 71, respectively, the incompatible agents of administrations 83 and 79 are prevented from interacting with one another.

The PM section 74 of system 81 has tab 87 and two sites 72 and 73. Site 72 is an administration 78 made up of biologically active agents and site 73 is an administration 77 also made up of biologically active agents. The biologically active agents of administration 78 are incompatible with the biologically active agents of administration 77. Therefore, by storing administration 78 and administration 77 in separate sites 72 and 73, respectively, the incompatible agents of administration 78 and administration 77 are prevented from interacting with one another.

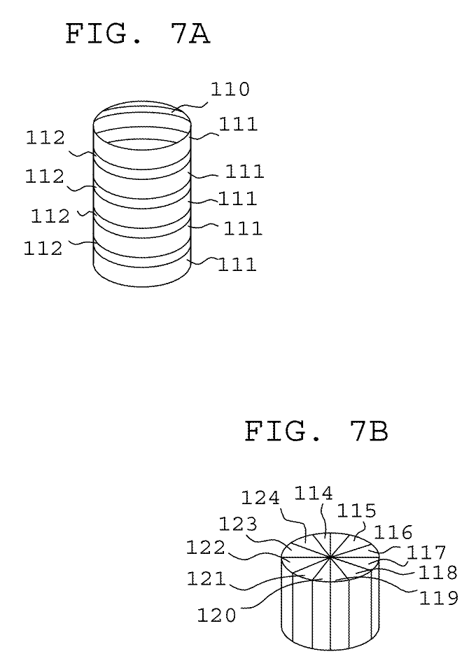

FIG. 5B shows the system 81 of FIG. 5A with a removable pull tab safety seal 87 partially removed and administration 83 separated 89 from the biologically compatible array, and the empty area 88 resulting from separation of administration. After removable seal 87 is removed, administrations 83, 79 can be separated. (note: FIG. 7B only depicts administration 83 as being separated 89).

FIGS. 6A and 6B show the system of FIG. 1 secured in a tamper proof pack with blister safety seals in accordance with one embodiment of the present invention.

Shown in FIG. 6A is a plan view of system 101 in which blister safety seal 107 is joined to the biologically compatible carrier at seams 104, 105. The blister safety seal 107 covers a plurality of adjacent sites 100, 91, 92, 93 contained in proximity. In the area of seams 104, 105, blister safety seal 107 is joined to biologically compatible carrier 102, for example, by sealing or adhesive bonding.

The system 101 is divided into an AM section 90 for morning administrations and a PM section 94 for evening administrations.

The AM section 90 of system 101 has blister safety seal 107 and two sites 100 and 91. Site 100 is an administration 103 made up of biologically active agents and site 91 is an administration 99 also made up of biologically active agents. The biologically active agents of administration 103 are incompatible with the biologically active agents of administration 99. Therefore, by securing administrations 103 and 99 in separate sites 100 and 91, respectively, the incompatible agents of administrations 103 and 99 are prevented from interacting with one another.

The PM section 94 of system 101 has a blister safety seal 107 and two sites 92 and 93. Site 92 is an administration 98 made up of biologically active agents and site 93 is an administration 97 also made up of biologically active agents. The biologically active agents of administration 98 are incompatible with the biologically active agents of administration 97. Therefore, by storing administration 98 and administration 97 in separate sites 92 and 93, respectively, the incompatible agents of administration 98 and administration 97 are prevented from interacting with one another.

FIG. 6B shows system 81 of FIG. 5A with blister safety seal 107 partially removed and administration 103 separated 109 from the biologically compatible array, and empty area 108 resulting from separation of administration. After blister safety seal 107 is punctured, administrations 103, 109 can be separated. (note: FIG. 7B only depicts administration 103 as being separated 109).

Preferably, groups of sites in the tamper resistant pack include surrounding perforations, allowing groups of sites to be separated from the whole tamper resistant pack.

Another embodiment of the present invention comprises providing tamper resistant packs further comprising suitably compatible control codes corresponding to the administrations. Preferably, the tamper resistant pack comprises suitably compatible control codes to indicate which biologically active agents are to be emplaced and where the biologically active agents are to be emplaced.

Preferably, the tamper resistant pack comprises suitably compatible control codes that comprise at least one graphic image per site.

Preferably, the tamper resistant pack comprises suitably compatible control codes that further comprise at least one graphic image per site that further comprise barcodes.

Preferably, the tamper resistant pack comprises suitably compatible barcodes that are machine readable, by barcode scanners or the equivalent.

Additionally, the suitably compatible control codes optionally provide a reliable and effective production control system in that the user can effectively emplace the proper biologically active agents at each site on the biologically compatible array by comparing the biologically compatible control codes at each site with a master record. The user can prevent production errors and thus assure that the correct biologically active agents are emplaced at each site through the use of barcode scanning devices which scan the suitably compatible control codes during the production methodology.

The method of the present invention thus comprises, in an alternate embodiment, devices which scan the suitably compatible control codes during the production methodology. Such devices may include barcode scanners, without limitation. It is well known in the art that improved levels of production errors and improved production monitoring are thereby realized.

Further, the suitably compatible control codes optionally provide a reliable and effective feedback system in that the user can determine if the proper administrations have been taken on the proper days and at the proper times by comparing the biologically compatible control codes with a master record. The user or monitoree can effectively monitor and determine if an administration has been missed, prevent improper administrations, or administrations at improper times.

Preferably, the tamper resistant pack comprises suitably compatible control codes that can effectively monitor and verify whether a user has administered the correct biologically active agents.

Preferably, the tamper resistant pack comprises suitably compatible control codes that can effectively monitor and verify whether a user has administered the correct biologically active agents at the correct time.

The application of the system of the present invention is further specified in the application section below.

Another embodiment of the present invention comprises providing tamper resistant packs further comprising suitably compatible user instructions corresponding to the administrations. Preferably, the tamper resistant pack includes suitably compatible user instructions to give an indication as to what administration is required to be taken and when.

Additionally, the suitably compatible user instructions provide a feedback system in that the user can determine and confirm proper administration by comparing the suitably compatible user instructions with a calendar or clock. This helps assure administration is performed on the proper days and at the proper times. The user has a means that provides a degree of feedback in determining if an administration has been missed, an improper administration taken, or an administration taken at an improper time.

It is noted that any user dependent feedback system relying solely on user instruction as described herein or otherwise disclosed, is limited to the level of skill of the user and it thus subject to errors and misuse, caused by factors referenced above. (See Libow et al., Parkin et al., Seidl et al., and Schwartz et al.)

Preferably, at least the top of at least one site is transparent or translucent and the site color is used as the suitably compatible user instruction.

Preferably the site color is used to indicate when to take the administration although it will be appreciated by those skilled in the art that other suitably compatible user instructions may also apply such as different administrations or biologically active agents.

Preferably, the tamper resistant pack, or combinations thereof, as a whole or in part, such as a site or sites, row or rows, column or columns, are colored. Preferably the color coding is part of a logical progression such as a rainbow color spectrum, traffic light range, or other cultural familiar range of colors to show an appropriate order of use or consumption.

Preferably the color coding comprises the biologically compatible array, the reagent formulation, the tamper resistant pack, or combinations thereof, without limitation.

Preferably, the range of colors comprises the biologically compatible array, the reagent formulation, or the tamper resistant pack, or combinations thereof, and further comprises a recognizable sequence.

Preferably, suitably compatible user instructions comprise a key defining or explaining the color coding. For example, morning sites are colored yellow corresponding to the rising sun, and evening sites are colored orange corresponding to the setting sun, or other colors and combinations thereof, without limitation.

Preferably, a color key is provided on the tamper resistant pack to indicate which color corresponds with which administration date or time. The color key comprises suitably compatible user instructions and may be provided on the biologically compatible array, directly on the sites, the tamper resistant pack, and combinations thereof, without limitation.

Preferably, time suitably compatible user instructions may be incorporated into the tamper resistant pack of the present invention. The time suitably compatible user instructions may be of any type, without limitation. The time suitably compatible user instructions correspond to at least two distinct time periods, but may correspond to any number of distinct time periods without limitation. For example, without limitation, the time suitably compatible user instructions may indicate a general time of the day corresponding to each of the sites or a specific time of the day corresponding to each of the sites. Time suitably compatible user instructions may be indicated directly on the sites, another portion of the biologically compatible array, the tamper resistant pack, and combinations thereof, without limitation.

Preferably, time suitably compatible user instructions include markings selected from the group consisting of: AM, PM, morning, day, daytime, afternoon, evening, night, nighttime, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 37, 38, 39, 40, 41, 42, 43, 44, 45, 45, 46, 47, 48, 49, 50, 51, 52, 53, 54, 55, 56, 57, 58, 59, 60, One, Two, Three, Four, Five, Six, Seven, Eight, Nine, Ten, Eleven, Twelve, Thirteen, Fourteen, Fifteen, Sixteen, Seventeen, Eighteen, Nineteen, Twenty, Twenty One, Twenty Two, Twenty Three, Twenty Four, Twenty Five, Twenty Six, Twenty Seven, Twenty Eight, Twenty Nine, Thirty, Thirty One, Thirty Two, Thirty Three, Thirty Four, Thirty Five, Thirty Six, Thirty Seven, Thirty Eight, Thirty Nine, Forty, Forty One, Forty Two, Forty Three, Forty Four, Forty Five, Forty Six, Forty Seven, Forty Eight, Forty Nine, Fifty, Fifty One, Fifty Two, Fifty Three, Fifty Four, Fifty Five, Fifty Six, Fifty Seven, Fifty Eight, Fifty Nine, Sixty, and the like, and combinations thereof, without limitation.

Preferably, day suitably compatible user instructions are incorporated into the tamper resistant pack of the present invention. The day suitably compatible user instructions may be of various types, without limitation. The day suitably compatible user instructions correspond to at least two distinct sites. For example, without limitation, the day suitably compatible user instructions may be a specific day of the week, or an abbreviation of said day, a specific date, or a general succession of days. Day suitably compatible user instructions may be indicated directly on the sites, another portion of the biologically compatible array, the tamper resistant pack, and combinations thereof, without limitation.

Preferably, day suitably compatible user instructions include markings selected from the group consisting of: Monday, Tuesday, Wednesday, Thursday, Friday, Saturday, Sunday, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and the like, and combinations thereof, without limitation.

Preferably, week suitably compatible user instructions are incorporated into the tamper resistant pack of the present invention. The week suitably compatible user instructions may be of various types, without limitation. The week suitably compatible user instructions correspond to at least two distinct sites. For example, without limitation, the week suitably compatible user instructions may be a week of the month, or an abbreviation of said week, a specific date, or a general succession of weeks. Week suitably compatible user instructions may be indicated directly on the sites, another portion of the biologically compatible array, the tamper resistant pack, and combinations thereof, without limitation.

Preferably, week suitably compatible user instructions include markings selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and the like, and combinations thereof, without limitation.

Preferably, month suitably compatible user instructions are incorporated into the tamper resistant pack of the present invention. The month suitably compatible user instructions may be of various types, without limitation. The month suitably compatible user instructions correspond to at least two distinct sites. For example, without limitation, the month suitably compatible user instructions may be a month of the year, or an abbreviation of said month, a specific date, or a general succession of months. Month suitably compatible user instructions may be indicated directly on the sites, another portion of the biologically compatible array, the tamper resistant pack, and combinations thereof, without limitation.

Preferably, month suitably compatible user instructions include markings selected from the group consisting of: J, F, M, A, M, J, J, A, S, O, N, D, Jan, Feb, Mar, Apr, May, Jun, Jul, Aug, Sep, Oct, Nov Dec, January, February, March, April, May, June, July, August, September, October, November, December, and combinations thereof, without limitation.

Preferably, quarter suitably compatible user instructions are incorporated into the tamper resistant pack of the present invention. The quarter suitably compatible user instructions may be of various types, without limitation. The quarter suitably compatible user instructions correspond to at least two distinct sites. For example, without limitation, the quarter suitably compatible user instructions may be a quarter of the year, or an abbreviation of said quarter, a specific date, or a general succession of quarters. Quarter suitably compatible user instructions may be indicated directly on the sites, another portion of the biologically compatible array, the tamper resistant pack, and combinations thereof, without limitation.

Preferably, quarter suitably compatible user instructions include markings selected from the group consisting of: 1,2,3,4, JFM, AMJ, JAS, OND, and combinations thereof, without limitation.

Suitably compatible user instructions can include the time, for example ‘AM’ or ‘PM’, the day, for example ‘Monday’ or ‘Tuesday’, the week number, for example ‘1’ or ‘2’, the month for example ‘January’ or ‘February’, or the quarter, for example ‘JFM’ or ‘AMJ’, or any other identifier, and combinations thereof, without limitation.

Preferably, suitably compatible user instructions comprise color, time, day, week, month, quarter, for each separate row or column, each site, each type of biologically active agent, each time, each day, each week, each month, each quarter, and combinations thereof, without limitation.

Tamper resistant packs suitable for the present invention can be configured in a sheet formation, rolled formation, multidimensional formation, or any other formation, or combination of formations, without limitation.

In one embodiment, the tamper resistant pack is configured in a planar formation such that the sites form a series of rows and columns.

In an alternative embodiment, the tamper resistant pack is configured in a rolled formation such that as a strip of sites is pulled, the administration to be taken next is revealed from the container.

In other alternative embodiments, the tamper resistant pack of the present invention can be configured in a sheet formation, rolled formation, multidimensional formation, or any other formation, or combination of formations, without limitation.

Thus, the system of the present invention is not strictly limited to geometries that are planar. Any efficacious geometry is suitable, provided the tamper resistant pack may be produced using biologically compatible components. Non-limiting exemplary geometries include bottles, canisters, packets, tubes, vials, and the like.

It should be appreciated that there is considerable overlap between the aforementioned components in common usage, since a given component is often classified differently by different practitioners in the field, or is commonly used for any of several different functions. Thus, the above listed components should be taken as merely exemplary, and not limiting, of the types of components that can be included in biologically compatible carriers of the present invention. The amounts of such components can be readily determined by one skilled in the art, according to the particular properties desired.

(III) Outer Packaging

Preferably, the system of the present invention further comprises outer packaging.

Preferred outer packaging, according to the present invention optionally comprises various types of outer packaging, without limitation.

Any outer packaging can be used that is suitably compatible, providing it is compatible with the (I) the biologically compatible array comprising (i) the biologically compatible carrier, (ii) the biologically compatible control codes, (iii) the biologically compatible user instructions, (iv) a plurality of sequenced sites of varying isolated formulations of biologically active agents, (v) the reagent formulations supporting effective delivery of and admixed with the biologically active agents, and (II) the tamper resistant pack, (III) the outer packaging, (IV) the applications to perform administrations, and (V) the methods for producing the system of the present invention.

In one embodiment, the system includes a container for receiving and suitably storing the composition of the present invention. Preferably, the container is divided into a plurality of discrete compartments, wherein at least one compartment is for receiving and suitably storing one or more selected from the group consisting of a biologically compatible array, a tamper resistant pack, and a loose, unpackaged, or variably packaged biologically active agent and at least one compartment is for receiving and suitably storing one or more selected from the group consisting of a biologically compatible array, a tamper resistant pack, and a loose, unpackaged, or variably packaged biologically active agent, along with printed material and/or audio/visual aids to assist in compliance with a meticulous administration plan.

FIG. 10 shows a perspective view of a storage container 160 with several cavities 162 formed by dividers 161 for long term storage of a multitude of tamper resistant packs as depicted in FIG. 5A and 6A. The storage container 160 also incorporates suitably compatible user instructions within the storage container or on the exterior of the container 166, which identify the contents. Additional suitably compatible user instructions include lot number 165, expiration date 164, and barcode 163. The contents are secured within storage container 160 by folding and securing flaps 167.

In one embodiment, the outer packaging is configured to accommodate the present invention in a planar formation such that the present invention in a planar formation is suitably stored.

In an alternative embodiment, the outer packaging is configured to accommodate the present invention in a in a rolled formation such that the present invention in a rolled formation is suitably stored.

In other alternative embodiments, the outer packaging of the present invention can be configured such that the present invention in a sheet formation, rolled formation, multidimensional formation, or any other formation, or combination of formations, without limitation, is suitably stored.

Thus, the system of the present invention is not strictly limited to geometries that are planar. Any efficacious geometry is suitable, provided the biologically compatible array may be produced using biologically compatible components. Non-limiting exemplary geometries include bottles, canisters, packets, tubes, vials, and the like.

(IV) Applications to Perform Administrations

Preferably, the system of the present invention further comprises applications to perform administrations of the present invention.

Preferred applications according to the present invention comprise (a) the user administering varying biologically active agents(s) from the biologically compatible array into, onto, or near the target bodily cavity or area of an animal, (b) allowing the biologically active agents(s) to dissolve within, on, or near the target bodily cavity or area; and (c) releasing the biologically active agents into, onto, or near the target bodily cavity or area.

The application of the present invention is the use of a sequenced biologically compatible delivery system to provide tailored delivery of biologically active agents by a user to an animal and increase the ease and precision with which a meticulous administration plan is delivered by a user to an animal.

Preferably, the animal is a human. This should not be seen as limiting, however, as users administering a treatment plan to an animal, for example a pet, may also benefit from the system of the present invention. For example, a course of anthelmintic treatments for treatment of nematode worms in an animal the application of which is accomplished by the user who is an owner or caregiver to the animal.

The user can be an association, company, organization, practitioner, patient, human, or other animal, preferably a mammal, and more preferably a human.

The application of the present invention may be used by any human or other animal. The present system, application, and method are particularly suitable for individuals with special therapeutic requirements or specific therapeutic requirements, particularly where those requirements would benefit from a meticulous administration plan.

For example, without limitation, the application of the present invention is particularly suitable for children, seniors, individuals suffering from infirmity, the chronically ill, menopausal women, lactating women, pregnant women, men or women planning to conceive a child, individuals suffering from a pathological condition, individuals who require convenient feedback or monitoring, or any combination of the above.

It may be desirable to include additives discussed above to improve the application of the present invention. Exemplary additives included buffering agents, and release agents, without limitation.

A buffering agent may be desirable to place the administration in a favorable pH environment in the target bodily cavity or area. For example, passage across the mucosal tissues of the mouth, pharynx, and esophagus, without limitation.

The rate of dissolution of the administration within the user's bodily cavity may be controlled chemically, as well as physically, through the extent of compression of the reagent formulations. Releasing agents may enhance dissolution or they may inhibit dissolution as necessary. As will be appreciated by those skilled in the art, however, modulating the particle size of the components in the reagent formulation and/or the density can provide a similar effect, providing improved manufacturability, optimization of erosion and dissolution rates, without addition of a releasing agents.

In an alternative embodiment the application of the present invention may include reagent formulation additives, well known to those of ordinary skill, which aid dissolution in co-administered carrier fluid for contact, consumption, exposure, or insertion.

In preferred embodiments, the administration applied/used taken is one site per day.

In preferred embodiments, the administration applied/used taken is two sites per day.

In preferred embodiments, the administration applied/used taken is a multiplicity of sites per day.

The application of the present inventions may also comprise providing an additional administration containing a biologically active agent in a site in the biologically compatible array such that the additional administration is located adjacent to the first administration.

In preferred embodiments, the administration within each site contains an uneven administration and in one embodiment, each site contains an administration that includes two or more different varieties of concentration, type or volume of biologically active agent for each administration.

Preferably, the administration to be taken is varied depending on the biologically active agent to be taken, the concentration and/or volume of biologically active agent, and/or the time of day, week, month, quarter.

FIG. 1 shows a plan view of a novel sequenced biologically compatible delivery system in accordance with one embodiment of the present invention. The system shown in FIG. 1 generally indicated by arrow 16 contains one row of sites 17. Each site 21 contains a specific concentration and ratio of biologically active agents (‘the administration’). For example, each individual site 10 is an administration 21. In this embodiment, the system 16 contains four sites containing four administrations 10, 12, 13, and 14. The system 16 is divided into an AM section for morning administrations and a PM section for evening administrations. For a complete 1 day course of administrations 10, 12, 13, and 14, one system 17 is used.

Administrations 10, 12, 13, and 14 contain incompatible biologically active agents. For example, administration 10 contains biologically active agents which are incompatible with biologically active agents in administration 12. Thus, within the row, the administrations in the four sites each contain incompatible biologically active agents. Perforations 18 are included around sites 21 on the system 16 to allow the user to detach administrations 10, 12, 13, and 14. The system may further be incorporated into a tamper resistant pack shown in FIGS. 5A-5B. In addition, system 16 may further be incorporated into an outer packing shown in FIG. 10. The biologically compatible array, tamper resistant pack and outer packing may have suitably compatible user instructions indicating to the user as to when to use each of the administrations 10, 12, 13, and 14.

FIGS. 5A and 5B show the system of FIG. 1 secured in a tamper proof pack with a pull tab safety seal 84, 85 in accordance with one embodiment of the present invention.

Shown in FIG. 5A is a plan view of system 81 in which pull tab safety seal 87 is joined to the biologically compatible carrier at seams 84, 85. The pull tab 87 covers a plurality of adjacent sites 80, 71, 72, 73, contained in proximity. In the area of seams 84, 85, pull tab 87 is joined to biologically compatible carrier 82, for example, by sealing or adhesive bonding.

The system 81 is divided into an AM section 70 for morning administrations and a PM section 74 for evening administrations.

The AM section 70 of system 81 has tab 87 and two sites 80 and 71. Site 80 is an administration 83 made up of biologically active agents and site 71 is an administration 79 also made up of biologically active agents. The biologically active agents of administration 83 are incompatible with the biologically active agents of administration 79. Therefore, by securing administrations 83 and 79 in separate sites 80 and 71, respectively, the incompatible agents of administrations 83 and 79 are prevented from interacting with-one another.

The PM section 74 of system 81 has tab 87 and two sites 72 and 73. Site 72 is an administration 78 made up of biologically active agents and site 73 is an administration 77 also made up of biologically active agents. The biologically active agents of administration 78 are incompatible with the biologically active agents of administration 77. Therefore, by storing administration 78 and administration 77 in separate sites 72 and 73, respectively, the incompatible agents of administration 78 and administration 77 are prevented from interacting with one another.

In one embodiment of the present invention, the key delivery step using the present application is separating the administration 80, 71, 72, 73, shown in FIG. 5A from the tamper resistant pack by rupturing seal 87 by hand, exposing the administration 80, 71, 72, 73, within seal 87, and separating and applying the administration 80, 71, 72, 73, into, onto, or near the target bodily cavity or area.

FIG. 5B shows the system 81 of FIG. 5A with a removable pull tab safety seal 87 partially removed and administration 83 separated 89 from the biologically compatible array, and the empty area 88 resulting from separation of administration. After removable seal 87 is removed, administrations 83, 79 can be separated. (note: FIG. 7B only depicts administration 83 as being separated 89).

The application of the present invention comprises, in one embodiment, biologically compatible user instructions which provide a feedback system in that the user can determine and confirm proper administration by comparing the biologically compatible user instructions with a calendar or clock. This helps assure administration is performed on the proper days and at the proper times. The user has a means that provides a degree of feedback in determining if an administration has been missed, an improper administration taken, or an administration taken at an improper time.

It is noted that any user dependent feedback system relying solely on user instruction described herein or otherwise disclosed, is limited to the level of skill of the user and it thus subject to errors and misuse, caused by factors referenced above. (See Libow et al., Parkin et al., Seidl et al., and Schwartz et al.)

The application of the present invention comprises, in another embodiment, devices which scan the biologically compatible control codes during the administration application. Such devices may include barcode scanners, without limitation. It is well known in the art that significantly improved levels of user errors and significantly improved user monitoring are thereby realized.

Further, the biologically compatible control codes optionally provide a reliable and effective feedback system in that the user can determine if the proper administrations have been taken on the proper days and at the proper times by comparing the biologically compatible control codes with a master record through the use of the aforementioned devices which scan the biologically compatible control codes when the user dispenses the site during the administration application.

The user can effectively monitor and determine if an administration has been missed, prevent improper administrations, or administrations at improper times. Errors and/or misuse can also be reported to suitable monitorees by communications devices well known to those of ordinary skill in the art. Most critically, intervention by suitable means and by monitorees is available whenever errors and/or misuse are reported. This has the effect of preventing, or at least correcting errors and misuse which otherwise go undiscovered.

The application of the present invention comprises any use of the system of the invention for providing improved therapeutic care to an animal by increasing compliance with a meticulous administration plan and facilitating sequential administration of incompatible agents in a convenient, cost-friendly, simple and effective manner.

Preferably, the application of the present invention includes an administration plan. More preferably, the administration plan is a daily administration plan. Even more preferably, the daily administration plan is a meticulous daily administration plan.

The application of the present invention comprises administering incompatible agents and/or prescription and/or non-prescription biologically active agents together from the biologically compatible array to an animal at the time indicated by the biologically compatible user instructions, which enables compliance with a meticulous daily administration plan, optionally monitored by the biologically compatible control codes, and optionally comprising reporting of errors and/or misuse of the system to suitable monitorees, and intervention therefor by suitable means to assure compliance.

(V) Methods for Producing the System of the Present Invention

A method for producing the system of the present invention begins with preparation of the biologically compatible carrier formulation and the reagent formulations, along with any preparation of the biologically active agents.

Preferred biologically compatible carriers according to the present invention optionally comprise a base solution that includes at least one component from each of the groups comprising: water soluble polymers, water, and fillers. Typically, the base solution is prepared by adding an initial mixture of dry or wet components to water that is stirred.

To the base solution, additional components are added, such as antimicrobial agents, binding agents, coloring agents, cooling agents, emulsifying agents, fillers, flavoring agents, glycols, humectants, plasticizers, preservatives, release agents, saliva stimulating agents, stabilizers, starches, surfactants, sweeteners, water soluble polymers, water, other like material, derivatives thereof, and various combinations thereof, without limitation.

The plasticizers used in the biologically compatible carriers are preferably previously dissolved or dispersed in a solvent and added in solution form, along with surfactants, emulsifying agents, other additives, and any of the biologically compatible carrier components these agents act upon. This improves the function of these agents and therefor the properties biologically compatible carriers of the present invention.

Any of the aforementioned biologically compatible carriers of the present invention may be mixed, coated onto a suitable transport material and dried. The biologically compatible carrier in solution form must have a surface tension which allows even dispersion across the intended width of, and post drying release from, the transport material.

The coating of the biologically compatible carrier in solution form onto the suitable transport material can be performed using any means. Suitable transport materials include non-siliconized polyethylene terephthalate film, non-siliconized kraft paper, polyethylene-impregnated kraft paper, or non-siliconized polyethylene film. The formulation of the biologically compatible carrier of the present invention is typically cast on such suitable transport material and dried to form the biologically compatible carrier.

For example, and without limitation, conventional coating equipment may be used. A more preferred coating technique is a knife-over-roll coating head. The thickness of the resulting biologically compatible carrier of the present invention depends on the concentration of solids and on the gap of the coating head and can vary between 5 and 200 .mu.m.

The biologically compatible carrier is then preferably air-dried or dried under warm air, in an elevated temperature air-bath using a drying oven, drying tunnel, vacuum drier, or any other suitable drying equipment, which does not adversely affect the components or palatability of the carrier.

The dried biologically compatible carrier can contain from about 0.1% to about 10% moisture, preferably from about 3% to about 8% moisture, even more preferably from about 4 to about 7% moisture.

It is noted that in other embodiments, preferred biologically compatible carriers can be produced by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

It should be appreciated that any suitable type, number and organization of process procedures or steps (i.e., mixing, heating, drying, cooling, addition of components, printing, dot dispensing, extruding, molding, milling, spraying, sifting, screening, atomization, static agitation, mechanical agitation, coating), process parameters (i.e., temperature, pressure, pH, process times) or the like can be utilized.

The biologically compatible carrier is then transported by such suitable transport materials disclosed above, or an equivalent effective means, into the station, or stations, wherein the biologically compatible control codes and biologically compatible user instructions are applied.

Further, it is noted that in other embodiments, preferred biologically compatible control codes and biologically compatible user instructions according to the present invention can then be applied to the biologically compatible carrier by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

As discussed above, preferably the system of the present invention further comprises biologically compatible control codes applied to the biologically compatible array corresponding to the sites on the biologically compatible arrays that further comprise at least one graphic image per site, that still further comprise barcodes that are machine readable by barcode scanners or the equivalent.

Preferably, the biologically compatible control codes indicate which biologically active agents are to be emplaced on the biologically compatible carrier.

Preferably, the biologically compatible control codes indicate where the biologically active agents are to be emplaced on the biologically compatible carrier.

The method of producing the present invention comprises, in another embodiment, devices which scan the biologically or suitably compatible control codes during the production methodology. Such devices may include barcode scanners, without limitation. It is well known in the art that significantly improved levels of production errors and significantly improved production monitoring are thereby realized.

Thus, the method of producing the present invention optionally comprises, in another embodiment, a reliable and effective production control system in that the user can effectively emplace the proper biologically active agents at each site on the biologically compatible array by comparing the biologically or suitably compatible control codes at each site with a master record, through the use of suitably machine readable control codes and suitable machine readers. The user can prevent production errors and thus assure that the correct biologically active agents are emplaced at each site on the biologically compatible array through the use of, for example, barcode scanning devices which scan the biologically or suitably compatible control codes during the production methodology, without limitation.

It should again be appreciated that any suitable type, number and organization of process procedures or steps (i.e., mixing, heating, drying, cooling, addition of components, printing, dot dispensing, extruding, molding, milling, spraying, sifting, screening, atomization, static agitation, mechanical agitation, coating), process parameters (i.e., temperature, pressure, pH, process times) or the like can be utilized.

The biologically compatible carrier is then transported by such suitable transport materials disclosed above, or an equivalent effective means, into the station, or stations, wherein the reagent formulations are applied and secured to the biologically compatible carrier.

After each of the reagent formulation components is mixed with the other reagent formulation components, and the biologically active agents, they may be applied and secured to the biologically compatible carrier, in two or more sites, to create the biologically compatible array.

With many biologically active agents, it has previously been difficult to provide a palatable administration because of the bitterness or other unpleasant taste of many biologically active agents. According to the present invention, improved taste characteristics are presented by adding various flavors, sweeteners, and the like. Maltodextrin and cyclodextran may be added to provide palatable reagent formulations, without limitation. Maltodextrin and cyclodextran may generally be employed in order to dissipate unpleasant flavors within the reagent formulations, including for example and without limitation, the bitter taste of many biologically active agents. In addition, maltodextrin is a highly compressible powder which facilitates the formation of compressible sites of the present invention, when compression is suitable. When the components are combined in compressible administrations according to the present invention, as solids, liquids, semi-solids, semi-liquids or combinations thereof, problems associated with combining insoluble reagent formulation components are avoided. Other well known flavorings, in addition to those described herein, may also be acceptable because of the ease of processing the components of the present invention.

The plasticizers used in the reagent formulations are preferably previously dissolved or dispersed in a solvent and added in solution form, along with surfactants, emulsifying agents, other additives, and any of the reagent formulation components these agents act upon. This improves the function of these agents and therefor the properties sequenced sites of the present invention.

As discussed herein the rate of dissolution of the administration within the user's bodily cavity may be controlled, in part, by including a release agent in the reagent formulations. The rate of dissolution of the administration within the user's bodily cavity may also be controlled chemically, as well as physically, through the extent of compression of the reagent formulations.

In addition releasing agents may further be included to facilitate the processes of producing the sites, such as mold release, or strip release. Releasing agents are also useful in those embodiments wherein a powder mixture is funneled into a chute during manufacture. Releasing agents, and also surfactants, improve product flow and avoid static electricity charge buildup within the reagent formulation which may cause the components to separate due to electrostatic forces.

The solubilizers discussed herein also may improve processing. The amount of solubilizer that can be included in reagent formulations of the present invention is not particularly limited. Of course, in reagent formulations that are administered to a user, the amount of a given solubilizer is limited to a biologically acceptable amount, which is readily determined by one of skill in the art. In some circumstances, it may be advantageous to include amounts of solubilizers far in excess of biologically acceptable amounts, for example, to maximize the concentration of the biologically active agent, with excess solubilizer removed prior to providing the administration to a user using conventional techniques, such as distillation or evaporation.

The aforementioned components described above and otherwise discussed herein are commonly utilized to facilitate the processes involving the preparation of the reagent formulation, the encapsulation coating, or the administration.

It is noted that in other embodiments, preferred reagent formulations according to the present invention can be formed and secured to the biologically compatible carrier by at least one method selected from the group consisting of agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes, well known to those of ordinary skill in the art.

The present invention discloses methods of producing sequenced sites of administrations containing one or more varying biologically active agents organized on a common biologically compatible carrier. One embodiment of the present invention overcomes many of the problems encountered generally in incorporating biologically active agents into a reagent formulation. For example, the present invention discloses the mixing of solid powders or liquids at room temperature, when compressible administration are desirable, for example and without limitation, as opposed to liquid components at elevated temperatures, preventing the degradation of biologically active agents, which often occurs at elevated temperatures. This facilitates use of biologically active agents having relatively low melting points, or those biologically active agents which can experience decomposition below their melting points. The mixing and other processes can be done at very low temperatures, if such is desirable.

Further, evaporation of any volatile components is minimized and the use of dry components improves flow, enhancing processing.

Further, because solid powders or liquids are combined together at low temperatures, components which may be chemically inappropriate when in a heated solution or suspension can be mixed.

In addition, biologically active agents, flavorings, and other components which are insoluble when placed in the same liquid environment may be processed as part of a compressible reagent formulation.

Each of the components is mixed with the other components to produce the reagent formulations of the present invention. It is presently preferred to use the method of repetitive dilution in mixing the various components. Using this method, first the two smallest components by weight, as a proportion of the final administration, are mixed together thoroughly.

After these two components are completely mixed, the next smallest component or components are added and mixed thoroughly. This procedure is repeated until all of the components are added and mixed thoroughly.

After the desired components are thoroughly mixed, they may be formed into a solid site, or sites wherein compression is desirable, without limitation. In other cases the components are wetted to form a slurry, dried, and then compressed to form the site.

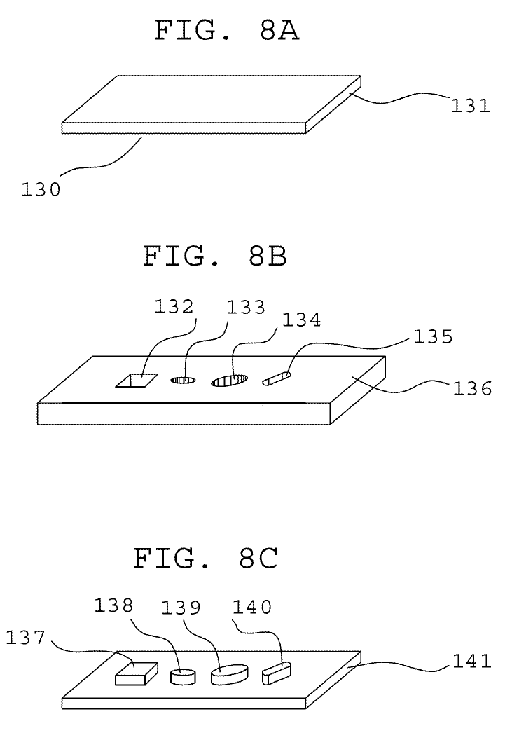

For example, forming the sites may be by compression, without limitation, and using molds, without limitation. FIGS. 8A-8C show perspective views of one embodiment of a mold assembly that can be used for molding the reagent formulation components of the present invention. The mold assembly may comprise three separate components. For example, die 131 shown in FIG. 8A, mold cavity 136 shown in FIG. 8B and raml4l shown in FIG. 8C. Each component of the mold assembly can be separated in order to facilitate release of the sites once they are formed.

The interior of the mold 136 shown in FIG. 8A includes cavities 132,133,134,135 formed in any desired shape so that the reagent formulation components described above can be compressed or molded to form appropriately shaped sites.

Ram 141 shown in FIG. 8C fits into the mold cavity 136 shown in FIG. 8B and compresses the reagent formulation components into mold cavities 132, 133, 134, 135 to form the sites. Ram 141 shown in FIG. 8C may have recesses in dies 137, 138, 139, 140 that accommodate a safety seal. Thus, the safety seal can be placed over the site. Ram 141 then compresses the sites tightly under the safety seal. Following compression of the site, the safety seal is securely pressed in place. The safety seal may take various shapes to accommodate sites of various shapes, as further discussed below.

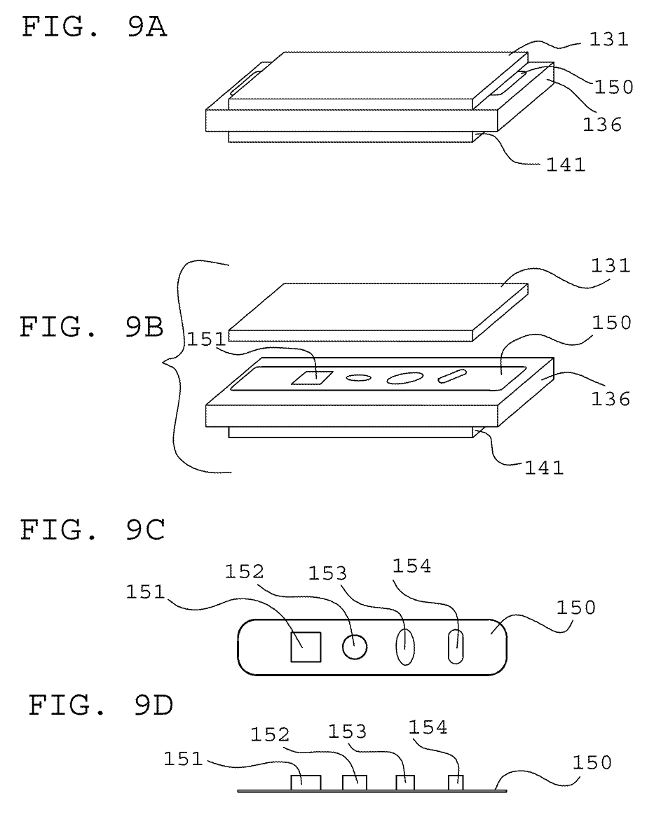

FIGS. 9A-9B show perspective views of the mold assembly of FIG. 8A-8C molding a system of the present invention. The resulting system of the present invention 150 is shown in FIGS. 9C-9D.

As discussed above, the mold assembly may comprise three separate components. For example, die 131, mold cavity 136, and ram 141 shown assembled in FIG. 9A. Each component of the mold assembly can be separated in order to facilitate release of the sites once they are formed. The mold assembly shown in FIG. 9A molds system 150.

The interior of the mold cavity 136 shown in FIG. 9B includes cavities 132,133,134,135 as shown in FIG. 8B, formed in any desired shape so that the reagent formulation components described above can be compressed or molded to form appropriately shaped sites.

In the embodiment of the present invention shown in FIG. 9B, the mold assembly for forming sites of the present invention may have die cavities 132, 133, 134, 135 as shown in FIG. 8B. The mold assembly has die components configured to form the sites. The die components may have concave surfaces. To prepare sites using the mold assembly, a quantity of reagent formulation is placed in the die 131 on the concave surface. A biologically compatible carrier 150 is positioned such that a portion is within the die 131. Additional amounts of reagent formulations are, or alternatively, were previously placed on the appropriate sites on the biologically compatible carrier, which is now suitably aligned and resting on the ram 141. The die 131 and ram 141 then compress the reagent formulation on the biologically compatible carrier thereby preparing the administrations and forming the sites. Then, in order to remove the administrations from the mold, the die 131 pushes the biologically compatible array out of the mold cavity 136. As a result, the compressed powder reagent formulation is held together by physical means rather than by chemical means.

FIG. 9B shows the interior of the mold cavity 136 holding a completed system 150 after molding.

The resulting molded system of the present invention 150 is shown in FIGS. 9C-9D after removal from the mold assembly with molded sites 151, 152, 153, 154.

An additional embodiment of the administration of the present invention shown in FIG. 7A has alternating layers 110, 111 of reagent formulation containing biologically active agents.

Each alternating layer 110, 111 is shaped with dimensions varied according to particular needs. The number of layers and the formulation of layers 110, 111 can be easily varied to meet particular user needs. Various concentrations of a biologically active agent, or even multiple biologically active agents, may be administered in this manner. This greatly simplifies inclusion of incompatible formulations, where such are required to be administered together. Thus, the method of production of the biologically compatible array can be adapted to produce various administrations to fit varying circumstances.

In a further embodiments of an administration, of the present invention, the reagent formulation is divided laterally along the site, pie-shaped segments of reagent formulation are pressed together, or reagent formulation segments may alternate axially around the periphery of the site as shown in FIG. 7B, with segments 114-124. Alternatively, the dimensions of the segments or layers may be varied to provide other appropriate amounts of biologically active agent.

In certain alternative embodiments, the reagent formulation is compressed under relatively high forces to form an administration. Compressive forces in the range of from approximately 500 psi to approximately 2000 psi are presently preferred however, any force which is sufficient to compress the components into a coherent, integrated administration could be used.

For administrations that so require, the extent of the compressive forces can be modified to vary the rate that the administration will dissolve in a user's mouth. The greater the compressive forces that form the mixture, the slower the dissolution of the reagent formulation in the mouth.

When employing this method to form the compressible sites of the present invention, for example, there is no need to heat the mixture to a molten state as has been the practice in the past in forming some biologically active agent containing administrations. As a result, heat degradation of the biologically active agent component is avoided while proper mixing and a uniform product are provided.

In other embodiments, according to the present invention, the desired components are formed into the site and secured to the biologically compatible carrier by printing, dot dispensing, dehydration, freeze drying or lyophilization, pouring into a mold, spraying onto a suitable holder, vapor deposition, or other known techniques in the art, for example and without limitation.

It should be appreciated that any suitable type, number and organization of process procedures or steps (i.e., agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes, well known to those of ordinary skill in the art), process parameters (i.e., temperature, pressure, pH, process times) or the like can be utilized.

These processes allow a great variety of administration forms. Thus any administration forms and combinations thereof, are contemplated by the present invention. Examples of administration forms include, without limitation, chewable tablet, quick dissolve tablet, effervescent tablet, reconstitutable powder, elixir, liquid, solution, suspension, emulsion, tablet, multi-layer tablet, bi-layer tablet, capsule, soft gelatin capsule, hard gelatin capsule, caplet, lozenge, chewable lozenge, bead, powder, granule, dispersible granule, cachet, douche, suppository, cream, topical, inhalant, aerosol inhalant, patch, particle inhalant, implant, depot implant, dragee, ampoule, ingestible, injectable, infusion, health bar, liquid, food, nutritive food, functional food, yogurt, gelatin, cereal, cereal coating, animal feed or combinations thereof. The preparation of any of the above administrations may be performed by techniques and methods well known and readily available to persons of ordinary skill in the art.

It can be seen, therefore, that the present invention provides a great deal of flexibility in the production of biologically active agent containing sites and biologically compatible arrays as combinations of these sites organized on a common biologically compatible carrier. The quantity of biologically active agent contained in any site can be varied within wide ranges. In addition, various methods of attachment of the system to the biologically compatible carrier are available in order to provide a wide range of flexibility.

The biologically compatible array is then transported by such suitable transport materials disclosed above, or an equivalent effective means, into the station, or stations, wherein the biologically compatible array is secured within the tamper resistant pack.

In an alternate embodiment, the biologically compatible carrier may be placed into one or more components of the tamper resistant pack prior to emplacement of the sites, where such is desired, for example, for improved moisture resistance, without limitation.

It is noted that in other embodiments, preferred user instructions according to the present invention can be applied to the tamper resistant pack by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

In other embodiments, preferred tamper resistant packs can be produced by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

The lids, tabs, safety seals, and bases of the present tamper resistant packs may be embossed, cast deep drawn or vacuum formed out of plastic, plastic laminates, plastic or paper laminates or plastic/metal foil laminates, such as for example, films and film laminates containing PVC, polyamides, polyolefins, polyesters, polycarbonates, a barrier layer against gases and vapors, such barrier layers as metal foils, and also single layer materials, laminates, and multi-layered materials, further comprising one or more layers selected from plastics such as PVC, polyamides, polyolefins, polyesters, and ceramic layers or metallic layers, such as aluminum foil, other like material, variations thereof, and various combinations thereof, without limitation.

Ceramic layers may be produced by chemical vapor deposition and physical vapor deposition or sputtering, which comprise evaporating metals, oxides or nitrides of aluminum, silicon, other metals, organometals and pseudometals in vacuum, and depositing the same on a plastic substrate. These ceramic layers may contain aluminum oxides, silicon oxides, or mixtures of various oxides, or mixtures with metals, such as silicon or aluminum, other like material, variations thereof, and various combinations thereof, without limitation. The single layer materials, laminates, and multi-layered materials of the present invention may comprise metal layers created by evaporating metals in vacuum and depositing the metals on a plastic substrate, such as aluminum layers, for example and without limitation.

The bases usually comprise between 2 and 31 sites, without limitation, and may be surrounded by raised elements forming cavities. The bases are prepared, for example, as an endless strip with the administrations in sites, aligned with the sites by the biologically compatibility carrier, and brought together with the lid material lid form, in the form of an endless strip. The lid then covers the base cavities completely by sealing or adhesive bonding, for example, without limitation. The lid may be sealed or adhesively bonded to the cavities, entirely or partially using suitable tools. The endless strip may then be cut to the desired size, using a stamping tool, for example, without limitation. The cavities may in other embodiments have outer contours, or weakened regions in the lid, safety seal or the base, allowing the tamper resistant pack to be bent or to create breakaway lid elements, making easy removal of the lid segment and separation of the administration possible.

In one embodiment, the tamper resistant pack is configured in a planar formation such that the sites form a series of rows and columns.

In an alternative embodiment, the tamper resistant pack is configured in a rolled formation such that as a strip of sites is pulled, the administration to be taken next is revealed from the container.

In other alternative embodiments, the tamper resistant pack of the present invention can be configured in a sheet formation, rolled formation, multidimensional formation, or any other formation, or combination of formations, without limitation.

Thus, the system of the present invention is not strictly limited to geometries that are planar. Any efficacious geometry is suitable, provided the tamper resistant pack may be produced using biologically compatible components. Non-limiting exemplary geometries include bottles, canisters, packets, tubes, vials, and the like.

It should be appreciated that any suitable type, number and organization of process procedures or steps (i.e., mixing; heating, drying, cooling, addition of components, printing, dot dispensing, extruding, molding, milling, spraying, sifting, screening, atomization, static agitation, mechanical agitation, coating), process parameters (i.e., temperature, pressure, pH, process times) or the like can be utilized.

The tamper resistant pack containing the biologically compatible array is then transported by such suitable transport materials disclosed above, or an equivalent effective means, into the station, or stations, wherein the tamper resistant pack is secured within the outer packaging.

It is noted that in other embodiments, preferred user instructions according to the present invention can be applied to the outer packaging by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

It is further noted that in other embodiments, preferred outer packaging can be produced by at least one method selected from the group consisting of mixing, heating, drying, cooling, addition of components, printing, dot dispensing, extruding, molding, milling, spraying, sifting, screening, atomization, static agitation, mechanical agitation, coating, or like processes, well known to those of ordinary skill in the art.

It should be appreciated that any suitable type, number and organization of process procedures or steps (i.e., mixing, heating, drying, cooling, addition of components, printing, dot dispensing, extruding, molding, milling, spraying, sifting, screening, atomization, static agitation, mechanical agitation, coating), process parameters (i.e., temperature, pressure, pH, process times) or the like can be utilized.

### SUMMARY

In summary, the present invention provides a system, application, and method for delivering sequenced administrations of biologically active agents using a common biologically compatible carrier, enabling compliance with a meticulous administration plan, consisting of varying administrations of biologically active agents over a period of time.

It can be seen that the system, application, and method of the present invention provide several specific new and unexpected benefits, detailed in the advantages listed hereinafter.

It is an advantage of the present invention to provide a system, application, and method of delivering a biologically active agent to an individual utilizing a sequenced biologically compatible delivery system having components which are biologically compatible with the target bodily cavity or area.

Another advantage of the present invention is to provide the system, application, and method which comprises components that have widespread availability.

Another advantage of the present invention is to provide the system, application, and method which are simple.

Another advantage of the present invention is to provide the system, application, and method which are effective.

Another advantage of the present invention is to provide the system, application, and method which are convenient.

Another advantage of the present invention is to provide the system, application, and method which are cost-friendly.

Another advantage of the present invention is to provide the system, application, and method having a plurality of sites of varying isolated formulations of biologically active agents organized on a common biologically compatible carrier.

Another advantage of the present invention is to provide the system, application, and method capable of delivering a wide variety of biologically active agents organized on a common biologically compatible carrier.

Another advantage of the present invention is to provide the system, application, and method capable of delivering large quantities of biologically active agents per administration of a single unit, using only one layer of reagent formulation emplaced on the carrier.

Another advantage of the present invention is to provide the system, application, and method capable of delivering large quantities of biologically active agents per administration of a single unit, using one or more layers of reagent formulation emplaced on the carrier.

Another advantage of the present invention is to provide the system, application, and method capable of delivering user specific types of biologically active agents.

Another advantage of the present invention is to provide the system, application, and method capable of delivering user specific quantities of biologically active agents.

Another advantage of the present invention is to provide the system, application, and method that do not require the user to swallow any form of solid, semi-solid object, or drink any liquid for certain administrations.

Another advantage of the present invention is to provide the system, application, and method capable of improving the palatability of or masking the taste of unpalatable biologically active agents.

Another advantage of the present invention is to provide the system, application, and method that increase the chemical stability of the biologically active agents.

Another advantage of the present invention is to provide the system, application, and method capable of improving the absorption and/or bioavailability of biologically active agents.

Another advantage of the present invention is to provide the system, application, and method capable of improving levels of user errors.

Another advantage of the present invention is to provide the system, application, and method capable of improving user convenience.

Another advantage of the present invention is to provide the system, application, and method capable of improved user monitoring.

Another advantage of the present invention is to provide the system, application, and method capable of improved production variation.

Another advantage of the present invention is to provide the system, application, and method capable of improved distribution.

Another advantage of the present invention is to provide the system, application, and method capable of isolating the biologically active agents in separate sites such that incompatible biologically active agents do not react with other incompatible biologically active agents and thus form unwanted by-products or breakdown the desired biologically active agents.

Another advantage of the present invention is to provide the system, application, and method capable of isolating the biologically active agents within the sites such that incompatible biologically active agents do not react with other incompatible biologically active agents and thus form unwanted by-products or breakdown the desired biologically active agents.

Another advantage of the present invention is to provide the system, application, and method that dispenses precise administrations that are quickly and easily administered by the user.

Another advantage of the present invention is to provide the system, application, and method that allows users to tell how many administrations have been used and how many are left.

Another advantage of the present invention is to provide the system, application, and method that dispenses different ratios and/or combinations and/or concentrations of biologically active agents to be delivered in each administration.

Another advantage of the present invention is to provide the system, application, and method that prevents contamination of the biologically active agents.

Another advantage of the present invention is to provide the system, application, and method that dispenses combinations of various biologically active agents intended for multiple target bodily cavities or areas, in one easy to use system.

Another advantage of the present invention is to provide the system, application, and method that is less bulky than a multitude of jars or tubes and thus can easily be carried conveniently.

Another advantage of the present invention is to provide the system, application, and method that exhibits controlled dissolution, flexibility, non-hygroscopity, palatable flavor and ease of manufacture.

Another advantage of the present invention is to provide the system, application, and method comprising biologically compatible control codes to indicate which biologically active agents are to be emplaced on the biologically compatible carrier.

Another advantage of the present invention is to provide the system, application, and method comprising biologically compatible control codes to indicate where the biologically active agents are to be emplaced on the biologically compatible carrier.

Another advantage of the present invention is to provide the system, application, and method comprising biologically compatible control codes as part of a reliable and effective production control system in that the user can prevent production errors and the user can effectively emplace the proper biologically active agents at each site on the biologically compatible array by comparing the biologically compatible control codes at each site with a master record, through the use of suitably machine readable control codes and suitable machine readers, thus assuring that the correct biologically active agents are emplaced at each site on the biologically compatible array.

Another advantage of the present invention is to provide the system, application, and method comprising biologically compatible control codes that can effectively monitor and verify whether a user has administered the correct biologically active agents.

Another advantage of the present invention is to provide the system, application, and method comprising biologically compatible control codes that can effectively monitor and verify whether a user has administered the correct biologically active agents at the correct time.

Another advantage of the present invention is to provide a sequenced biologically compatible delivery system, application, and method comprising a reliable and effective feedback system in that the user can monitor and determine if the proper administrations have been taken on the proper days and at the proper times by comparing the biologically compatible control codes with a master record, and the user can to effectively monitor and determine if an administration has been missed, prevent improper administrations, or prevent administrations at improper times.

Another advantage of the present invention is to provide the system, application, and method comprising biologically active agents having relatively low melting points that are subject to degradation upon melting.

Another advantage of the present invention is to provide the system, application, and method comprising biologically active agents that are volatile.

Another advantage of the present invention is to provide the system, application, and method that masks disagreeable flavor characteristics.

Another advantage of the present invention is to provide the system, application, and method comprising insoluble components.

Another advantage of the present invention is to provide the system, application, and method comprising chemically incompatible components.

Another advantage of the present invention is to provide the system, application, and method comprising buffer forming reagent formulations which optimize the ratio of ionized and nonionized biologically active agent form.

Another advantage of the present invention is to provide the system, application, and method comprising chemical agents that modify the dissolution characteristics of the biologically active agent.

Another advantage of the present invention is to provide the system, application, and method comprising permeation enhancers that increase absorption of the biologically active agent.

Another advantage of the present invention is to provide the system, application, and method comprising release agents that control the dissolution rate of the administration.

Another advantage of the present invention is to provide the system, application, and method comprising lipid soluble mixtures that increase absorption of the biologically active agent.

Another advantage of the present invention is to provide the system, application, and method comprising dissolution characteristics that can be modified mechanically by changing the compressive forces used to form the reagent formulation.

Another advantage of the present invention is to provide the system, application, and method comprising stratification of biologically active agents.

Additionally, another advantage of the present invention is the system, application, and method comprising both lipophilic and nonlipophilic biologically active agents.

It should be appreciated by those skilled in the art that the present invention provides a system, application, and method for a convenient, cost-friendly, simple and effective way of facilitating precise sequential administration of different formulations of biologically active agents from a common biologically compatible carrier, which can be easily administered by the user. Further, the present invention provides for the user to quickly determine how many administrations have been used, and how many are left. Further, the present invention provides a robust feedback mechanism in the event of user misuse or user error, whereas the user can reliably and effectively monitor compliance with a meticulous administration plan using feedback that compares usage activity with a master record. Further, the present invention provides a positive means to prevent user misuse or user error. Further, the present invention provides a means for the meticulous administration plan to be tailored to the desired specific application and the desired specific user, with the biologically active agents, administration concentration, and volume varying in each site. Further, the present invention provides for each administration to be organized on a common biologically compatible array that prevents errors in production, distribution, and application. These features are particularly critical when present during a meticulous administration plan.

Thus, the present invention accomplishes the objects set forth above.

### EXAMPLES OF THE PRESENT INVENTION

The following examples are given to illustrate various embodiments which have been made or may be made in accordance with the present invention. These examples are given by way of example only, and it is to be understood that the following examples are not comprehensive or exhaustive of the many types of embodiments which can be prepared in accordance with the present invention.

A wide range of changes and modifications to the embodiments of the present invention described herein will be apparent to those skilled in the art and to those who make or use the present invention. The following examples are merely included to illustrate various embodiments only, and are not to be considered as intended to limit the scope of the present invention, which is defined by the following claims as interpreted according to the principles of patent law, including the doctrine of equivalents.

Further, certain examples of the composition of the system given are described with reference to the menstrual cycle application for a female user, without limitation. It should be appreciated by those skilled in the art that many other applications are also possible within the scope of the present invention.

In addition, reference will be made to the use of hormones, without limitation, as the biologically active agent contained within the sites. It should be appreciated by those skilled in the art that many other biologically active agents can be incorporated within the scope of the present invention.

Exemplary embodiments of the invention are provided in the following examples. The following examples of the present invention are presented to illustrate the embodiments produced according to the specifications discussed above in (V) Methods for producing the system of the present invention, to assist one of ordinary skill in making and using the present invention.

### Example 1

9 g of hydoxypropylmethylcellulose (Methocel E15), 8 g of cornstarch, 2 g of lemon extract, 2 g of maltitol, 1 g of dextrose, 1 g of propylene glycol, 0.5 g of maltodextrin, 0.5 g of sucralose, 0.1 g of glyceryl monolaurate (Aldo), and 120 ml of water were mixed at 185 degree F. until a clear solution is formed. The plasticizer is preferably previously dissolved or dispersed in the solvent and added in solution form, along with the surfactant, emulsifying agent, other additives, and any of the biologically compatible carrier components these agents act upon. This improves the function of these agents and therefor the properties of the biologically compatible carriers of the present invention. The resulting solution was cooled to room temperature and coated onto a suitable transport material, for example non-siliconized, polyethylene-coated kraft paper using conventional coating/drying equipment. Coating gap, transport speed, and processing conditions are further specified in (V) Methods for producing the system of the present invention to achieve a dry carrier thickness between 5 and 70 .mu.m.

### Example 2

9 g of hydoxypropylmethylcellulose (Methocel E15), 8 g of cornstarch, 2 g of vanilla extract, 2 g of maltitol, 1 g of dextrose, 1 g of propylene glycol, 0.5 g of maltodextrin, 0.5 g of sucralose, 0.05 g of glyceryl monolaurate (Aldo), 0.05 g of polydimethylsiloxane (FG-10), and 120 ml of water were processed under the conditions as described in the previous example.

### Example 3

9 g of hydoxypropylmethylcellulose (Methocel El 5), 8 g of cornstarch, 2 g of lemon extract, 2 g of maltitol, 1.5 g of gelatin, 1 g of dextrose, 1 g of propylene glycol, 0.6 g of maltodextrin, 0.5 g of sucralose, 0.1 g of adipic acid, 0.1 g of disodium phospate, 0.1 g of fumaric acid, 0.05 g of glyceryl monolaurate (Aldo), 0.05 g of polydimethylsiloxane (FG-10), 0.03 g of aspartame, 0.03 g of acesulfame potassium, 0.02 g of yellow 6, 0.02 g of yellow 5, 0.01 g of butylated hydroxyanisole (BHA), and 120 ml of water were again processed under the conditions as described in the previous Example 1.

### Example 4

9 g of hydoxypropylmethylcellulose (Methocel El 5), 8 g of modified corn starch (B965), 2 g of lemon extract, 2 g of maltitol, 1.5 g of gelatin, 1 g of dextrose, 1 g of propylene glycol, 0.6 g of maltodextrin, 0.5 g of sucralose, 0.1 g of adipic acid, 0.1 g of disodium phospate, 0.1 g of fumaric acid, 0.05 g of glyceryl monolaurate (Aldo), 0.05 g of polydimethylsiloxane (FG-10), 0.03 g of aspartame, 0.03 g of acesulfame potassium, 0.02 g of yellow 6, 0.02 g of yellow 5, 0.01 g of butylated hydroxyanisole (BHA), and 120 ml of water were again processed under the conditions as described in the previous Example 1.

### Example 5

8 g of modified corn starch (B965), 7 g of hydoxypropylmethylcellulose (Methocel E15), 2 g of lemon extract, 2 g of maltitol, 1.5 g of gelatin, 2 g of dextrose, 1.1 g of maltodextrin, 1 g of propylene glycol, 1 g of sucralose, 0.2 g of glyceryl monolaurate (Aldo), 0.1 g of adipic acid, 0.1 g of disodium phospate, 0.1 g of fumaric acid, 0.05 g of polydimethylsiloxane (FG-10), 0.03 g of aspartame, 0.03 g of acesulfame potassium, 0.02 g of yellow 6, 0.02 g of yellow 5, 0.01 g of butylated hydroxyanisole (BHA), and 120 ml of water were again processed under the conditions as described in the previous Example 1.

This portion of the system, as discussed in examples 1 through 5, is then transported by such suitable transport materials disclosed above, or an equivalent effective means, into the station, or stations, wherein the biologically compatible control codes and biologically compatible user instructions are applied.

### Example 6

The black ink jet ink portion of the system, for biologically compatible control codes and biologically compatible user instructions, was prepared by admixing and processing the following ingredients together in a high shear rotor-stator mixer (model HSM-100LC from Ross (Hauppauge, N.Y.)) for five minutes at 1,750 to 2,000 rpm: 62% water, 20% propylene glycol, 10% glycerine, 2% Blue 1, 4% Red 40, and 2% Yellow 6. The speed was then increased to 4,000 to 3,500 rpm. Mixing was continued for 20 to 25 minutes. Then the ink formulation was filtered using a 2.3 .mu.m filter.

The ink was printed onto the biologically compatible carrier to form biologically compatible control codes and biologically compatible user instructions as further specified in (V) Methods for producing the system of the present invention. A high quality print was obtained, the ink quickly dried, and was smear free.

FIG. 1 shows a plan view of a novel sequenced biologically compatible delivery system in accordance with one embodiment of the present invention. The system shown in FIG. 1 generally indicated by arrow 16 contains one row of sites 17. In this embodiment, the system 16 contains four sites containing four administrations 10, 12, 13, and 14. The system 16 is divided into an AM section for morning administrations and a PM section for evening administrations, with the indicia AM and PM serving as biologically compatible user instructions, and suitable barcodes or equivalent indicia serving as biologically compatible control codes. This portion of the system is then transported by such suitable transport materials disclosed above, or an equivalent effective means, into the station, or stations, wherein the sites are applied and secured to the system.

The system is particularly advantageous for hormone treatments, when the administration contained within sites is varied correspondingly with the natural hormone levels during a 28-day or 30-day menstrual cycle. In Examples 7-9 the administration within the site is made up as shown in Tables B-D below:

### Example 7

A suitable mixture for printing was prepared using the following ingredients for a Printing Base Reagent Formulation:

| Ingredient | % |
|---|---|
| butylated hydroxyanisole | .04 |
| glyceryl monolaurate | .8 |
| disodium phosphate | .5 |
| tetrasodium pyrophosphate | .5 |
| citric acid | 1 |
| ribotide | 2 |
| aspartame | 2 |
| wild cherry extract | 3 |
| peppermint extract | 3 |
| vanilla extract | 4 |
| gelatin | 6.16 |
| corn syrup | 25 |
| modified corn starch | 27 |
| water | 25 |
| | 100 |

This portion of the system was further admixed with the biologically active agents.

TABLE B
Example Hormone Administration Plan

| | Week | | | |
|---|---|---|---|---|
| | 1 | 2 | 3 | 4 |
| Component | (%) | (%) | (%) | (%) |
| Printing Base Reagent formulation | 99.99 | 99.83 | 99.83 | 99.99 |
| desogestrel | 0.00 | 0.15 | 0.15 | 0.00 |
| ethinyl estradiol | 0.01 | 0.02 | 0.02 | 0.01 |
| Total | 100 | 100 | 100 | 100 |

The base reagent formulation and biologically active agents were admixed until homogenous. The ingredients were combined in a mixer to ensure uniform distribution of all ingredients within the mixture. The resulting formulations were progressively printed and dried on the biologically compatible carrier, resulting in the preparation of 28 administrations forming a biologically compatible array. The drying temperature and processing conditions are further specified in (V) Methods for producing the system of the present invention, and depends on the length of the drying oven and the transport speed, and has to be adjusted to remove the solvents completely, or almost completely, from this portion of the system.

### Example 8

A suitable mixture for dot dispensing was prepared using the following ingredients for a Dot Dispensing Base Reagent Formulation:

| | |
|---|---|
| butylated hydroxyanisole | .04 |
| glyceryl monolaurate | .8 |
| disodium phosphate | .5 |
| tetrasodium pyrophosphate | .5 |
| citric acid | 1 |
| ribotide | 2 |
| aspartame | 2 |
| wild cherry extract | 3 |
| peppermint extract | 3 |
| vanilla extract | 4 |
| gelatin | 8.16 |
| corn syrup | 26 |
| modified corn starch | 29 |
| water | 20 |
| | 100 |

This portion of the system was further admixed with the biologically active agents.

TABLE C
Example Hormone Administration Plan

| | Week | | | |
|---|---|---|---|---|
| | 1 | 2 | 3 | 4 |
| Component | (%) | (%) | (%) | (%) |
| Dispensing Base Reagent | 99.99 | 99.83 | 99.83 | 99.99 |
| formulation | | | | |
| desogestrel | 0.00 | 0.15 | 0.15 | 0.00 |
| ethinyl estradiol | 0.01 | 0.02 | 0.02 | 0.01 |
| Total | 100 | 100 | 100 | 100 |

The base reagent formulation and biologically active agents were admixed until homogenous. The ingredients were combined in a mixer to ensure uniform distribution of all ingredients within the mixture. Dots totally 28 sites were then dot dispensed onto the biologically compatible carrier and dried, with processing conditions as further specified in (V) Methods for producing the system of the present invention, resulting in the preparation of 28 administrations forming a biologically compatible array.

### Example 9

A suitable mixture for molding was prepared-using the following ingredients for a Molding Base Reagent Formulation

| Ingredient | % |
|---|---|
| citric acid | 1 |
| ribotide | 1 |
| aspartame | 1 |
| compritol 888 | 2 |
| wild cherry microcaps | 3 |
| peppermint microcaps | 3 |
| vanilla microcaps | 4 |
| compressible sugar | 30 |
| maltodextrin | 55 |
| | 100% |

This portion of the system was further admixed with the biologically active agents.

TABLE D
Example Hormone Administration Plan

| | Week | | | |
|---|---|---|---|---|
| | 1 | 2 | 3 | 4 |
| Component | (%) | (%) | (%) | (%) |
| Molding Base Reagent formulation | 99.99 | 99.83 | 99.83 | 99.99 |
| desogestrel | 0.00 | 0.15 | 0.15 | 0.00 |
| ethinyl estradiol | 0.01 | 0.02 | 0.02 | 0.01 |
| Total | 100 | 100 | 100 | 100 |

The ingredients were combined in a mixer to ensure uniform distribution of all ingredients within the mixture. Shots totally 28 sites were then hydraulically compressed onto the biologically compatible carrier, with processing conditions as further specified in (V) Methods for producing the system of the present invention, resulting in the preparation of 28 administrations forming a biologically compatible array.

This portion of the system, as discussed in examples 7 through 9, is then transported by such suitable transport materials disclosed above, or an equivalent effective means, into the station, or stations, wherein the system is secured within the tamper resistant pack, or in such applications where such is not required, directly to the station, or stations, wherein the system is secured within any required packaging and the outer packaging.

### Example 10

One or more systems of the present invention may be housed in a suitable tamper resistant pack. The tamper resistant pack as shown in FIG. 5A, 5B, 6A, and 6B, and further discussed in (II) Tamper resistant pack, assists in compliance with the meticulous administration plans supported by the system.

The completed system, with or without tamper resistant pack is then transported by such suitable transport materials disclosed above, or an equivalent effective means, into the station, or stations, wherein the system is secured within the outer packaging.

### Example 11

One or more systems of the present invention may be stored and secured within any required packaging and the outer packaging. The outer packaging as shown in FIG. 10, and further discussed in (III) Outer packaging, along with printed material and/or audio/visual aids, assists in compliance with the meticulous administration plans supported by the system.

### Example 12

### In Vivo Evaluation

An exemplary embodiment of an application of the present invention is provided herein. This example is presented to illustrate embodiments to assist one of ordinary skill in making and using the same. In this embodiment, the present invention provides an application to treat a user with a biologically active agent, the application including the steps of providing an administration form of a reagent formulation as described above, including a biologically active agent, and the user completing the administration. In this embodiment the application according to the present invention comprises (a) the user administering varying biologically active agents(s) from the biologically compatible array into, onto, or near the target bodily cavity or area of an animal, (b) allowing the biologically active agents(s) to dissolve within, on, or near the target bodily cavity or area; and (c) releasing the biologically active agents into, onto, or near the target bodily cavity or area.

Administrations were delivered to the target oral bodily cavity of several male individuals suffering from migraine every 6 hours using sites from a composition comprising a biologically compatible array prepared according to Examples 4 and 6, and the molding base reagent formulation of Example 9, using the method of the present invention. The users rated their symptoms during the study. The biologically active agents included the following: pseudoephedrine—30 mg, acetaminophen—500 mg, and caffeine—50 mg.

TABLE E
Example Migraine Administration Plan

| Component | Admin1 | Admin2 | Admin3 |
|---|---|---|---|
| Molding Base Reagent formulation | 500 | 460 | 395 |
| Pseudoephedrine | 50 | 40 | 30 |
| Acetaminophen | 425 | 450 | 500 |
| Caffeine | 25 | 50 | 75 |
| Total | 1000 | 1000 | 1000 |

The users reported reduced migraine symptoms such as headache, nausea, light-adversity, and sound-adversity about one hour after treatment. Most symptoms of migraine were completely resolved shortly after one to two treatments. Severity ratings during the application are recorded at one hour post administration.

TABLE F
Severity of migraine headache during study
Severity on a scale of 1-5, 0 = completely resolved

| | | Severity | Severity |
|---|---|---|---|
| | Severity | After administration | After administration |
| User | Start | 1 (1 hr) | 2 (7 hr) |
| 1 | 5 | 1 | 0 |
| 2 | 4 | 1 | 0 |
| 3 | 5 | 0 | 0 |

The present invention being thus described, it will be apparent that the present invention may be varied in many ways. Such variations are not to be regarded as a departure from the spirit and scope of the present invention, all such modifications are intended to be within the scope of the appended claims, and the above description is considered to be that of the preferred embodiments only.

The present invention claimed and desired to be secured by United States Letters Patent is:

1. A composition providing a sequenced biologically compatible delivery system for the sequential release of varying biologically active agents, wherein the improvement comprises:
(I) a biologically compatible array, further comprising
(i) a biologically compatible carrier,
(ii) biologically compatible control codes,
(iii) biologically compatible user instructions,
(iv) a plurality of sequenced sites comprising varying isolated formulations of biologically active agents, and
(v) reagent formulations supporting effective delivery of and admixed with the biologically active agents, and
(II) a tamper resistant pack, and
(III) an outer packaging.

2. The composition of claim 1, wherein the biologically compatible control codes are secured to any portion of the biologically compatible carrier.

3. The composition of claim 1, wherein the biologically compatible control codes define and control the emplacement of the biologically active agents.

4. The composition of claim 1, wherein the biologically compatible control codes are machine readable, by barcode scanners or the equivalent.

5. The composition of claim 1, wherein the biologically compatible control codes correspond to each of the isolated formulations of biologically active agents to increase user compliance with the meticulous administration plan by identifying the isolated formulations of biologically active agents and indicating on which day and at which time the isolated formulations of biologically active agents are to be administered.

6. The composition of claim 1, wherein the biologically compatible user instructions are secured to any portion of the biologically compatible carrier.

7. The composition of claim 1, wherein the biologically compatible user instructions correspond to each of the sites of isolated formulations of biologically active agents to increase user compliance with the meticulous administration plan by identifying the isolated formulations of biologically active agents and indicating on which day the isolated formulations of biologically active agents are to be administered.

8. The composition of claim 1, wherein the biologically active agent comprises at least one biologically active agent selected from the group consisting of biologically acceptable salts, isomers, esters, solvates, derivatives thereof, genetically engineered derivatives thereof, hydrates thereof, hydrophobic compounds, hydrophilic compounds, olefinic compounds, non-olefinic compounds, pH sensitive compounds, non-pH sensitive compounds, anhydrous compounds, and non-anhydrous environment compounds, precursors thereof, mixtures thereof, other like material, and various combinations thereof, without limitation.

9. The composition of claim 1, wherein the biologically active agent of claim 8 is secured to any portion of the biologically compatible carrier to form a site.

10. The composition of claim 9, wherein the site comprises a chewable tablet, quick dissolve tablet, effervescent tablet, reconstitutable powder, elixir, liquid, solution, suspension, emulsion, tablet, multi-layer tablet, bi-layer tablet, capsule, soft gelatin capsule, hard gelatin capsule, caplet, lozenge, chewable lozenge, bead, powder, granules, dispersible granules, cachets, douche, suppository, cream, topical, inhalant, aerosol inhalant, patch, particle inhalant, implant, depot implant, dragee, ampoule, ingestible, injectable, infusion, health bar, liquid, food, nutritive food, functional food, yogurt, gelatin, cereal, cereal coating, or animal feed.

11. The composition of claim 10, wherein the site is formulated for controlled release, namely immediate release, rapid release, pulsatile release, extended release, delayed release, targeted release, targeted delayed release, or mixtures thereof.

12. The composition of claim 10, wherein the site is formulated for oral, buccal, sublingual, nasal, ocular, urethral, buccal, transmucosal, vaginal, inhalational, parenteral, injectable, topical, transdermal, or rectal delivery.

13. An application of the system of claim 1, wherein the user sequentially administers varying biologically active agents to an animal user.

14. The application of claim 13, further characterized by consisting essentially of:
(a) the user administering varying biologically active agents(s) from the biologically compatible array into, onto, or near the target bodily cavity or area of an animal,
(b) allowing the biologically active agents(s) to dissolve within, on, or near the target bodily cavity or area; and
(c) releasing the biologically active agents into, onto, or near the target bodily cavity or area.

15. The application of claim 13, wherein the biologically active agent exhibits absorption problems due to solubility limitations.

16. The application of claim 13, wherein the biologically active agent is subject to degradation in the gastrointestinal tract.

17. The application of claim 13, wherein the biologically active agent is subjected to extensive metabolism.

18. A method for producing the system of claim 1, comprising:
producing the biologically compatible carrier using at least one process selected from the group comprising agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes;
emplacement of the biologically compatible control codes on the biologically compatible carrier using at least one process selected from the group comprising agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes;
emplacement of the biologically compatible user instructions on the biologically compatible carrier using at least one process selected from the group comprising agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes;
emplacement of a plurality of sites of varying isolated formulations of biologically active agents, admixed with reagent formulations on the biologically compatible carrier, using at least one process selected from the group comprising agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, mixing, heating, drying, cooling, addition of components, printing, dot dispensing, extruding, molding, milling, spraying, sifting, screening, atomization, static agitation, mechanical agitation, coating, slurry molding, dry molding, wet molding, hot molding, cold molding, slugging, dehydration, freeze drying (lyophilization), spraying, vapor deposition, or like processes;
securing the biologically compatible arrays in tamper resistant packs, and placing and storing the tamper resistant packs in outer packaging.
